(19) United States
(12) Patent Application Publication
(10) Pub. No.: US 2026/0035696 A1
(43) Pub. Date: Feb. 5, 2026

(54) COMBINATION TREATMENT FOR CANCER

(71) Applicant: THE UNIVERSITY OF CHICAGO, Chicago, IL (US)

(72) Inventors: Ralph R. WEICHSELBAUM, Chicago, IL (US); Chuan HE, Chicago, IL (US); Liangliang WANG, Chicago, IL (US); Linda ZHANG, Chicago, IL (US); Hua L. LIANG, Chicago, IL (US); Sean PITRODA, Chicago, IL (US); Andras PIFFKO, Chicago, IL (US); Steven CHMURA, Chicago, IL (US)

(73) Assignee: THE UNIVERSITY OF CHICAGO, Chicago, IL (US)

(21) Appl. No.: 19/103,261

(22) PCT Filed: Aug. 17, 2023
(86) PCT No.: PCT/US2023/072359

§ 371 Date: Feb. 12, 2025

Related U.S. Application Data
(60) Provisional application No. 63/398,665, filed on Aug. 17, 2022.

(51) Int. Cl. C12N 15/113; A61K 9/00; A61K 39/00; A61K 45/06; A61P 35/00; C07K 16/28; C12Q 1/6886; G01N 33/68

(52) U.S. Cl. CPC C12N 15/113; A61K 9/0019; A61K 45/06; A61P 35/00; C07K 16/2818; C12Q 1/6886; G01N 33/6893; A61K 2039/505; C12N 2310/14; C12N 2320/31; C12Q 2600/106; G01N 2333/4703; G01N 2800/52

(57) ABSTRACT

Aspects of the present disclosure are directed to methods and compositions for diagnosing and/or treating a subject having cancer. Certain aspects relate to treatment with a cancer therapy and a YTHDF2 inhibitor or a pharmaceutical composition comprising a cancer therapy and a YTHDF2 inhibitor. In some aspects, the YTHDF2 inhibitor includes an oligonucleotide targeting YTHDF2 mRNA or a small molecule inhibitor of YTHDF2. In some aspects, a subject has been determined to have or to have had resistance to a previous cancer treatment, such as radiotherapy and/or immunotherapy.

### CROSS-REFERENCE TO RELATED APPLICATIONS

This application claims the benefit of priority to U.S. Provisional Patent Application Ser. No. 63/398,665 filed Aug. 17, 2023, which is hereby incorporated by reference in its entirety.

### STATEMENT REGARDING FEDERALLY SPONSORED RESEARCH OR DEVELOPMENT

This invention was made with government support under grant number HG008935, and CA262508 awarded by the National Institutes of Health. The government has certain rights in the invention.

### BACKGROUND

### I. Technical Field

Aspects of this disclosure relate to at least the fields of cancer biology, radiology, immunology, and medicine.

### II. Background

Radiotherapy and immunotherapy are known to be effective for treatment of cancer. However, radiotherapy can have adverse immunogenic and immune suppressive effects, which may be due to infiltration of myeloid-derived suppressor cells (MDSCs) that express or secrete immunoregulatory factors (Engblom et al., 2016) to downregulate cytotoxic CD8+ T cell function. Thus, there remains a need for methods and compositions for inhibiting the immunosuppressive effects of radiotherapy and immunotherapy to sensitize cancers to these cancer therapies.

Radiation therapy (RT) is employed in 50%-60% of cancer patients.^{1,2} Despite continuous technological and therapeutic improvements, the majority of patients experience treatment failure either locally (due to tumor radioresistance) or at distant metastatic sites. Tumor cell radioresistance contributes to treatment failure, and radiosensitizing therapeutic strategies have generally focused on inhibiting DNA repair or increasing DNA damage.³ The immune contexture is vital in radiocurability.⁴ Pre-clinical data indicate an immune stimulatory effect of ionizing radiation (IR) alone or in combination with checkpoint blockade.⁵ Encouraged by these promising preclinical results, an increasing number of clinical trials combining checkpoint blockade with IR have been launched (reviewed in Pointer et al. and Turchan et al.^{6,11}). Despite encouraging individual patient responses, only two randomized trials have shown positive results in terms of improving survival.^{12,13} These trials utilized checkpoint blockade after chemo/radiotherapy to inhibit microscopic metastatic disease; however a consistent meaningful interaction between checkpoint immunotherapy and radiotherapy in humans has yet to be established.¹⁴

Myeloid-derived suppressor cells (MDSCs) have emerged as crucial negative regulators of the antitumor immune response. Inhibiting immunosuppressive effects of cancer therapy is a pivotal step toward therapeutic success.¹⁵ In the setting of cancer, MDSCs maintain cellular plasticity and can be reprogrammed into various myeloid cells depending on the tumor and normal tissue microenvironments as well as various treatments.^{16,17} Mechanisms of immune suppression by MDSCs include secretion or expression of immunoregulatory factors,^{18,19} which downregulate cytotoxic CD8+ T cell function. Tumor infiltration of MDSCs may account for preclinical/clinical radio- and immune checkpoint blockade resistance.¹⁹⁻²¹ In preclinical studies, both fractionated and hypo-fractionated treatment regimens resulted in MDSC expansion,²²⁻²⁵ which led to tumor radioresistance.^{24,26,27} Clinical studies also showed that radiotherapy induces MDSCs expansion^{24,25,28,29} and immunosuppression,³⁰ and these effects appeared to associate with adverse patient outcomes.^{25,31-33} There are several ongoing or recently completed phase 1 and 2 clinical trials aiming to modulate MDSCs to enhance immunotherapy in distinct ways.^{19,34} Examples include MDSC depletion,³⁵ MDSC migration blockade (20 trials using CCR5/CCR2 antagonists and 52 trials using CSFR antagonists),³⁶ MDSC function blockage to attenuate their suppressive effects (targeting phosphodiesterase 5 to reduce iNOS and ARG1 production),³⁷ and induction of MDSC differentiation by utilizing all-trans-retinoic acid.³⁸ Despite these studies, no randomized trials have shown improved survival.

N⁶-methyladenosine (m⁶A), the most prevalent eukaryotic mRNA modification, regulates the stability and translation of modified mRNAs.^{39,40} m⁶A is dynamically regulated by “writers” (methyltransferase complex: METTL3, METTL14, and WTAP) that install m⁶A methylation, “erasers” (demethylases: FTO and ALKBH5) that remove m⁶A marks, and “readers” (YTHDF1/2/3 and YTHDC1/2) that recognize m⁶A-modified RNA to regulate RNA metabolisms.⁴¹ Among the m⁶A reader proteins, YTHDF1 facilitates mRNA translation; YTHDF2 promotes mRNA degradation; and YTHDF3 promotes both translation and RNA degradation depending on the biological context.⁴²⁻⁴⁴ Recent studies have suggested m⁶A readers and erasers are implicated in tumor growth in various cancer types.⁴⁵⁻⁴⁹ Only a few studies have reached the impact of reader proteins on antitumor immune response: loss of Ythdf1 enhanced the cross-priming activity via decreasing lysosomal proteases in classical dendritic cells and suppressed tumor growth.⁵⁰ YTHDF2 affects many biological processes including cell cycle progression,⁵¹ response to stress,⁵² and regulation of hematopoietic stem cell expansion.^{47,53-55} However, the intrinsic role of YTHDF2 in immune cells especially in relation to radiotherapy and immunotherapy has not been explored in depth.

### SUMMARY

Aspects of the present disclosure address needs in the art by providing methods and compositions for treating subjects with cancer (e.g., lung cancer) and for inhibiting the immunosuppressive effects of radiotherapy and/or immunotherapy to sensitize subjects having cancers to these therapies. Accordingly, provided herein, in some aspects, are methods and compositions for treating a subject with cancer comprising administering to the subject a therapeutically effective amount of a YTHDF2 inhibitor and a cancer therapy. In some aspects, the YTHDF2 inhibitor comprises a small molecule compound or a pharmaceutical composition comprising a small molecule compound. In some aspects, the subject has been diagnosed with or is suspected of having cancer. In some aspects, the disclosed methods comprise providing the YTHDF2 inhibitor and the cancer therapy to a subject who was previously treated for cancer and who was determined to be resistant to the previous treatment. In some aspects, the previous cancer treatment comprised a radiotherapy and/or an immunotherapy. In some aspects, administration of the YTHDF2 inhibitor decreases suppression of an immune response to the cancer therapy. In some aspects, administration of the YTHDF2 inhibitor inhibits NF-κB signaling in myeloid-derived suppressor cells in the subject. In some aspects, administration of the YTHDF2 inhibitor decreases myeloid-derived suppressor cell trafficking and function in the subject.

Aspects of the disclosure include methods and compositions for treating a subject having cancer, methods and compositions for increasing the efficacy of a cancer therapy in a subject, methods for diagnosing a subject with cancer, methods for prognosing a subject with cancer, methods and compositions for sensitizing a subject with cancer to radiotherapy or immunotherapy, methods and compositions for inhibiting the immunosuppressive effects of radiotherapy and/or immunotherapy to sensitize subjects having cancers to radiotherapy and/or immunotherapy, methods and compositions for inhibiting NF-κB signaling in a subject, methods and compositions for decreasing myeloid-derived suppressor cell trafficking and function in a subject, methods for identifying a subject with cancer as a candidate for a combination therapy, methods and compositions for treating a subject having lung cancer, compositions comprising a YTHDF2 inhibitor and a cancer therapy, compositions comprising an oligonucleotide targeting YTHDF2 mRNA, and compositions comprising small molecule inhibitors of YTHDF2

Methods of the disclosure can include 1, 2, 3, 4, 5, 6, 7, 8, 9, 10, 11, 12, or more of the following steps: providing a radiotherapy to a subject; providing an immunotherapy to a subject; providing a YTHDF2 inhibitor to a subject; providing a YTHDF2 inhibitor and a cancer therapy to a subject; providing a YTHDF2 inhibitor, a cancer therapy, and one or more additional cancer therapies to a subject; determining a subject to have cancer; providing two or more types of cancer therapy to a subject; identifying a subject as having had resistance to a previous cancer treatment; testing a subject for resistance to previous cancer treatments; identifying a subject as being a candidate for a combination therapy comprising a YTHDF2 inhibitor and a cancer therapy; measuring a level of a gene product, which in certain aspects is a YTHDF2 gene product; measuring a level of a YTHDF2 gene product in peripheral blood mononuclear cells; administering an additional therapy based on a measure level of a YTHDF2 gene product; and comparing a level of a gene product, which in certain aspects is a YTHDF2 gene product, to a standard. Certain aspects of the disclosure may exclude one or more of the preceding elements and/or steps.

Disclosed herein, in some aspects, is a method for treating cancer in a subject, the method comprising administering to the subject a therapeutically effective amount of (a) a YTHDF2 inhibitor; and (b) a cancer therapy. Also disclosed herein, in some aspects, is a method of treating cancer in a subject, the method comprising inhibiting NF-κB signaling in the subject by administering to the subject a therapeutically effective amount of (a) a YTHDF2 inhibitor; and (b) a cancer therapy. Further disclosed herein, in some aspects, is a method of treating cancer in a subject, the method comprising decreasing myeloid-derived suppressor cell trafficking and function in the subject by administering to the subject a therapeutically effective amount of (a) a YTHDF2 inhibitor; and (b) a cancer therapy. Also disclosed herein, in some aspects, is a method of increasing the efficacy of a cancer therapy in a subject, the method comprising administering to the subject a therapeutically effective amount of (a) a YTHDF2 inhibitor; and (b) the cancer therapy.

Additionally disclosed herein, in some aspects, is a method of inhibiting NF-κB signaling in myeloid-derived suppressor cells in a subject, the method comprising administering to the subject a therapeutically effective amount of a YTHDF2 inhibitor. Further disclosed is a method of decreasing myeloid-derived suppressor cell trafficking and function in a subject, the method comprising administering to the subject a therapeutically effective amount of a YTHDF2 inhibitor. In some aspects, the method further comprises administering to the subject a cancer therapy. The amount administered to the subject can comprise a therapeutically effective amount of the cancer therapy, which can be determined by a skilled practitioner.

Also disclosed herein, in some aspects, is a pharmaceutical composition comprising (a) a YTHDF2 inhibitor and (b) a cancer therapeutic. In some aspects, the cancer therapeutic is an immunotherapeutic. In some aspects, the immunotherapeutic is an immune checkpoint inhibitor. in some aspects, the pharmaceutical composition further comprises a pharmaceutically acceptable carrier. The pharmaceutical composition can be formulated for any suitable administration. In certain aspects, the pharmaceutical composition is formulated for injection, oral administration, intraperitoneal administration, subcutaneous administration, topical administration, intradermal administration, inhalation, intrapulmonary administration, rectal administration, vaginal administration, sublingual administration, intramuscular administration, intravenous administration, intraarterial administration, intrathecal administration, or intralymphatic administration.

In some aspects, the cancer therapy comprises immunotherapy. In some aspects, the immunotherapy comprises checkpoint blockade therapy. In some aspects, the cancer therapy comprises radiotherapy. In some aspects, the YTHDF2 inhibitor is an oligonucleotide targeting YTHDF2 mRNA. In some aspects, the oligonucleotide is a YTHDF2-targeting siRNA, shRNA, or antisense oligonucleotide. In some aspects, the oligonucleotide is an inhibitor of interaction of YTHDF2 protein with m⁶A-containing mRNA. In some aspects, the YTHDF2 inhibitor is a small molecule inhibitor of YTHDF2. In some aspects, the YTHDF2 inhibitor and the cancer therapy are administered substantially simultaneously. In some aspects, the YTHDF2 inhibitor and the cancer therapy are administered sequentially. In some aspects, the YTHDF2 inhibitor is administered before the cancer therapy. In some aspects, the YTHDF2 inhibitor is administered after the cancer therapy. In some aspects, the YTHDF2 inhibitor and the cancer therapy are administered via the same route of administration. In some aspects, the YTHDF2 inhibitor and the cancer therapy are administered via different routes of administration.

In some aspects, the method further comprises administering to the subject an additional cancer therapy. In some aspects, the additional cancer therapy comprises immunotherapy. In some aspects, the immunotherapy comprises checkpoint blockade therapy. In some aspects, the additional cancer therapy comprises radiotherapy. In certain aspects, the cancer is a solid tumor. In certain aspects, the cancer is a blood cancer. In certain aspects, the cancer is metastatic. In some aspects, the cancer is glioma, sarcoma, liver, lung, colon, or melanoma.

In some aspects, the subject was previously treated for the cancer. In some aspects, the subject was determined to be resistant to the previous treatment. In some aspects, the previous treatment comprised radiotherapy or immunotherapy. In certain aspects, the subject has not been previously treated for cancer.

In some aspects, administration of the YTHDF2 inhibitor decreases suppression of an immune response to the cancer therapy. In some aspects, administration of the YTHDF2 inhibitor decreases myeloid-derived suppressor cell trafficking and function in the subject. In some aspects, administration of the YTHDF2 inhibitor decreases myeloid-derived suppressor cell trafficking and function in the subject. In certain aspects, the subject was determined to have a suppressed immune system. In certain aspects, the subject was determined to be at risk of a suppressed immune system response. The suppressed immune system response may be measured and determined by a skilled practitioner. The suppressed immune system response may comprise a suppression and/or reduction in the amount of immune cells that are activated, including in response to the cancer therapy. The suppressed immune response may comprise a response that activates fewer immune cells than what would be activated in a different individual, including an individual that responded to the cancer therapy. In certain aspects, the suppressed immune system response is relative to a standard. The standard may be determined by a skilled practitioner and may be a response measured in a different individual or population of individuals. The standard may be a measured immune response from an individual that was responsive to the cancer therapy.

Also disclosed are methods of detecting a YTHDF2 gene product in a cancer patient, methods of determining YTHDF2 gene product levels in a patient, methods of detecting a YTHDF2 gene product in peripheral blood mononuclear cells (PBMCs), methods of determining YTHDF2 gene product levels PBMCs, methods of diagnosing a cancer patient based on YTHDF2 gene product levels, methods of prognosing a cancer patient based on YTHDF2 gene product levels, methods of determining a likelihood of a cancer patient's responsiveness to a radiotherapy and/or immunotherapy, methods of determining a likelihood of disease progression (including local and/or distal tumor progression and/or metastasis progression), and methods of treating a cancer patient.

Certain methods comprise measuring a level of YTHDF2 in PBMCs taken from at least one biological sample from a cancer patient. In certain aspects, The biological sample may be purified, modified, and/or processed to enrich for PBMCs. In certain aspects, the PBMCs are isolated by a cell purification method, such as an affinity purification and/or cell sorting method. In certain aspects, the PBMCs comprise myeloid-derived suppressor cells. Certain methods comprise measuring a level of a YTHDF2 gene product in a population myeloid-derived suppressor cells (MDSCs). In certain aspects, the MDSCs are derived, purified, isolated, and/or enriched from PBMCs. In certain aspects, the PBMCs are enriched for MDSCs. In certain aspects, non-myeloid-derived suppressor cells are removed from the PBMCs.

In certain aspects, at least one of the biological samples is taken from the cancer patient after the cancer patient has received radiotherapy. In certain aspects, at least one of the biological samples is taken at least, at most, or approximately 1, 2, 3, 4, 5, 6, 7, 8, 9, 10, 11, 12, 13, 14, 15, 16, 17, 18, 19, 20, 21, 22, 23, 24, or more (or any range derivable therein) minutes, hours, days, weeks, or months after the cancer patient has received radiotherapy. In certain aspects, at least one of the biological samples is taken during a dosing regimen of an immunotherapy provided to the cancer patient. A dosing regimen may comprise one or more administrations of a therapy provided over a set period of time, as determined by a practitioner. A dosing regimen may comprise 1, 2, 3, 4, 5, 6, 7, 8, 9, 10 or more (or any range derivable therein) administrations of the immunotherapy, which may be 1, 2, 3, 4, 5, 6, 7, 8, 9, 10, 11, 12, 13, 14 or more (or any range derivable therein) days or weeks apart. In certain aspects, at least one of the biological samples is taken at least, at most, or approximately 1, 2, 3, 4, 5, 6, 7, 8, 9, 10, 11, 12, 13, 14, 15, 16, 17, 18, 19, 20, 21, 22, 23, 24, or more (or any range derivable therein) minutes, hours, days, weeks, or months after the cancer patient has received an immunotherapy. In certain aspects, the cancer patient has lung cancer. In certain aspects, the cancer patient is suspected of having lung cancer. In certain aspects, the cancer patient is diagnosed with having lung cancer. In certain aspects, the cancer patient has, is suspected of having, or is diagnosed with having glioma, sarcoma, liver cancer, colon cancer, or melanoma.

Certain methods comprise measuring the level of one or more YTHDF2 gene products. In certain aspects, the level of one or more YTHDF2 proteins is measured. In certain aspects, the level of one or more YTHDF2 RNAs, such as a YTHDF2 mRNA, is measured.

In certain aspects, the immunotherapy comprises a checkpoint blockade therapy. The checkpoint blockade therapy may comprise pembrolizumab. The checkpoint blockade therapy may comprise ipilimumab. In certain aspects, the checkpoint blockade therapy is not pembrolizumab. In certain aspects, the checkpoint blockade therapy is not ipilimumab.

In certain aspects, the method comprises comparing the level of a YTHDF2 gene product to a standard. The standard may be a level of a YTHDF2 gene product present in PBMCs taken from a patient that responded to radiotherapy and/or immunotherapy. The standard may be a level determined by a skilled practitioner to be useful in methods described herein. The standard may be a level measured in a healthy individual. The standard may be a level known and/or measured to correlate with disease progression.

In certain aspects, the method comprises administering an additional therapy to the cancer patient based on a measured level of a YTHDF2 gene product. In certain aspects, the additional therapy comprises a cancer therapy, including any cancer therapy disclosed herein. In certain aspects, the additional therapy comprises a YTHDF2 inhibitor, including any YTHDF2 inhibitor disclosed herein. In certain aspects, the additional therapy comprises a radiotherapy and/or immunotherapy. In some aspects, the immunotherapy comprises a checkpoint blockade therapy. In some aspects, the checkpoint blockade therapy comprises pembrolizumab and/or ipilimumab.

Also disclosed are methods of determining a likelihood of a cancer patient's responsiveness to a radiotherapy and/or immunotherapy, the method comprising measuring a level of a YTHDF2 gene product in peripheral blood mononuclear cells (PBMCs) taken from at least one biological sample from the cancer patient. In certain aspects, measuring of a YTHDF2 gene product occurs before an administration of the radiotherapy and/or immunotherapy. In certain aspects, measuring of a YTHDF2 gene product occurs after an administration of the radiotherapy and/or immunotherapy.

In some aspects, the cancer patient has, is suspected of having, or is diagnosed with having lung cancer, glioma, sarcoma, liver cancer, colon cancer, or melanoma.

“Individual, “subject,” and “patient” are used interchangeably and can refer to either a human or non-human, such as primates, mammals, and vertebrates. In particular aspects, the subject is a human. The subject is of any age, gender, or race. The subject can be a patient, e.g., have or be suspected of having a disease (that may be referred to as a disorder or medical condition), such as cancer. The subject may be undergoing or have undergone treatment. The subject may be asymptomatic. The subject may be a healthy individual desirous of prevention of a disease or condition.

Throughout this application, the terms “about,” “substantially.” and “approximately” are used according to their plain and ordinary meaning in the area of cell and molecular biology to indicate a deviation of ±10% of the value(s) to which they are attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one.” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any aspect discussed in this specification can be implemented with respect to any method or composition of the disclosure, and vice versa and that different aspects may be combined. Furthermore, compositions of the disclosure can be used to achieve methods of the disclosure. The claims originally filed are contemplated to cover claims that are multiply dependent on any filed claim or combination of filed claims.

Other objects, features and advantages of the present disclosure will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1F. Local tumor irradiation increases tumor-associated myeloid cells expressing YTHDF2. (FIG. 1A) UMAP plot of scRNA seq data showing the different myeloid cell clusters of CD45⁺ immune cells, which were isolated from non-irradiated (Control) and irradiated (IR) MC38 mouse tumors, respectively (left). Bar plot showing the proportion of ‘P01:Ly6c2_Mono’ cluster in control and irradiated tumors, respectively (right). (FIG. 1B) Expression levels of selected genes identifying P01 cluster as MDSC in UMAP space. (FIG. 1C) Flow cytometry analysis of MDSCs in PBMCs from cancer patients with lung metastasis (pre-RT vs. post-RT). (FIG. 1D) Heatmap showing the mRNA expression of m⁶A-related genes (identified by qPCR analysis) in MDSCs from non-irradiated and irradiated MC38 tumors. (FIG. 1E) Mean Fluorescent Intensity (MFI) of YTHDF2 in myeloid cells of PBMCs from non-responders patients pre- and post-RT by flow cytometry. (FIG. 1F) Representative flow cytometry analysis of YTHDF2 expression in MC38 tumor-infiltrating MDSCs (CD45⁺CD11b⁺Ly6C^{hi}). Data are represented as mean±s.e.m., two-sided unpaired Student's t-test (FIGS. 1C, 1E).

FIGS. 2A-2G. Ythdf2 deficiency in myeloid cells improves response to radiotherapy. (FIGS. 2A-2B) Wild-type (Ythdf2^{fl/fl}) or Ythdf2-cKO (Lyz^{cre}Ythdf2^{fl/fl}) mice were injected subcutaneously with 2×10⁶ MC38 cells. When the tumor size reached 100 mm³, tumor-bearing mice were treated with tumor-local IR (20 Gy, one dose). Tumor growth was monitored (FIG. 2A). Survival was monitored (FIG. 2B). Mice with tumor volumes less than 200 mm³ were considered to be surviving. (FIGS. 2C, 3D) Wild-type or Ythdf2-cKO mice were injected subcutaneously with 10⁶ B16-OVA cells (FIG. 2C) or 10⁶LLC cells (FIG. 2D). When the tumor size reached 100 mm³, tumor-bearing mice were treated with local IR (20 Gy, one dose). Tumor growth was monitored. (FIG. 2E) Lung metastasis in WT or Ythdf2-cKO mice 22 days after flank LLC injection. Treatments as indicated in (FIG. 2D). Size of lung metastases was measured. (FIG. 2F) Populations of MC38 tumor-infiltrating immune cells assessed by flow cytometry (treatment conditions as indicated). (FIG. 2G) The numbers (left) and percentage (right) of tumor-infiltrating CD11b⁺Ly6C^{hi} cells three days after IR, as assessed by flow cytometry. Data are represented as mean±s.e.m., two-sided unpaired Student's t-test (FIGS. 2A, 2C, 2D), two-sided log-rank (Mantel-Cox) test (FIG. 2B) or one-way ANOVA with Bonferroni's multiple comparison tests (FIGS. 2E, 2G). *P<0.05, **P<0.01, and ***P<0.001.

FIGS. 3A-3I. IR and YTHDF2 inhibition reshapes the composition of MDSC populations in blood and tumors. (FIG. 3A) UMAP plot displaying different clusters of mMDSCs from scRNA-seq. The CD45⁺CD11b⁺Ly6C^{hi} cells were isolated from blood and tumors in IR-treated MC38 tumor-bearing mice, respectively. (FIG. 3B) Cell proportion changes (IR vs. non-IR) of different mMDSC cell subsets in blood and tumors, respectively. (FIG. 3C) Cell trajectory of cell populations in blood (only including monocytes and macrophages) were visualized using UMAP. (FIG. 3D) Cell trajectory of cell populations in tumors (only including monocytes and macrophages) were visualized using UMAP. (FIG. 3E) Proportion of different mMDSC cell subsets in tumors with non-IR versus IR treatment. (FIG. 3F) Proportion of mMDSC cell subsets in blood and tumors from WT and Ythdf2-cKO mice with non-IR versus IR treatment (left); Cell proportion changes of mMDSC cell subsets in tumors in WT+IR vs. WT and Ythdf2-cKO+IR vs. WT+IR (right). (FIG. 3G) Cell trajectory of combined cell populations in blood and tumors from WT or Ythdf2-cKO mice. (FIG. 3H) Expression level of gene signatures of C15 in UMAP space. (FIG. 3I) Proportion of C15 and C9 clusters from blood and tumors, respectively (Ythdf2-cKO+IR versus WT+IR).

FIGS. 4A-4F. YTHDF2 controls MDSC migration and suppressive function in the context of IR. (FIG. 4A) MDSCs were sorted from MC38 tumors, as indicated in (FIG. 2A), and subjected to the trans-well migration assay. Migrated cells on the trans-well membranes were visualized under a light microscope and quantified. (FIG. 4B) MC38 tumor fragments from WT or Ythdf2-cKO mice were transplanted into CD45.1 WT mice. Ten days later, tumors were treated with local IR (20 Gy, one dose). Three days after IR, the number of tumor-infiltrating CD45.1+CD11b⁺Ly6C^{hi} cells was determined by flow cytometry. (FIG. 4C) MC38 tumor fragments from CD45.1 WT mice were transplanted into WT or Ythdf2-cKO mice (CD45.2). Ten days later, tumors were treated with local IR (20 Gy, one dose). Three days after IR, the number of tumor-infiltrating CD45.2⁺CD11b⁺Ly6C^{hi} cells was determined by flow cytometry. (FIG. 4D) CD11b⁺ myeloid cells were sorted from MC38 tumors, as indicated in FIG. 2A, and subjected to bulk mRNA-seq. Heatmap of functional enrichment analysis of differentially expressed gene pathways. (FIG. 4E) Violin plot of gene expression fold changes (log 2FC) in genes related to chemokine signaling pathways, cell migration, and positive regulation of cell migration pathways (comparing IR versus Control, and Ythdf2-cKO+IR versus WT+IR). (FIG. 4F) Flow cytometry analysis of an in vitro proliferation assay showing the frequency of proliferating CD8⁺ T cells when co-cultured with MDSCs sorted from different MC38 tumors, as indicated. Data are represented as mean±s.e.m., one-way ANOVA with Bonferroni's multiple comparison tests (FIGS. 4A-4C, 4F). *P<0.05. **P<0.01, ***P<0.001, and ****P<0.0001.

FIGS. 5A-5F. IR-induced YTHDF2 enhances NF-κB signaling by promoting m⁶A-modified RNA degradation. (FIG. 5A) Gene Set Enrichment Analysis (GSEA) of differentially expressed genes following IR treatment (IR vs. Ctrl) against ranked list of genes according to expression changes comparing Ythdf2-cKO+IR versus WT+IR. (FIG. 5B) Venn diagram of overlapping genes that were downregulated following IR vs. Ctrl and upregulated following Ythdf2-cKO+IR vs. WT+IR (top); or upregulated upon IR vs. ctrl and downregulated upon Ythdf2-cKO+IR vs. WT+IR (bottom). (FIG. 5C) Volcano plot of genes with differential expression levels in CD11b⁺ myeloid cells (IR vs. Ctrl). M⁶A marked genes are shown with orange circles. Downregulated genes (downDEGs) are highlighted with blue and upregulated genes (upDEGs) with red. (FIG. 5D) Boxplot showing gene expression log 2FC comparing WT+IR vs. WT+ctrl (left); and Ythdf2-cKO+IR vs. WT+IR (right). Genes were categorized into two groups according to whether they were marked with m⁶A or not (non-m⁶A). (FIG. 5E) Scatter plot of YTHDF2 binding intensity on its target genes (Ctrl vs. IR). (FIG. 5F) Heatmap showing gene expression level in WT mice with non-IR (WT+Ctrl) and IR (WT+IR) treatment, and Ythdf2-cKO mice with IR treatment (cKO+IR) (left). Genes were further categorized into groups according to whether they were bound by YTHDF2, or marked with m⁶A (right).

FIGS. 6A-6D. Pharmacological inhibition of YTHDF2 enhances responses to radiotherapy and immunotherapy. (FIG. 6A) Inhibitory activities (IC50) of Inhibitor A (red) and Inhibitor B (blue) against YTHDF2 binding to m⁶A determined by AlphaScreen assay (right). Data are presented as mean±SD. (FIG. 6B) Wild-type mice were injected subcutaneously with 2×10⁶ MC38 cells. When the tumor size reached 100 mm³, tumors were treated with local IR (20 Gy, one dose). On the same day, the mice were treated with Inhibitor B (9 μg/per mice, daily) until the end of the experiment. Tumor growth was monitored. (FIG. 6C) WT mice were injected subcutaneously with 2×10⁶ MC38 cells. When the tumor size reached 100 mm³, tumors were treated with local IR (20 Gy, one dose), anti-PD-L1 antibody (2 doses per week, three doses total) and/or Inhibitor B (9 μg/per mice, daily), as indicated. Tumor growth was monitored. (FIG. 6D) The numbers of tumor-infiltrating CD11b⁺Ly6C^{hi} cells in mice, with different treatment as indicated, were assessed by flow cytometry.

FIGS. 7A-7D. scRNA-seq identified CD45⁺ immune cell populations and myeloid subsets. (FIGS. 7A-7B) UMAP showing five clusters including T cells, NK, Macrophages, DCs and Monocytes (a) from scRNA-seq using CD45⁺ sorted immune cells from MC38 tumors with and without IR treatment, respectively (b). (FIG. 7C) Proportion of different cell subsets of CD45⁺ immune cells in irradiated vs. non-irradiated MC38 tumors (left). Changes in proportion of different cell subsets of CD45⁺ immune cells in irradiated vs. non-irradiated MC38 tumors (right). (FIG. 7D) Proportion of different cell subsets of myeloid cells from of CD45⁺ immune cells in irradiated vs. non-irradiated MC38 tumors.

FIGS. 8A-8C. IR-induced YTHDF2 in MDSCs. (FIG. 8A) Flow cytometric analysis of YTHDF2 expression in different immune cells (as indicated) isolated from irradiated MC38 tumors. (FIG. 8B) MDSCs sorted from tumors in MC38-bearing WT mice one, two, and three days after IR. Immunoblot analysis of YTHDF2 in sorted MDSCs. (FIG. 8C) MDSCs were derived from bone marrow (WT mice) and treated with different doses of IR and cultured for different times as indicated. Graph showing the immunoblot analysis of YTHDF2. Data are represented as mean±s.e.m., two-sided unpaired Student's t-test (FIG. 8A). *P<0.05 and **P<0.01.

FIGS. 9A-9G. The antitumor effects of IR in Ythdf2-cKO mice depend on CD8+ T cell response. (FIG. 9A) MDSCs (CD11b⁺Ly6C^{hi}) were sorted from spleen in CD45.1 WT mice and used for adoptive transfer into MC38 tumor bearing Ythdf2-cKO mice (CD45.2). On the same day, the mice were treated with local tumor irradiation (20 Gy, one dose). Tumor growth was monitored. (FIG. 9B) The numbers of MC38 tumor-infiltrating total CD8⁺ T cells (left) and CD8⁺IFNγ⁺ T cells (right) in WT or Ythdf2-cKO mice with/without IR (5 days after IR). (FIG. 9C) The CD8⁺ T cells from MC38 tumors (in b) were isolated and the IFN-γ spots were enumerated by ELISPOT assay. (FIG. 9D) The MC38 tumor tissues (in b) were collected to measure the levels of IFN-γ and TNF-α using the LEGEND plex cytokine kit. (FIG. 9E) WT or Ythdf2-cKO mice were injected subcutaneously with 2×10⁶ MC38 cells. When the tumor size reached 100 mm³, mice were treated with 200 μg of CD8a-depleting antibody twice a week starting on the same day of tumor-local IR (20 Gy, one dose). Tumor growth was monitored. (FIGS. 9F-9G) Percentages of CD4⁺, CD8⁺, DCs (CD11c⁺MHCII⁺) and M-MDSCs (CD11b⁺Ly6C^{hi}) in Spleen (FIG. 9F) and lymph node (FIG. 9G) in WT and Ythdf2-cKO mice. Data are represented as mean±s.e.m., one-way ANOVA with Bonferroni's multiple comparison tests (FIGS. 9B-9D) or two-sided unpaired Student's t-test (FIGS. 9A, 9E-9G). *P<0.05. **P<0.01, and ***P<0.001.

FIGS. 10A-10C. Feature genes and proportion of each mMDSC cluster in both blood and tumors. (FIG. 10A) UMAP plot displaying different clusters of mMDSCs from blood and tumor with/without IR based on the scRNA-seq data (left). Proportion of different mMDSC cell subsets (right). (FIG. 10B) Bubble heatmap showing the expression of feature genes of each mMDSC cluster from FIG. 3C. (FIG. 10C) Bubble heatmap showing the expression of feature genes of each mMDSC cluster from FIG. 3D.

FIGS. 11A-11H. YTHDF2 affects mMDSC differentiation in the context of IR. (FIG. 11A) Density plot (left) and boxplot (right) showing pseudotime of cells within each clusters in blood with or without IR treatment. (FIG. 11B) Density plot (left) and boxplot (right) showing pseudotime of cells within each clusters in tumors with or without IR treatment. (FIG. 11C) Proportion of mMDSC cell subsets (from FIG. 3F) in blood and tumors from WT and Ythdf2-cKO mice with non-IR versus IR treatment. (FIG. 11D) Density plot (left) and boxplot (right) showing pseudotime of cells within each clusters in blood from WT and Ythdf2-cKO mice without IR treatment. (FIG. 11E) Density plot (left) and boxplot (right) showing pseudotime of cells within each clusters in blood from WT and Ythdf2-cKO mice with IR treatment. (FIG. 11F) Density plot (left) and boxplot (right) showing pseudotime of cells within each clusters in tumors from WT and Ythdf2-cKO mice without IR treatment. (FIG. 11G) Density plot (left) and boxplot (right) showing pseudotime of cells within each clusters in tumors from WT and Ythdf2-cKO mice with IR treatment. (FIG. 11H) Cell population in blood and tumors from WT or Ythdf2-cKO mice with/without IR.

FIGS. 12A-12C. Deletion of Ythdf2 in myeloid cells inhibits MDSC suppression function in the context of IR. (FIG. 12A) Percentages of MC38 tumor-infiltrating M1-like macrophages (CD45⁺CD11b⁺MHCII⁺CD206⁻; left) and PMN-MDSCs (CD45⁺CD11b⁺Ly6G⁺Ly6c⁻; right) assessed by flow cytometry (treatment conditions as indicated). (FIG. 12B) MDSCs were sorted from MC38 tumors in WT, WT+IR, Ythdf2-cKO, Ythdf2-cKO+IR mice and subjected to qPCR analysis of the mRNA level of genes as indicated. (FIG. 12C) The MC38 tumor tissues were collected in WT, WT+IR, Ythdf2-cKO and Ythdf2-cKO+IR mice to measure the protein level of IL-10 by ELISA. Data are represented as mean±s.e.m., two-tailed unpaired Student's t test) (FIG. 12A), one-way ANOVA with Bonferroni's multiple comparison tests (FIG. 12C). *P<0.05. **P<0.01.

FIGS. 13A-13D. NF-κB/RELA mediates IR-induced YTHDF2 expression in MDSCs. (FIG. 13A) Function enrichment analysis of gene signatures of Ly6c2_monocytes (P01) cluster in FIGS. 1A-1B. (FIG. 13B) Immunoblot analysis of nuclear RELA in sorted MDSCs from tumors in MC38-bearing WT mice one, two, and three days after IR (top). Immunoblot analysis of YTHDF2 in sorted MDSCs from tumors in MC38-bearing Nfkb1 knockout mice one, two, and three days after IR (bottom). (FIG. 13C) Profile of RELA binding (GSE99895) at the promoter region of Ythdf2 in bone marrow-derived macrophages. (FIG. 13D) Chromatin immunoprecipitation (ChIP) analysis of the Ythdf2 promoter in BM-MDSCs.

FIGS. 14A-14D show Transcriptome-wide analysis of m⁶A level and YTHDF2-binding sites in tumor infiltrating-CD11b⁺ myeloid cells. (FIG. 14A) Functional enrichment analysis of genes that were downregulated upon WT+IR vs. WT, and also upregulated upon Ythdf2-cKO+IR vs. WT+IR using DAVID. (FIG. 14B) Scatter plot of gene expression fold changes (log 2FoldChange) between IR versus Control, and Ythdf2-cKO+IR versus WT+IR in CD11b⁺ myeloid cells. (FIG. 14C) PCA analysis of YTHDF2 RIP-seq. (FIG. 14D) Venn diagram of YTHDF2 targets in Control and IR condition (left). Boxplot showing YTHDF2 binding intensity on its target genes in Control and IR condition (right).

FIGS. 15A-15I show YTHDF2 degrades Adrb2, Metrnl, and Smpdl3b transcripts and thereby activates NF-κB signaling in MDSCs. (FIG. 15A) MDSCs were sorted from MC38 tumors in WT, WT+IR, Ythdf2-cKO, Ythdf2-cKO+IR mice and subjected to qPCR analysis of Adrb2, Metrnl and Smpdl3b mRNA level. (FIG. 15B) Graphs showing enrichment of Adrb2, Metrnl and Smpdl3b mRNA in the YTHDF2-immunoprecipitated RNA fraction of bone marrow-derived MDSCs, determined by RIP-qPCR. (FIG. 15C) MDSCs were sorted from bone marrow-derived cells from WT and Ythdf2-cKO mice and were treated with actinomycin D. mRNA was collected at indicated time points after treatment and mRNA levels of Adrb2, Metrnl and Smpdl3b were measured by RT-qPCR. (FIG. 15D) WT, Adrb2, Metrnl and Smpdl3b knockdown MDSCs were co-cultured with LPS for 5 min. Immunoblot analysis of signaling associated proteins (as indicated) and phosphorylated (p-) proteins in these three type cells. (FIG. 15E) Bone marrow cells were transduced with a control siRNA (WT) or a siRNAs targeting Adrb2. Metrnl and Smpdl3b respectively or simultaneously (3×KD) and grown for 24-48 h. MDSCs were then purified. WT, 3×KD and 3×KD MDSCs treated with BAY 11-7082 were co-cultured with LPS for 5 min. Immunoblot analysis of nuclear RELA (P65). (FIG. 15F) Bone marrow cells from CD45.1 mice were used to generate 3×KD MDSCs using siRNA. MC38 tumor bearing Ccr2-knockout mice (CD45.2) were adoptively transferred with above purified three types of MDSCs via i.v. injection. On the same day, mice were treated with local IR (20 Gy, one dose). Three days after IR, the number of tumor-infiltrating CD45.1⁺CD11b⁺Ly6C^{hi} cells was determined. (FIG. 15G) Different MDSCs as indicated were used for the transwell assay. The attached cells on the transwell membrane were visualized under a light microscope and quantified. (FIG. 15H) WT, 3×KD, Ythdf2-cKO and 3×KD-Ythdf2-cKO MDSCs were generated from bone marrow and used for adoptive transfer into MC38 tumor bearing Ccr2-knockout mice (CD45.2). On the same day, mice were treated by with tumor-local IR (20 Gy, one dose). Three days after IR, the number of tumor-infiltrating Ccr2⁺CD11b⁺Ly6C^{hi} cells was determined. (FIG. 15I) Heatmap showing the qPCR analysis of relative Cxcl16, Ccl5, Ccl2, Ccr7, and Il10 mRNA expression in WT, 3×KD, BAY 11-7082-treated WT and BAY 11-7082-treated 3×KD MDSCs. The qPCR data were normalized to Gapdh. Data are represented as mean±s.e.m., one-way ANOVA with Bonferroni's multiple comparison tests (FIGS. 15A, 15G, 15H) or two-sided unpaired Student's t-test (FIGS. 15B, 15C, 15F). *P<0.05, **P<0.01. ***P<0.001, and ****P<0.0001.

FIGS. 16A-16E. The binding affinity, selectivity, and cell viability of compound Inhibitor B. (FIG. 16A) The MST binding curve of Inhibitor B and YTHDF2. (FIG. 16B) Inhibitory activity (IC50) of Inhibitor B against YTHDF1 binding to m⁶A detected via AlphaScreen assay. (Data are presented as the mean±SD.) (FIG. 16C) B16F1 tumor growth in WT mice with IR and/or Inhibitor B treatment. (FIG. 16D) The numbers of tumor-infiltrating CD8⁺ T cells and CD8⁺IFNγ⁺ T cells in MC38 tumor-bearing mice with treatments as indicated. (FIG. 16E) MC38 tumor growth in Rag/knockout mice with IR and/or Inhibitor B treatment. Data are represented as mean±s.e.m., two-sided unpaired Student's t-test (FIGS. 16C, 16E) or one-way ANOVA with Bonferroni's multiple comparison tests (FIG. 16D). *P<0.05, and **P<0.01.

FIGS. 17A-17I show that YHDF2 KO Tregs delay tumor development. (FIG. 17A) Ythdf2^{fl/+}/Foxp3^{cre} (WT) and Ythdf2^{fl/fl}/Foxp3^{cre} (cKO) mice were inoculated with B16F10 cells on day 0 and tumor growth was measured. 15 days after inoculation, mice were euthanized for analysis. B16F10 (FIG. 17B) or MC38 (FIG. 17C) tumors were harvested and weighted. B16F10 tumors were digested and tumor-infiltrating T cells ratios (FIG. 17D), Foxp3 expressing Treg ratio (FIG. 17E), Treg markers expression levels (FIG. 17F), Foxp3 expression level (FIG. 17G), and apoptotic Tregs (FIG. 17H) were analyzed by flow cytometry. (FIG. 17I). Transwell assay with splenic Tregs from WT and cKO mice.

FIGS. 18A-18F show that YTHDF2 regulates the survival of tumor-infiltrating Tregs and TNF signaling. Volcano plots of differentially expressed genes between WT and cKO mice splenic (FIG. 18A) or tumor (FIG. 18B) Tregs. (FIG. 18C) GO enrichment analysis of significantly downregulated genes in cKO tumor Tregs. (FIG. 18D) Heatmap of upregulated apoptotic genes expression level in WT and YTHDF2 CKO Tregs. (FIGS. 18E, 18F) GSEA of hallmark genes in TNF signaling pathway (FIG. 18E) and NF-κB negative regulators (FIG. 18F).

FIGS. 19A-19F show local tumor irradiation increases tumor-associated myeloid cells expressing YTHDF2. (FIG. 19A) UMAP plot of scRNA-seq data showing the different myeloid cell clusters of CD45⁺ immune cells, which were isolated from non-irradiated (Control) and irradiated (IR) MC38 mouse tumors, respectively (left). Bar plot showing the proportion of ‘P01:Ly6c2_Mono’ cluster in control and irradiated tumors, respectively (right). CD45⁺ immune cells were obtained from four pooled MC38 tumor-bearing mice four days after IR. (FIG. 19B) Expression levels of selected genes identifying ‘P01:Ly6c2_Mono’ cluster as MDSC in UMAP space. (FIG. 19C) Flow cytometry analysis of MDSCs in PBMCs from cancer patients with lung metastasis (pre-RT vs. post-RT). The post-RT blood samples were collected approximately 1-3 weeks (median 14 days) after the pre-RT samples. (FIG. 19D) Heatmap showing the mRNA expression of m⁶A-related genes (identified by qPCR analysis) in MDSCs from non-irradiated and irradiated MC38 tumors three days after IR. One representative result (of three independent experiments) with three technical replicates was shown. (FIG. 19E) Mean Fluorescent Intensity (MFI) of YTHDF2 in MDSCs of PBMCs (same cells used in FIG. 19C) from non-responders patients pre- and post-RT by flow cytometry. (FIG. 19F) Representative flow cytometry analysis of YTHDF2 expression in MC38 tumor-infiltrating MDSCs (CD45⁺CD11b⁺Ly6C^{hi}) three days after IR. Statistical analysis was performed using two-sided paired Student's t-test (FIGS. 19C, 19E). See also FIG. 25.

FIGS. 20A-20G show Ythdf2 deficiency in myeloid cells improves response to radiotherapy. (FIGS. 20A-20B) Wild-type (Ythdf2^{fl/fl}) or Ythdf2-cKO (Lyz^{cre}Ythdf2^{fl/fl}) mice were injected subcutaneously with 1×10⁶ MC38 cells. When the tumor size reached 100 mm³, tumor-bearing mice were treated with tumor-local IR (20 Gy, one dose). Tumor growth (FIG. 20A) and survival were monitored (FIG. 20B). Mice with tumor volumes less than 2,000 mm³ were considered to be surviving. (FIGS. 20C-20D) Wild-type or Ythdf2-cKO mice were injected subcutaneously with 1×10⁶ B16-OVA cells (FIG. 20C) or 1×10⁶ LLC cells (FIG. 20D). When the tumor size reached 100 mm³, tumor-bearing mice were treated with local IR (20 Gy, one dose). Tumor growth was monitored. (FIG. 20E) Lung metastasis in WT or Ythdf2-cKO mice 22 days after IR. Treatments as indicated in (FIG. 20D). Size of lung metastases was measured. (n=8 per group) (FIG. 20F) Populations of MC38 tumor-infiltrating immune cells assessed by flow cytometry (treatment conditions as indicated). MDSC: CD45⁺CD11b⁺Gr1⁺; Macrophages: CD45⁺CD11b⁺F4/80⁺; DCs: CD45⁺CD11c⁺MHCII⁺; CD8⁺ T: CD45⁺CD8a⁺; CD4⁺ T: CD45⁺CD4⁺; (n=3 per group) (FIG. 20G) The number (left) and percentage (right) of tumor-infiltrating CD45⁺CD11b⁺Ly6C^{hi} cells three days after IR, as assessed by flow cytometry. (n=3-5 per group) Data are represented as mean±s.e.m., n, number of mice. One of two or three representative experiments was shown. Statistical analysis was performed using two-way ANOVA test with corrections for multiple variables (FIGS. 20A, 20C, 20D), two-sided log-rank (Mantel-Cox) test (B) or one-way ANOVA with Bonferroni's multiple comparison tests (FIGS. 20E, 20G). *P<0.05, **P<0.01, and ***P<0.001.

FIGS. 21A-221I show IR and YTHDF2 inhibition reshapes the composition of MDSC populations in blood and tumors. (FIG. 21A) UMAP plot displaying different mMDSCs-derived subsets from scRNA-seq. The CD45⁺CD11b⁺Ly6C^{hi} cells were sorted from blood and tumors in IR-treated MC38 tumor-bearing mice, respectively three days after IR. (Five mice were pooled per group). (FIG. 21B) Cell proportion changes (IR vs. non-IR) of different mMDSCs-derived subsets in blood and tumors, respectively. (FIG. 21C) Cell trajectory of cell populations in blood (only including monocytes and macrophages subsets) were visualized using UMAP. (FIG. 21D) Cell trajectory of cell populations in tumors (only including monocytes and macrophages subsets) were visualized using UMAP. (FIG. 21E) Proportion of different mMDSC-derived subsets in tumors with non-IR versus IR treatment. (FIG. 21F) Proportion of mMDSC-derived subsets in blood and tumors from WT and Ythdf2-cKO mice with non-IR versus IR treatment (left); Cell proportion changes of mMDSC-derived subsets in tumors in WT+IR vs. WT and Ythdf2-cKO+IR vs. WT+IR (right). (FIG. 21G) Cell trajectory of combined cell populations in blood and tumors from WT or Ythdf2-cKO mice. (FIG. 21H) Expression level of gene signatures of C15 in UMAP space. (FIG. 21I) Proportion of C15 and C9 clusters from blood and tumors, respectively (Ythdf2-cKO+IR versus WT+IR). See also FIGS. 27, 28.

FIGS. 22A-22G show YTHDF2 controls MDSC migration and suppressive function in the context of IR. (FIG. 22A) MDSCs were sorted from MC38 tumors, as indicated in (FIG. 2A), and subjected to the trans-well migration assay. Migrated cells on the trans-well membranes were visualized under a light microscope and quantified. (n=3 per group) (FIG. 22B) MC38 tumor fragments from WT or Ythdf2-cKO mice (CD45.2) were transplanted into CD45.1 WT mice. Three days later, tumors were treated with local tumor IR (20 Gy, one dose). Three days after IR, the number of tumor-infiltrating CD45.1⁺CD11b⁺Ly6C^{hi} cells was determined by flow cytometry. (n=5 per group) (FIG. 22C) MC38 tumor fragments from CD45.1 WT mice were transplanted into WT or Ythdf2-cKO mice (CD45.2). Three days later, tumors were treated with local tumor IR (20 Gy, one dose). Three days after IR, the number of tumor-infiltrating CD45.2⁺CD11b⁺Ly6C^{hi} cells was determined by flow cytometry. (n=5 per group) (FIG. 22D) The YTHDF2 (Ythdf2-WT) and m⁶A-binding-site-mutated YTHDF2 (Ythdf2-Mut) overexpressing Ythdf2-cKO BM-MDSCs (CD45.2) were adoptive transfer into MC38 tumor-bearing CD45.1 mice. On the same day, mice were treated with local tumor IR (20 Gy). Three days after IR, the number of tumor-infiltrating CD45.2⁺CD11b⁺Ly6C^{hi} cells was determined by flow cytometry. (n=5 per group) (FIG. 22E) CD11b⁺ myeloid cells were sorted from MC38 tumors, as indicated in FIG. 2A, and subjected to bulk mRNA-seq. Heatmap of functional enrichment analysis of differentially expressed gene pathways. (FIG. 22F) Violin plot of gene expression fold changes (log 2FC) in genes related to chemokine signaling pathways, cell migration, and positive regulation of cell migration pathways (comparing WT+IR versus WT+Control, and Ythdf2-cKO+IR versus WT+IR). (FIG. 22G) Flow cytometry analysis of an in vitro proliferation assay showing the frequency of proliferating CD8⁺ T cells when co-cultured with MDSCs sorted from different MC38 tumors, as indicated. (n=3 per group). Data are represented as mean±s.e.m., n, number of mice. One of two representative experiments was shown (FIGS. 22A-22C). Statistical analysis was performed using one-way ANOVA with Bonferroni's multiple comparison tests (FIGS. 22A-22D, 22G). *P<0.05. **P<0.01. ***P<0.001, and ****P<0.0001. See also FIG. 28.

FIGS. 23A-23F show IR-induced YTHDF2 enhances NF-κB signaling by promoting m⁶A-modified RNA degradation. (FIG. 23A) The tumor-infiltrating CD11b⁺ myeloid cells were isolated from MC38 tumor-bearing WT or Ythdf2-cKO mice with IR or unirradiated controls three days after IR followed by bulk RNA-seq. Gene Set Enrichment Analysis (GSEA) of differentially expressed genes following IR treatment (IR vs. Ctrl) against ranked list of genes according to expression changes comparing Ythdf2-cKO+IR versus WT+IR. (FIG. 23B) Venn diagram of overlapping genes that were downregulated following IR vs. Ctrl and upregulated following Ythdf2-cKO+IR vs. WT+IR (top); or upregulated upon IR vs. ctrl and downregulated upon Ythdf2-cKO+IR vs. WT+IR (bottom). (FIG. 23C) Volcano plot of genes with differential expression levels in the tumor-infiltrating CD11b⁺ myeloid cells (IR vs. Ctrl). M6A marked genes are shown with orange circles. Downregulated genes (downDEGs) are highlighted with blue and upregulated genes (upDEGs) with red. CD11b⁺ myeloid cells were collected from five pooled MC38 tumor-bearing mice three days after IR. (FIG. 23D) Boxplot showing gene expression log 2FC comparing WT+IR vs. WT+ctrl (left); and Ythdf2-cKO+IR vs. WT+IR (right). Genes were categorized into two groups according to whether they were marked with m⁶A or not (non-m⁶A). For box plots, the center lines represent the medians, the box show the upper (top) and lower (bottom) quartiles, vertical lines represent 1.5× the interquartile ranges. P values were calculated by the nonparametric Wilcoxon-Mann-Whitney test. (FIG. 23E) The tumor-infiltrating CD11b⁺ myeloid cells were collected from five pooled MC38 tumor-bearing mice three days after IR followed by RIP-seq. Scatter plot of YTHDF2 binding intensity on its target genes (Ctrl vs. IR). (FIG. 23F) Heatmap showing gene expression level in WT mice with non-IR (WT+Ctrl) and IR (WT+IR) treatment, and Ythdf2-cKO mice with IR treatment (cKO+IR) (left). Genes were further categorized into groups according to whether they were bound by YTHDF2, or marked with m⁶A (right). See also FIG. 29.

FIGS. 24A-24E show pharmacological inhibition of YTHDF2 enhances responses to radiotherapy and immunotherapy. (FIGS. 24A-24B) WT mice were injected subcutaneously with 1×10⁶ MC38 cells (FIG. 24A) or 1×10⁶ B16F1 cells (FIG. 24B). When the tumor size reached 100 mm³, tumors were treated with local IR (20 Gy, one dose). On the same day, the mice were treated with Inhibitor B (9 μg/per mice, daily) until the end of the experiment. Tumor growth was monitored. (FIG. 24C) WT mice were injected subcutaneously with 1×10⁶ MC38 cells. When the tumor size reached 100 mm³, tumors were treated with local IR (20 Gy, one dose), anti-PD-L1 antibody (2 doses per week, three doses total) and/or Inhibitor B (9 μg/per mice, daily), as indicated. Tumor growth was monitored. (FIG. 24D) The numbers of tumor-infiltrating CD45⁺CD11b⁺Ly6C^{hi} cells in mice, with different treatment as indicated, were assessed by flow cytometry three days after IR. (n=5 per group) (FIG. 24E) The numbers of tumor-infiltrating CD45⁺CD8⁺IFNγ⁺ T cells in MC38 tumor-bearing mice with treatments as indicated seven days after IR. (n=5 per group) Data are represented as mean±s.e.m., n, number of mice. One of two representative experiments was shown. Statistical analysis was performed using two-way ANOVA test with corrections for multiple variables (A-C) or one-way ANOVA with Bonferroni's multiple comparison tests (D, E). *P<0.05 and **P<0.01. See also FIG. 30.

FIGS. 25A-25K show scRNA-seq identified CD45⁺ immune cell populations and YTHDF2 expression in IR-treated MDSCs. Related to FIG. 19. (FIGA. 25A-25B) UMAP showing five clusters including T cells, NK, Macrophages, DCs and Monocytes (FIG. 25A) from scRNA-seq using sorted CD45⁺ immune cells from MC38 tumors with and without IR treatment, respectively (FIG. 25B) four days after IR. (FIG. 25C) Proportion of different cell subsets of CD45⁺ immune cells (as in Figure S1A-B) in irradiated vs. non-irradiated MC38 tumors. (FIG. 25D) Changes in proportion of different cell subsets of CD45⁺ immune cells (as in FIG. S1C) in irradiated vs. non-irradiated MC38 tumors. (FIG. 25E) Bubble heatmap showing the expression of feature genes of each myeloid cells cluster from FIG. 1A. (FIG. 25F) Proportion of different cell subsets of myeloid cells (as in FIG. 1A) in irradiated vs. non-irradiated MC38 tumors. (FIG. 25G) Flow cytometry analysis of MDSCs in PBMCs from metastatic NSCLC patients enrolled in a clinical trial (the COSINR study, NCT03223155) (pre-RT vs. post-RT) and Mean Fluorescent Intensity (MFI) of YTHDF2 in MDSCs of PBMC from non-responders patients. Non-responder was characterized as <8 month to ‘progression or death’ (the average progression time), while responder was characterized as with >8 months to ‘progression or death’. (FIG. 25H) Overall survival analysis of cancer patients in Low Grade Glioma (LGG), and Glioblastoma (GBM) cohorts and either high or low MDSC signature. Normalized gene expression and corresponding clinical data on patients were obtained from TCGA. (FIG. 25I) Flow cytometric analysis of YTHDF2 expression in different immune cells (as indicated) isolated from irradiated MC38 tumors. Macrophages: CD11b⁺F4/80⁺; mMDSC: CD11b⁺Ly6C^{hi} Ly6G⁻; PMN-MDSC: CD11b⁺Ly6G⁺Ly6C⁻; DCs: CD11c⁺MHCII⁺; CD4⁺ T: CD45⁺CD4⁺; CD8⁺ T: CD45⁺CD8a⁺; (n=4-5 per group). (FIG. 25J) MDSCs sorted from tumors in MC38-bearing WT mice one, two, and three days after IR. Immunoblot analysis of YTHDF2 in sorted MDSCs. (FIG. 25K) MDSCs were derived from bone marrow cells (WT mice) and treated with different doses of IR directly and cultured for different times as indicated. Graph showing the immunoblot analysis of YTHDF2. Data are represented as mean±s.e.m., Statistical analysis was performed using two-sided paired Student's t-test (G) or two-sided unpaired Student's t-test (I). *P<0.05 and **P<0.01.

FIGS. 26A-26G show the antitumor effects of IR in Ythdf2-cKO mice depend on CD8⁺ T cell response. Related to FIG. 20. (FIGS. 26A-26B) The numbers of MC38 tumor-infiltrating total CD8⁺ T cells (FIG. 26A) and CD8⁺IFNγ⁺ T cells (FIG. 26B) in WT or Ythdf2-cKO mice with/without IR (7 days after IR). (n=5 per group) (FIG. 26C) The CD8⁺ T cells were isolated from MC38 tumors (in A) and the IFN-γ spots were enumerated by ELISPOT assay. (FIG. 26D) The MC38 tumor tissues (in A) were collected to measure the levels of IFN-γ and TNF-α using the LEGEND plex cytokine kit. (FIG. 26E) WT or Ythdf2-cKO mice were injected subcutaneously with 1×10⁶ MC38 cells. When the tumor size reached 100 mm³, mice were treated with 200 μg of CD8a-depleting antibody twice a week starting on the same day of tumor-local IR (20 Gy, one dose). Tumor growth was monitored. (n=5 per group). (FIGS. 26F-26G) Percentages of CD4⁺, CD8⁺, DCs (CD11c⁺MHCII⁺) and mMDSCs (CD11b⁺Ly6C^{hi} Ly6G⁻) in Spleen (FIG. 26F) and lymph node (FIG. 26G) in WT and Ythdf2-cKO mice. (n=4-6 per group) Data are represented as mean±s.e.m., n, number of mice. One of two representative experiments was shown. Statistical analysis was performed using one-way ANOVA with Bonferroni's multiple comparison tests (FIGS. 26A-26D), two-way ANOVA test with corrections for multiple variables (FIG. 26E), or two-sided unpaired Student's t-test (F, G). *P<0.05, **P<0.01, and ***P<0.001.

FIGS. 27A-27L show IR or YTHDF2 affects mMDSC differentiation in both blood and tumors. Related to FIG. 21. (FIG. 27A) UMAP plot displaying different mMDSC-derived subsets from blood and tumor with/without IR based on the scRNA-seq data of tumor-infiltrating CD45⁺CD11b⁺Ly6C^{hi} cells (upper). Proportion of different mMDSC-derived subsets (lower). The cells were sorted from blood or tumors in five pooled IR-treated MC38 tumor-bearing mice three days after IR. (FIG. 27B) Bubble heatmap showing the expression of feature genes of each mMDSC-derived cluster in blood from FIG. 21C. (FIG. 27C) Bubble heatmap showing the expression of feature genes of each mMDSC-derived cluster in tumor from FIG. 21D. (FIG. 27D) Density plot (left) and boxplot (right) showing pseudotime of cells within each cluster in blood with or without IR treatment. (FIG. 27E) Density plot (left) and boxplot (right) showing pseudotime of cells within each cluster in tumors with or without IR treatment. (FIG. 27F) Cell population in blood and tumors from WT or Ythdf2-cKO mice with/without IR. (FIG. 27G) Proportion of mMDSC-derived subsets (from FIG. 3F) in blood and tumors from WT and Ythdf2-cKO mice with non-IR versus IR treatment. (FIG. 27H) Density plot (left) and boxplot (right) showing pseudotime of cells within each cluster in blood from WT and Ythdf2-cKO mice without IR treatment. (FIG. 27I) Density plot (left) and boxplot (right) showing pseudotime of cells within each cluster in blood from WT and Ythdf2-cKO mice with IR treatment. (FIG. 27J) Density plot (left) and boxplot (right) showing pseudotime of cells within each cluster in tumors from WT and Ythdf2-cKO mice without IR treatment. (FIG. 27K) Density plot (left) and boxplot (right) showing pseudotime of cells within each cluster in tumors from WT and Ythdf2-cKO mice with IR treatment. (FIG. 27L) (Upper) UMAP plot displaying CD45⁺CD11b⁺Ly6C^{hi} cells in blood and tumors from WT or Ythdf2-cKO mice with/without IR. Cells were colored according to which tissue they belong to. (Lower) A barplot illustrating the ratio of the number of cells from blood to the number of cells from tumor in each cluster. The clusters were ranked based on the ratio. For box plots (D, E, H-K), the center line represents the median, the box limits show the upper and lower quartiles, whiskers represent 1.5× the interquartile range. P values were calculated by a nonparametric Wilcoxon-Mann-Whitney test. *P<0.05, **P<0.01. ***P<0.001, ****P<0.0001.

FIGS. 28A-28G show Ythdf2 deletion inhibits MDSC suppression function and NF-κB/RELA mediates IR-induced YTHDF2 expression in MDSCs. Related to FIGS. 3 and 4. (FIG. 28A) Percentages of MC38 tumor-infiltrating M1-like macrophages (CD45⁺CD11b⁺MHCII⁺CD206⁻; left) and PMN-MDSCs (CD45⁺CD11b⁺Ly6G⁺Ly6C⁻; right) assessed by flow cytometry (treatment conditions as indicated). (n=3-4 per group) (FIG. 28B) MDSCs were sorted from MC38 tumors in WT. WT+IR, Ythdf2-cKO, Ythdf2-cKO+IR mice three days after IR and subjected to qPCR analysis of the mRNA level of genes as indicated. (n=3 per group) (FIG. 28C) The MC38 tumor tissues were collected in WT, WT+IR, Ythdf2-cKO and Ythdf2-cKO+IR mice to measure the protein level of IL-10 by ELISA. (n=5 per group) (FIG. 28D) Function enrichment analysis of gene signatures of Ly6c2_monocytes (P01) cluster in FIG. 1A. (FIG. 28E) Immunoblot analysis of nuclear RELA in sorted MDSCs from tumors in MC38-bearing WT mice one, two, and three days after IR (top). Immunoblot analysis of YTHDF2 in sorted MDSCs from tumors in MC38-bearing Nfkb1 knockout mice one, two, and three days after IR (bottom). (FIG. 28F) Profile of RELA binding (GSE99895) at the promoter region of Ythdf2 in bone marrow-derived macrophages. (FIG. 28G) Chromatin immunoprecipitation (ChIP) analysis of the Ythdf2 promoter in BM-MDSCs. Data are represented as mean±s.e.m., n, number of mice. Statistical analysis was performed using two-tailed unpaired Student's t test (FIG. 28A), one-way ANOVA with Bonferroni's multiple comparison tests (C). *P<0.05. **P<0.01.

FIGS. 29A-29M show YTHDF2 degrades Adrb2, Metrnl, and Smpdl3b transcripts and thereby activates NF-κB signaling in MDSCs. Related to FIG. 23. (FIG. 29A) Functional enrichment analysis of genes in tumor-infiltrating CD11b⁺ myeloid cells that were downregulated upon WT+IR vs. WT, and also upregulated upon Ythdf2-cKO+IR vs. WT+IR using DAVID. (FIG. 29B) Scatter plot of gene expression fold changes (log 2FC) between IR versus Control, and Ythdf2-cKO+IR versus WT+IR in CD11b⁺ myeloid cells. (FIG. 29C) PCA analysis of YTHDF2 RIP-seq (as in FIG. 5E). (FIG. 29D) Venn diagram of YTHDF2 targets (from YTHDF2 RIP-seq data) in Control and IR condition (left). Boxplot showing YTHDF2 binding intensity on its target genes in Control and IR condition (right). For box plots, the center line represents the median, the box limits show the upper and lower quartiles, whiskers represent 1.5× the interquartile range. P values were calculated by a nonparametric Wilcoxon-Mann-Whitney test. (FIG. 29E) MDSCs were sorted from MC38 tumors in WT, WT+IR, Ythdf2-cKO, Ythdf2-cKO+IR mice and subjected to qPCR analysis of Adrb2, Metrnl and Smpdl3b mRNA level. (n=3 per group). (FIG. 29F) Graphs showing enrichment of Adrb2. Metrnl and Smpdl3b mRNA in the YTHDF2-immunoprecipitated RNA fraction of bone marrow-derived MDSCs, determined by RIP-qPCR. (FIG. 29G) MDSCs were sorted from bone marrow-derived cells from WT and Ythdf2-cKO mice and were treated with actinomycin D. mRNA was collected at indicated time points after treatment and mRNA levels of Adrb2, Metrnl and Smpdl3b were measured by RT-qPCR. (n=3 per group). (FIG. 29H) BM-MDSCs were transduced with siRNAs targeting Adrb2. Metrnl and Smpdl3b simultaneously (3×KD). After 24-48 hr, the BM-MDSCs were purified and subjected to qPCR analysis of these three genes. (n=3 per group). (FIG. 29I) (left) WT. Adrb2, Metrnl and Smpdl3b knockdown MDSCs were co-cultured with LPS for 5 min. Immunoblot analysis of signaling associated proteins (as indicated) and phosphorylated (p-) proteins in these three type cells. (right) WT, 3×KD BM-MDSCs and BAY 11-7082 treated 3×KD BM-MDSCs (for 24 hr) were co-cultured with LPS for 5 min. Immunoblot analysis of nuclear RELA. (FIG. 29J) Bone marrow cells from CD45.1 mice were used to generate 3×KD BM-MDSCs using siRNA. MC38 tumor bearing Ccr2-knockout mice (CD45.2) were adoptively transferred with 1×10⁶ WT or 3×KD BM-MDSCs via i.v. injection. On the same day, mice were treated with local IR (20 Gy, one dose). Three days after IR, the number of tumor-infiltrating CD45.1⁺CD11b⁺Ly6C^{hi} Ly6G⁻ cells was determined by flow. (n=6 per group). (FIG. 29K) Different BM-MDSCs as indicated were used for the transwell assay. The attached cells on the transwell membrane were visualized under a light microscope and quantified. (n=3 per group). (FIG. 29L) WT, 3×KD, Ythdf2-cKO and 3×KD-Ythdf2-cKO BM-MDSCs were used for adoptive transfer into MC38 tumor bearing Ccr2-knockout mice. On the same day, mice were treated with tumor-local IR (20 Gy, one dose). Three days after IR, the number of tumor-infiltrating Ccr2⁺CD11b⁺Ly6C^{hi} cells was determined by flow. (n=4 per group). (FIG. 29M) Heatmap showing the qPCR analysis of relative Cxcl16, Ccl5, Ccl2, Ccr7, and Il10 mRNA expression in WT. 3×KD, BAY 11-7082-treated WT and BAY 11-7082-treated 3×KD BM-MDSCs. The qPCR data were normalized to Gapdh. (n=3 per group). Data are represented as mean±s.e.m., n, number of mice. One of two or three representative experiments was shown (FIGS. 29E-29M). Statistical analysis was performed using one-way ANOVA with Bonferroni's multiple comparison tests (FIGS. 29E, 29K, 29L) or two-sided unpaired Student's t-test (FIGS. 29F, 29G, 29H, 29J). *P<0.05. **P<0.01. ***P<0.001, and ****P<0.0001.

FIGS. 30A-30I show The binding affinity, and selectivity of compound Inhibitor B. Related to FIG. 24. (FIG. 30A) Inhibitory activities (IC₅₀) of Inhibitor A (red) and Inhibitor B (blue) against YTHDF2 binding to m⁶A determined by AlphaScreen assay (right). Data are presented as mean±SD. (FIG. 30B) The MST binding curve of Inhibitor B and YTHDF2. Data are presented as mean±SD. (FIG. 30C) SPR binding curves of Inhibitor B and YTHDF2. The concentrations of the different compounds injected over the CM5 chip are indicated. Data are represented as mean±s.e.m., one of three independent experiments is shown. (FIG. 30D) (left) Hela cells were transduced with siRNA targeting YTHDF2 and subjected to qPCR analysis of YTHDF2 and its target gene PRR5. (right) Hela cells were treated with Inhibitor B in different concentration as indicated and subjected to qPCR analysis of PRR5 (a target gene of YTHDF2 in Hela cells). Data are presented as the mean±SD. (FIG. 30E) Inhibitory activity (IC50) of Inhibitor B against YTHDF1 binding to m⁶A detected via AlphaScreen assay. Data are presented as the mean±SD. (FIG. 30F) Immunoblot analysis of Hela cells treated with siRNA targeting YTHDF1 or Inhibitor B to detect the protein levels of YTHDF1 and its target LRPAP1. (FIG. 30G) Immunoblot analysis of nuclear RELA in BM-MDSCs treated with Inhibitor B (for 24 hr) in different dose as indicated. (FIG. 30H) The numbers of tumor-infiltrating CD8⁺ T cells in MC38 tumor-bearing mice with treatments as indicated. (Seven days after IR, n=5 per group). Data are represented as mean±s.e.m. (FIG. 30I) MC38 tumor growth in Rag1 knockout mice with IR and/or Inhibitor B treatment. (n=5 per group). Data are represented as mean±s.e.m. Statistical analysis was performed using two-sided unpaired Student's t-test (D), one-way ANOVA with Bonferroni's multiple comparison tests (H), or two-way ANOVA test with corrections for multiple variables (I). *P<0.05.

FIG. 31 shows a graphical illustration of aspects disclosed herein.

### DETAILED DESCRIPTION

The present disclosure is based, at least in part, on the surprising discovery that the m⁶A reader YTHDF2 suppresses antitumor immunity by promoting immunosuppression following radiotherapy and/or immune checkpoint blockade. As disclosed herein, the m⁶A reader YTHDF2 suppresses antitumor immunity by promoting immunosuppression following radiotherapy and/or immune checkpoint blockade via regulating MDSC migration and function. The mechanism of YTHDF2 immunosuppression occurs, in some aspects, by modulating expression of three negative regulators of NF-κB signaling; YTHDF2 was rapidly induced via IR-activated NF-κB/RelA, implying the relationship between YTHDF2 expression and radiotherapy. YTHDF2 triggers degradation of Adrb2, Metrnl and Smpdl3b, whose gene products are the negative regulator to IκBα, leading to enhanced NF-κB signaling, resulting a positive feedback to sustain YTHDF2 expression. Thus, the present disclosure is also based, at least in part, on the surprising discovery that the IR (stress)-YTHDF2-NF-κB circuit elicits MDSC migration and suppression function, which enhances extrinsic radioresistance.

Further, administering a cancer therapy and a YTHDF2 inhibitor was surprisingly found to decrease immunosuppression of a systemic response to a cancer therapy treatment. As disclosed herein, administration of a highly effective YTHDF2 inhibitor suppressed MDSCs migration and function. This pharmacological blockade of YTHDF2 resulted in superior antitumor effects of radiotherapy, immune blockade immunotherapy, or the synergistic combination of IR and immune blockade in a CD8⁺ T cell dependent manner. In some aspects, YTHDF2 inhibitors complement and synergize with existing cancer therapies to overcome barriers of suppressive cells, to improve the adaptive immune response and for enhanced efficacy and response rates. These results demonstrate a surprising regulatory role of YTHDF2 in myeloid cells that may result, in some aspects, in resistance to cancer therapies. In some aspects, by targeting YTHDF2, a potential therapeutic strategy for effective cancer treatment is provided.

Accordingly, in some aspects, disclosed are methods and compositions for treating cancer comprising administering a YTHDF2 inhibitor and a cancer therapy (e.g., immunotherapy and/or radiotherapy) to a subject having or suspected or having cancer. In some aspects, the subject has been diagnosed with or is suspected of having cancer. In some aspects, the disclosed methods comprise providing the YTHDF2 inhibitor and the cancer therapy to a subject who was previously treated for cancer and who was determined to be resistant to the previous treatment. In some aspects, the previous cancer treatment comprised a radiotherapy and/or an immunotherapy. In some aspects, the subject has or has previously had resistance to radiotherapy or immunotherapy. In some aspects, administration of the YTHDF2 inhibitor decreases suppression of an immune response to the cancer therapy. In some aspects, administration of the YTHDF2 inhibitor inhibits NF-κB signaling in myeloid-derived suppressor cells in the subject. In some aspects, administration of the YTHDF2 inhibitor decreases myeloid-derived suppressor cell trafficking and function in the subject.

The disclosure is also based, in part, on the discovery that ionizing radiation (IR) mediated an increase of YTHDF2-expressing MDSC population following radiation therapy (RT), which was associated with metastasis progression post RT. In murine models, loss of Ythdf2 in myeloid cells augmented the efficacy of local tumor IR by altering MDSC differentiation, inhibiting MDSC trafficking into tumors, and attenuating their suppressive functions. The induction of YTHDF2 by IR via NF-kB activation resulted in downregulation of its direct targets Adrb2, Metrnl, and Smpdl3b, which negatively regulate NF-kB signaling. The YTHDF2 inhibitor enhanced the antitumor effects of radiotherapy and radio-immunotherapy combinations in a manner similar to the deletion of YTHDF2. The alleviation of immunosuppression through YTHDF2 blockade is a therapeutic paradigm that not only improves local tumor control but also suppresses distant metastasis.

The disclosure is also based, in part, on the discovery that ionizing radiation (IR) induces immunosuppressive myeloid-derived suppressor cell (MDSC) expansion and YTHDF2 expression in both murine models and humans. Following IR, loss of Ythdf2 in myeloid cells augments antitumor immunity and overcomes tumor radioresistance by altering MDSC differentiation, and inhibiting MDSC infiltration and suppressive function. The remodeling of the landscape of MDSC populations by local IR is reversed by Ythdf2 deficiency. IR-induced YTHDF2 expression relies on NF-κB signaling; YTHDF2 in turn leads to NF-κB activation by directly binding and degrading transcripts encoding negative regulators of NF-κB signaling, resulting in an IR-YTHDF2-NF-κB circuit. Pharmacological inhibition of YTHDF2 overcomes MDSC-induced immunosuppression and improves combined IR and/or anti-PD-L1 treatment.

### I. Therapeutic and Diagnostic Methods

Aspects of the disclosure are directed to compositions and methods for therapeutic use. The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration. Aspects of the disclosure are also directed at diagnostic methods for determining cancer aggressiveness, progression, metastasis, or other clinically relevant information.

### A. Cancer Therapies and Diagnostics

In some aspects, the disclosed methods comprise administering a cancer therapy to a subject or patient. The cancer therapy may be chosen based on expression level measurements, alone or in combination with a clinical risk score calculated for the subject. In some aspects, the cancer therapy comprises a local cancer therapy. In some aspects, the cancer therapy excludes a systemic cancer therapy. In some aspects, the cancer therapy excludes a local therapy. In some aspects, the cancer therapy comprises a local cancer therapy without the administration of a systemic cancer therapy. In some aspects, the cancer therapy comprises an immunotherapy, which may be an immune blockade or immune checkpoint inhibitor therapy. In some aspects, the cancer therapy comprises radiotherapy. Any of these cancer therapies may also be excluded. Combinations of these therapies may also be administered. For example, a cancer therapy may comprise a combination of an immunotherapy and radiotherapy.

Also disclosed are methods comprising measuring the level of certain gene products in a cancer patient and/or measuring the level of certain gene products in a subject having, suspected of having, or diagnosed with having cancer.

The term “cancer,” as used herein, may be used to describe a solid tumor, metastatic cancer, or non-metastatic cancer. In certain aspects, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia.

### B. YTHDF2 Inhibitors

In some aspects, the disclosed methods comprise administering a cancer therapy and a YTHDF2 inhibitor to a subject or patient. YTHDF2 (also known as YTH N⁶-methyladenosine RNA binding protein 2. YT521-B homology domain family 2, HGRG8, NY-REN-2, CAHL, or DF2) is a protein that in humans is encoded by the YTHDF2 gene. YTHDF2 is a member of m⁶A “readers.” The m⁶A modification is the methylation of the N⁶ position of adenosine bases and is the most common internal RNA modification in eukaryotic cells. m⁶A RNA methylation is enriched in 3′ untranslated regions and functions to modify kinds of RNAs, such as microRNAs (miRNAs), long non-coding RNAs (lncRNAs), and messenger RNAs (mRNAs). Additionally, YTHDF2 influences various aspects of RNA metabolism, including mRNA decay and pre-ribosomal RNA (pre-rRNA) processing. YTHDF2 activity is involved in multiple biological processes, such as migration, invasion, metastasis, proliferation, apoptosis, cell cycle, cell viability, cell adhesion, differentiation and inflammation, in both human cancers and non-cancers by modulating, e.g., miR-403-3p, SETD7, KLF4, SOCS2, OCT4, IL11, SERPINE2, MEK, ERK, EGFR, CDK1, WEE1, circ0001105, PER1 and ATM-CHK2-P53/CDC25C. In some aspects, YTHDF2 participates in the development of various cancers, including but not limited to, e.g., bladder cancer, hepatocellular carcinoma (HCC), gastric cancer, breast cancer, osteosarcoma, cervical cancer, prostate cancer, pancreatic cancer, acute myeloid leukemia (AML), etc. In some aspects, as described herein, YTHDF2 predicts the prognosis of different cancers. For example, up-regulated YTHDF2 indicated a poor prognosis in patients with cervical cancer, and down-regulated YTHDF2 predicted more aggressive tumor phenotypes and a worse prognosis of osteosarcoma.

As used herein, a “YTHDF2 inhibitor” describes any compound capable of reducing or eliminating binding of YTHDF2 to a m⁶A or m⁶A-containing nucleic acid in a cell.

### 1. Oligonucleotide YTHDF2 Inhibitors

In some aspects, a YTHDF2 inhibitor is a nucleic acid capable of binding to a region of a YTHDF2 messenger RNA. In some aspects, a YTHDF2 inhibitor is a nucleic acid capable of binding to a region of a YTHDF2 gene or a complement thereof. In some aspects, a YTHDF2 inhibitor is a nucleic acid that inhibits interaction of YTHDF2 protein with m⁶A-containing mRNA. Examples of YTHDF2 inhibitors of the present disclosure include, but are not limited to, oligonucleotides, e.g., antisense oligonucleotides, small inhibitory RNAs, small hairpin RNAs; viral vector; or guide RNAs. In some aspects, the YTHDF2 inhibitor is an oligonucleotide targeting YTHDF2 mRNA. In some aspects, the YTHDF2 inhibitor is a YTHDF2-targeting siRNA, shRNA, or antisense oligonucleotide.

“Oligonucleotide” refers to a plurality of joined nucleotide units formed in a specific sequence from naturally occurring bases and pentofuranosyl groups joined through a sugar group by native phosphodiester bonds. This term refers to both naturally occurring and synthetic species formed from naturally occurring subunits.

In some aspects, the oligonucleotide may be a modified oligonucleotide that has non-naturally occurring portions. Modified oligonucleotide can have altered sugar moieties, altered base moieties or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Modified oligonucleotides can include modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true nucleic acid species. All such compounds are considered to be analogs.

The modified oligonucleotides may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH₂—RA-NR₁ CH₂, CH₂—NR₁—RA--CH₂, RA--NR₁—CH₂—, CH₂—CH₂—NR₁—RA, CH₂—CH₂—RA--NR₁, or NR₁—RA--CH₂—CH₂ where RA is O or NR₂.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Antisense oligonucleotides of the disclosure may be at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides (or any derivable range therein). In some aspects, oligonucleotides of the disclosure include a flanking sequence. Several types of flanking sequences may be used. In some aspects, flanking sequences are used to modify the binding of a protein to said molecule or oligonucleotide, or to modify a thermodynamic property of the oligonucleotide, or to modify target RNA binding affinity.

In some aspects, an oligonucleotide of the present disclosure comprises a nucleotide-based or nucleotide or an antisense oligonucleotide sequence of between 3 and 30 nucleotides or bases, between 5 and 25 nucleotides, or between 10 and 20 nucleotides, such as 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, or 20 nucleotides.

A nucleotide sequence of an oligonucleotide of the disclosure may contain a RNA residue, a DNA residue, a nucleotide analogue or equivalent as will be further detailed herein. In some aspects, the oligonucleotide comprises at least one residue comprising a modified base, and/or a modified backbone, and/or a non-natural internucleoside linkage, or a combination of these modifications.

In some aspects, the oligonucleotide comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

In some aspects, the modified oligonucleotide comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

In some aspects, the modified oligonucleotide comprises a morpholino nucleotide analog or equivalent, in which the ribose or deoxyribose sugar is replaced by a 6-membered morpholino ring. In some aspects, the modified oligonucleotide comprises phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In some aspects, the modified oligonucleotide comprises a substitution of at least one of the non-bridging oxygens in the phosphodiester linkage. This modification slightly destabilizes base-pairing but adds significant resistance to nuclease degradation. In some aspects, the modified oligonucleotide comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

In some aspects, the modified oligonucleotide comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminocthoxyethoxy. The sugar moiety can be a pyranose or derivative thereof, or a deoxypyranose or derivative thereof, preferably a ribose or a derivative thereof, or deoxyribose or derivative thereof. In some aspects, the modified oligonucleotide comprises Locked Nucleic Acid (LNA), in which the 2′-carbon atom is linked to the 3′ or 4′ carbon atom of the sugar ring thereby forming a bicyclic sugar moiety. In some aspects, the LNA comprises 2′-O,4′-C-ethylene-bridged nucleic acid (Morita et al. 2001. Nucleic Acid Res Supplement No. 1:241-242).

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain aspects, an antisense oligonucleotide of the disclosure has at least two different types of analogues or equivalents.

In some aspects, the modified oligonucleotide comprises a 2′-O-alkyl phosphorothioate antisense oligonucleotide, such as 2′-O-methyl modified ribose (RNA), 2′-O-ethyl modified ribose, 2′-O-propyl modified ribose, and/or substituted derivatives of these modifications such as halogenated derivatives. In some aspects, the modified oligonucleotide comprises a 2′-O-methyl phosphorothioate ribose.

A functional equivalent of a molecule of the disclosure may be defined as an oligonucleotide as defined herein wherein an activity of said functional equivalent is retained to at least some extent. Preferably, an activity of said functional equivalent is reducing or eliminating YTHDF2 expression in a cell. In some aspects, an activity of said functional equivalent comprises an ability to bind to a YTHDF2 mRNA. Said activity of said functional equivalent therefore may be assessed by detection of binding to YTHDF2 mRNA and/or detection of a reduction or elimination of YTHDF2 expression in a cell. In some aspects, an activity of said functional equivalent comprises an ability to bind to inhibit interaction of YTHDF2 protein with m⁶A-containing mRNA. Said activity of said functional equivalent therefore may be assessed by detection of YTHDF2 binding to m⁶A-containing mRNA and/or detection of a reduction or elimination of protein expressed from m⁶A-containing mRNA in a cell.

An antisense oligonucleotide can be linked to a moiety that enhances uptake of the antisense oligonucleotide in cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain. In some aspects, the oligonucleotide comprises a peptide-linked PMO.

Inhibitors of the m6A mRNA modification reader may be any selected from the group consisting of: (inhibitors of HNRNPC) hsa-let-7e-5p (MIRT051596), hsa-mir-455-3p (MIRT037890), hsa-mir-30c-5p (MIRT047904), hsa-mir-186-5p (MIRT045150), hsa-mir-744-5p (MIRT037494), hsa-mir-18a-3p (MIRT040851), hsa-mir-484 (MIRT042196), hsa-mir-505-5p (MIRT037959), hsa-mir-615-3p (MIRT039991), hsa-mir-342-3p (MIRT043694), hsa-miR-3607-3p, hsa-miR-30d, hsa-miR-3916, hsa-miR-3162-5p, hsa-miR-1273d, hsa-miR-3161, hsa-miR-30a, hsa-miR-629, hsa-miR-208b, hsa-miR-489, hsa-miR-3148, hsa-miR-2113, hsa-miR-877, hsa-miR-455-5p, hsa-miR-186, hsa-miR-5480, hsa-miR-3139, hsa-miR-320a, hsa-miR-4311, hsa-miR-555, hsa-miR-3605-5p, hsa-miR-515-5p, hsa-miR-144, hsa-miR-499-5p, hsa-miR-1323, hsa-miR-548x, hsa-miR-299-5p, hsa-miR-653, hsa-miR-576-5p, hsa-miR-548p, hsa-miR-586, hsa-miR-888, hsa-miR-3647-3p, hsa-miR-484, hsa-miR-320b, hsa-miR-620, hsa-miR-30b, hsa-miR-548q, hsa-miR-29b-1, hsa-miR-570, hsa-miR-183, hsa-miR-1276, hsa-miR-208a, hsa-miR-186, hsa-miR-28-5p, hsa-miR-330-3p, hsa-miR-548 am, hsa-miR-320d, hsa-miR-3175, hsa-miR-3155, hsa-miR-548aa, hsa-miR-519c, hsa-miR-1270, hsa-miR-513b, hsa-miR-599, hsa-miR-518f, hsa-miR-4301, hsa-miR-30c, hsa-miR-3135, hsa-miR-4286, hsa-miR-202, hsa-miR-4263, hsa-miR-4299, hsa-miR-606, hsa-miR-3133, hsa-miR-583, hsa-miR-3125, hsa-miR-501-5p, hsa-miR-7-1, hsa-miR-514b-3p, hsa-miR-3155b, hsa-miR-548d-3p, hsa-miR-224, hsa-miR-7-2, hsa-miR-708, hsa-miR-3199, hsa-miR-514, hsa-miR-30e (sec. e.g. Helwak et al. Cell, 153(3): 654-655, 2013; Whisnant et al., M Bio 4(2), 2013: e000193); (inhibitors of HNRNPA2B1) hsa-mir-92a-3p (MIRT049721), hsa-mir-30c-5p (MIRT048009), hsa-mir-191-5p (MIRT045809), hsa-let-7f-5p (MIRT051404), hsa-mir-27b-3p (MIRT046213), hsa-mir-877-3p (MIRT037116), hsa-mir-615-3p (MIRT040278), hsa-mir-1260b (MIRT052680), hsa-mir-103a-3p (MIRT027027), hsa-mir-16-5p (MIRT031508), hsa-mir-1296-5p (MIRT036075), hsa-mir-197-3p (MIRT048098), hsa-miR-548j, hsa-miR-3678-3p, hsa-miR-607, hsa-miR-188-5p, hsa-miR-15a, hsa-miR-3653, hsa-miR-371-5p, hsa-miR-550a, hsa-miR-3622b-3p, hsa-miR-548a-5p, hsa-miR-3170, hsa-miR-3148, hsa-miR-556-3p, hsa-miR-490-3p, hsa-miR-559, hsa-miR-200c, hsa-miR-130a, hsa-miR-548y, hsa-miR-5480, hsa-miR-23c, hsa-miR-491-3p, hsa-miR-335, hsa-miR-3667-3p, hsa-miR-466, hsa-miR-23b, hsa-miR-4310, hsa-miR-127-5p, hsa-miR-548b-5p, hsa-miR-616, hsa-miR-16, hsa-miR-338-3p, hsa-miR-3200-5p, hsa-miR-362-3p, hsa-miR-448, hsa-miR-1306, hsa-miR-944, hsa-miR-3684, hsa-miR-373, hsa-miR-103a, hsa-miR-380, hsa-miR-499-5p, hsa-miR-1323, hsa-miR-323-5p, hsa-miR-3674, hsa-miR-1252, hsa-miR-33b, hsa-miR-580, hsa-miR-548c-3p, hsa-miR-103a-2, hsa-miR-548w, hsa-miR-600, hsa-miR-634, hsa-miR-586, hsa-miR-497, hsa-miR-720, hsa-miR-654-3p, hsa-miR-524-5p, hsa-miR-543, hsa-miR-548q, hsa-Iet-7f-2, hsa-miR-330-5p, hsa-miR-500a, hsa-miR-548l, hsa-miR-570, hsa-miR-374a, hsa-miR-1184, hsa-miR-649, hsa-miR-424, hsa-miR-3658, hsa-miR-186, hsa-miR-326, hsa-miR-548d-5p, hsa-miR-23a, hsa-miR-15b, hsa-miR-190, hsa-miR-203, hsa-miR-548h, hsa-miR-3136-5p, hsa-miR-618, hsa-miR-551b, hsa-miR-211, hsa-miR-1305, hsa-miR-513b, hsa-miR-96, hsa-miR-2117, hsa-miR-548n, hsa-miR-3910, hsa-miR-217, hsa-miR-892b, hsa-miR-502-5p, hsa-miR-548i, hsa-miR-520d-5p, hsa-miR-4299, hsa-miR-1285, hsa-miR-3133, hsa-miR-483-3p (sec. e.g., Hafner et al. Cell. 141(1): 129-141, 2010; Helwak et al. Cell, 153(3): 654-655, 2013); (inhibitors of Ythdf1) hsa-miR-548g. hsa-miR-204, hsa-miR-3143, hsa-miR-521, hsa-miR-195, hsa-miR-3182, hsa-miR-3941, hsa-miR-34c-3p, hsa-miR-767-3p, hsa-miR-563, hsa-miR-548c-5p, hsa-miR-1911, hsa-miR-26b, hsa-miR-190b, hsa-miR-33a, hsa-miR-329, hsa-miR-221, hsa-miR-612, hsa-miR-3185, hsa-miR-3156-5p, hsa-miR-107, hsa-miR-664, hsa-miR-3657; (inhibitors of Ythdf2) hsa-mir-615-3p (MIRT040054), hsa-mir-106b-5p (MIRT044257), hsa-m ir-1 (MIRT023842), miR-145, hsa-miR-3607-3p, hsa-miR-200a, hsa-miR-301a, hsa-miR-519a, hsa-miR-141, hsa-miR-130b, hsa-miR-181b, hsa-miR-301b, hsa-miR-3117-3p, hsa-miR-1236, hsa-miR-181a, hsa-miR-519c-3p, hsa-miR-551b, hsa-miR-519c, hsa-miR-519b-3p, hsa-miR-19b, hsa-miR-1303, hsa-miR-608, hsa-miR-145, hsa-miR-130a, hsa-miR-181c, hsa-miR-323b-3p, hsa-miR-421, hsa-miR-515-5p, hsa-miR-3666, hsa-miR-181d, hsa-miR-146a, hsa-miR-4295, hsa-miR-454, hsa-miR-3919, hsa-miR-19a, hsa-miR-543, hsa-miR-4262 (see, e.g. Helwak et al. Cell, 153(3): 654-655, 2013; Selbach et al. Nature, 455(7209): 58-63, 2008; Yang et al. J Biol Chem., 292(9): 3614-3623, 2017); (inhibitors of Ythdf3) hsa-miR-582-3p, hsa-miR-579, hsa-miR-520c, hsa-miR-520f, hsa-miR-3152-3p, hsa-miR-106a, hsa-miR-30d, hsa-miR-30a, hsa-miR-93, hsa-miR-508-5p, hsa-miR-29a, hsa-miR-3148, hsa-miR-490-5p, hsa-miR-520b, hsa-miR-20a, hsa-miR-409-3p, hsa-miR-4255, hsa-let-7i, hsa-miR-373, hsa-Iet-7c, hsa-miR-520c-3p, hsa-miR-3920, hsa-miR-127-5p, hsa-miR-380, hsa-miR-616, hsa-miR-4277, hsa-miR-448, hsa-miR-16-2, hsa-Iet-7c, hsa-miR-340, hsa-miR-373, hsa-miR-520a-3p, hsa-miR-144, hsa-miR-1265, hsa-miR-548x, hsa-miR-362-5p, hsa-miR-33b, hsa-miR-26b, hsa-miR-17, hsa-miR-569, hsa-miR-3618, hsa-miR-576-5p, hsa-miR-922, hsa-miR-302a, hsa-miR-106b, hsa-miR-888, hsa-miR-484, hsa-Iet-7b, hsa-miR-582-5p, hsa-Iet-7f, hsa-miR-30b, hsa-miR-524-5p, hsa-miR-302d, hsa-Iet-7d, hsa-miR-513a-5p, hsa-miR-500a, hsa-miR-570, hsa-miR-548l, hsa-miR-105, hsa-miR-374c, hsa-Iet-7g hsa-miR-372, hsa-miR-3658, hsa-Iet-7a, hsa-miR-3908, hsa-miR-302b, hsa-miR-526b, hsa-miR-190, hsa-miR-181b, hsa-miR-433, hsa-miR-98, hsa-miR-3606, hsa-miR-595, hsa-miR-548 am, hsa-miR-187, hsa-miR-561, hsa-miR-181a, hsa-miR-3155, hsa-miR-655, hsa-miR-302c, hsa-miR-195, hsa-miR-26a, hsa-miR-590-3p, hsa-miR-30c, hsa-miR-502-5p, hsa-miR-495, hsa-miR-137, hsa-miR-181c, hsa-miR-520d-5p, hsa-miR-3942-5p, hsa-miR-202, hsa-miR-302c, hsa-miR-513c, hsa-miR-885-5p, hsa-miR-520a-5p, hsa-miR-583, hsa-miR-1297, hsa-miR-7-1, hsa-miR-520d-3p, hsa-miR-3155b, hsa-miR-3182, hsa-miR-519d, hsa-miR-550a, hsa-miR-7-2, hsa-miR-181d, hsa-miR-190b, hsa-miR-1912, hsa-miR-151-3p, hsa-miR-33a, hsa-miR-525-5p, hsa-miR-20b, hsa-miR-514b-5p, hsa-miR-30c, hsa-miR-4262, hsa-miR-636; (inhibitor of eIF3) hsa-m ir-92b-3p (MIRT040734), hsa-mir-16-5p (MIRT031705), hsa-mir-18a-3p (MIRT040974), hsa-mir-155-5p (MIRT020771), hsa-mir-484 (MIRT042324), hsa-let-7c-5p (MIRT051776), hsa-miR-3910, hsa-miR-148b, hsa-miR-136, hsa-miR-15a, hsa-miR-488, hsa-miR-500a, hsa-miR-1297, hsa-miR-3159, hsa-miR-374c, hsa-miR-424, hsa-miR-7-1, hsa-miR-186, hsa-miR-195, hsa-miR-15b, hsa-miR-26b, hsa-miR-505, hsa-miR-1206, hsa-miR-653, hsa-miR-1283, hsa-miR-7-2, hsa-miR-196a, hsa-miR-497, hsa-miR-33a, hsa-miR-655, hsa-miR-26a hsa-miR-16, hsa-mir-151a-3p (MIRT043600), hsa-mir-92a-3p (MIRT049064), hsa-mir-615-3p (MIRT039779), hsa-mir-877-3p (MIRT036964), hsa-mir-222-3p (MIRT046746), hsa-mir-423-3p (MIRT042468), hsa-mir-324-3p (MIRT042887), hsa-mir-124-3p (MIRT022932), hsa-miR-3140-3p, hsa-miR-124, hsa-miR-198, hsa-miR-525-5p, hsa-miR-506, hsa-miR-520a-5p, hsa-miR-196a* hsa-miR-3117-3p, hsa-mir-342-5p (MIRT038210), hsa-mir-378a-5p (MIRT043981), hsa-mir-615-3p (MIRT040086), hsa-let-7b-5p (MIRT052211), hsa-mir-455-3p (MIRT037879), hsa-miR-4267, hsa-miR-590-3p, hsa-mir-106b-5p (MIRT044355), hsa-mir-320a (MIRT044466), hsa-mir-16-5p (MIRT032018), hsa-mir-155-5p (MIRT021009), hsa-miR-4302, hsa-mir-191-5p (MIRT045793), hsa-mir-1303 (MIRT035890), hsa-mir-193b-3p (MIRT016316), hsa-mir-222-3p (MIRT046640), hsa-mir-532-3p (MIRT037924), hsa-mir-18a-3p (MIRT040929), hsa-mir-92a-3p (MIRT049001), hsa-miR-582-3p, hsa-miR-4265, hsa-miR-218-2, hsa-miR-1271, hsa-miR-340, hsa-miR-221, hsa-miR-20b, hsa-miR-508-3p, hsa-miR-141, hsa-miR-4325, hsa-miR-889, hsa-miR-29a, hsa-miR-129-3p, hsa-miR-129, hsa-miR-96, hsa-miR-3163, hsa-miR-187, hsa-miR-196a, hsa-miR-222, hsa-miR-1179, hsa-miR-182, hsa-miR-9* hsa-miR-32, hsa-miR-143, hsa-miR-4296 (see, e.g., Helwak et al. Cell, 153(3): 654-656, 2013; Selbach et al. Nature, 455(7209): 58-63, 2008; Back et al, Nature, 455(7209): 64-71, 2008; Leivonen et al. Mol Cell Proteomics, 10(7), 2011: M110.005322): (inhibitors of YTHDC1) hsa-mir-20a-3p (MIRT038967), hsa-mir-103a-3p (MIRT027037), hsa-mir-1 (MIRT023492), hsa-mir-19b-3p (MIRT031105), hsa-mir-100-5p (MIRT048400), hsa-mir-93-5p (MIRT027989), hsa-mir-16-5p (MIRT031379), hsa-let-7b-5p (MIRT052150), hsa-miR-520f, hsa-miR-300, hsa-miR-15a, hsa-miR-200a, hsa-miR-605, hsa-miR-30d, hsa-miR-30a, hsa-miR-3613-3p, hsa-miR-509-3-5p, hsa-miR-34c-5p, hsa-miR-324-3p, hsa-miR-1248, hsa-miR-152, hsa-miR-548t, hsa-miR-4310, hsa-miR-145, hsa-miR-516a-3p, hsa-miR-16, hsa-miR-3668, hsa-miR-4277, hsa-miR-448, hsa-miR-16-2, hsa-miR-148b, hsa-miR-509-5p, hsa-miR-103a, hsa-miR-1265, hsa-miR-2115, hsa-miR-548c-3p, hsa-miR-148a, hsa-miR-548p, hsa-miR-513a-3p, hsa-miR-497, hsa-miR-3647-3p, hsa-miR-382, hsa-miR-30b, hsa-miR-543, hsa-let-7f-2, hsa-miR-1269, hsa-miR-3164, hsa-miR-503, hsa-miR-500a, hsa-miR-449a, hsa-miR-141, hsa-miR-424, hsa-miR-3908, hsa-miR-889, hsa-miR-2116, hsa-miR-330-3p, hsa-miR-15b, hsa-miR-181b, hsa-miR-187, hsa-miR-1237, hsa-miR-449b, hsa-miR-101, hsa-miR-381, hsa-miR-618, hsa-miR-222, hsa-miR-181a, hsa-miR-432, hsa-miR-96, hsa-miR-19b, hsa-miR-195, hsa-miR-548n, hsa-miR-485-5p, hsa-miR-217, hsa-miR-30c, hsa-miR-495, hsa-miR-137, hsa-miR-1288, hsa-miR-181c, hsa-miR-3942-5p, hsa-miR-548v, hsa-miR-487a, hsa-miR-221, hsa-miR-891b, hsa-miR-205, hsa-miR-195, hsa-miR-4271, hsa-miR-3611, hsa-miR-516b, hsa-miR-181d, hsa-miR-154, hsa-miR-646, hsa-miR-153, hsa-miR-34a, hsa-miR-19a, hsa-miR-107, hsa-miR-30c, hsa-miR-4262, and a combination thereof (see, e.g. Helwak et al. Cell, 153(3): 654-655, 2013; Hafner et al. Cell, 141(1): 129-141, 2010; Kishore et al, Nat Methods, 8(7): 559-64, 2011; Memczak et al. Nature, 495(7441): 333-8, 2013; Selbach et al. Nature, 455(7209): 58-63, 2008; Chi et al. Nature. 460(7254): 479-86, 2009).

### 2. Small Molecule YTHDF2 Inhibitors

In some aspects, a YTHDF2 inhibitor is a small molecule capable of binding to YTHDF2 protein. In some aspects, a YTHDF2 inhibitor is a small molecule that inhibits interaction of YTHDF2 protein with m⁶A-containing mRNA. The YTHDF2 small molecule inhibitor may be any small molecule inhibitor known in the art. The small molecule inhibitor may include any small molecule known to inhibit the interaction of YTHDF2 protein with m⁶A-containing mRNA.

The term “small molecule” includes any chemical or other moiety, other than polypeptides and nucleic acids, that can act to affect biological processes, particularly to modulate members of the m6A mRNA modification pathway. Small molecules can include any number of therapeutic agents presently known and used, or that can be synthesized in a library of such molecules for the purpose of screening for biological function(s). Small molecules are distinguished from macromolecules by size. The small molecules of the present invention usually have a molecular weight less than about 5,000 daltons (Da), preferably less than about 2,500 Da, more preferably less than 1,000 Da, most preferably less than about 500 Da.

### 3. Radiotherapy

In some aspects, the disclosed methods comprise administering radiotherapy, such as ionizing radiation, as a cancer therapy to a subject or patient. As used herein, “ionizing radiation” means radiation comprising particles or photons that have sufficient energy or can produce sufficient energy via nuclear interactions to produce ionization (gain or loss of electrons). A preferred non-limiting example of ionizing radiation is an x-radiation. Means for delivering x-radiation to a target tissue or cell are well known in the art.

In some aspects, the radiotherapy can comprise external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor. In some aspects, the radiotherapy is administered to a metastatic tumor.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 0.5, 1, 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of radiotherapy administered to a subject may be presented as a total dose of radiotherapy, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of radiation is at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 Gy (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein). In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### C. Cancer Immunotherapy

In some aspects, the disclosed methods comprise administering a cancer immunotherapy as a cancer therapy to a subject or patient. Cancer immunotherapy is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Aspects of the disclosure may include administration of immune checkpoint inhibitors or immune blockade therapies, examples of which are further described below. As disclosed herein, “checkpoint inhibitor therapy” (also “immune checkpoint blockade therapy”, “immune checkpoint therapy”, “ICT.” “checkpoint blockade immunotherapy.” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4. B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. pat. No. 8,17,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some aspects, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-33, MGD013, BI754111, AVA-17, or GSK2831781. The anti-LAG3 antibodies disclosed in: U.S. Pat. No. 9,505,839 (BMS-986016, also known as relatlimab); US 10,711,60 (IMP-701, also known as LAG525); US 9,244,59 (IMP731, also known as H5L7BW); US 10,344,89 (25F7, also known as LAG3.1); WO 2016/028672 (MK-4280, also known as 28G-10); WO 2017/019894 (BAP050); Burova E., et al., J. ImmunoTherapy Cancer, 2016; 4 (Supp. 1): P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-LAG3 antibody. Accordingly, in one aspect, the inhibitor comprises the CDR1, CDR2, and CDR3 domains of the VH region of an anti-LAG3 antibody, and the CDR1, CDR2 and CDR3 domains of the VL region of an anti-LAG3 antibody. In another aspect, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some aspects, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-22 (also known as Cobolimab), and LY3321367 can be used in the methods disclosed herein. These and other anti-TIM-3 antibodies useful in the claimed disclosure can be found in, for example: US 9,605,70, U.S. Pat. No. 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some aspects, the inhibitor comprises the heavy and light chain CDRs or VRs of an anti-TIM-3 antibody. Accordingly, in one aspect, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another aspect, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein. For example, the patient may be one that has been determined to be sensitive to an immune checkpoint inhibitor therapy based on a determination that the patient has or previously had pancreatitis.

### D. Oncolytic Virus

In some aspects, the cancer therapy to be administered to the subject comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### E. Polysaccharides

In some aspects, the cancer therapy to be administered to the subject comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### F. Neoantigens

In some aspects, the cancer therapy to be administered to the subject comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### G. Chemotherapies

In some aspects, the cancer therapy to be administered to the subject comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain aspects. In some aspects, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### H. Surgery

In some aspects, the cancer therapy to be administered to the subject comprises one or more surgeries. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### II. Cancer Treatment

Aspects of the present disclosure are directed to methods comprising treatment of a subject suffering from, or suspected of having, cancer. In some aspects, the cancer is glioma, sarcoma, liver, lung, colon, or melanoma. In certain aspects, the disclosed methods comprise treating a subject who currently has or has previously had resistance to radiotherapy and/or immunotherapy. A subject may be identified as having resistance to radiotherapy and/or immunotherapy using tests and diagnostic methods known in the art.

As used herein, “treat.” “treating.” or “treatment” or equivalent terminology refer to both therapeutic treatment and prophylactic or preventative measures, wherein the object is to prevent or slow down (lessen) an undesired physiological change or disorder, such as the growth, development, or spread of one or more symptoms or manifestation of a disease or condition. As an example, the disease or condition may be cancer, and the one or more symptoms may be, for example, symptoms associated with the cancer. For purposes of this disclosure, beneficial or desired clinical results include, but are not limited to, alleviation or amelioration of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” does not necessarily indicate complete eradication or cure of the disease or condition, or associated symptoms thereof. Those in need of treatment include those already with the condition or disorder as well as those prone to have the condition or disorder or those in which the condition or disorder is to be prevented. The results of treatment can be determined by methods known in the art, such as determination of reduction of, e.g., tumor burden, determination of restoration of function, or other methods known in the art.

As used herein, “prevent,” and similar words such as “prevented,” “preventing.” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition. Prevention may be considered complete when onset of a disease, disorder, or condition has been delayed for a predefined period of time.

In some aspects, the disclosed methods comprise treating a subject suffering from a cancer with a combination of a YTHDF2 inhibitor and a cancer therapy. As disclosed herein, cancers associated with resistance to radiotherapy and/or immunotherapy are surprisingly and unexpectedly sensitive to treatment with a combination of a YTHDF2 inhibitor and a cancer therapy, e.g., radiotherapy and/or an immunotherapy. Further, administering a YTHDF2 inhibitor and a cancer therapy was surprisingly found to decrease immunosuppression of a systemic response to an radiotherapy and/or an immunotherapy treatment.

Accordingly, in some aspects, disclosed is a method for treating a subject suffering from cancer with a YTHDF2 inhibitor and a cancer therapy. In some aspects, administration of the YTHDF2 inhibitor decreases suppression of an immune response to the cancer therapy. Also disclosed herein are methods for increasing the efficacy of a cancer therapy in a subject by administering a YTHDF2 inhibitor and the cancer therapy.

In further aspects, treating a subject suffering from cancer comprises decreasing myeloid-derived suppressor cell trafficking and function in the subject by administering a YTHDF2 inhibitor and a cancer therapy. Also disclosed, in some aspects, is a method of decreasing myeloid-derived suppressor cell trafficking and function in a subject by administering a YTHDF2 inhibitor. In some aspects, administration of the YTHDF2 inhibitor decreases myeloid-derived suppressor cell trafficking and function in the subject. In some aspects, decreasing myeloid-derived suppressor cell trafficking and function in the subject helps to treat or prevent cancer. In some aspects, decreasing myeloid-derived suppressor cell trafficking and function in the subject helps to increase the efficacy of a cancer therapy in a subject.

In further aspects, treating a subject suffering from cancer comprises inhibiting NF-κB signaling in the subject by administering a YTHDF2 inhibitor and a cancer therapy. In some aspects, administration of the YTHDF2 inhibitor inhibits NF-κB signaling in myeloid-derived suppressor cells in the subject. Also disclosed, in some aspects, is a method of inhibiting NF-κB signaling in myeloid-derived suppressor cells in a subject by administering a YTHDF2 inhibitor. In some aspects, administration of the YTHDF2 inhibitor inhibits NF-κB signaling in myeloid-derived suppressor cells in the subject. In some aspects, inhibiting NF-κB signaling in myeloid-derived suppressor cells in the subject helps to treat or prevent cancer. In some aspects, inhibiting NF-κB signaling in myeloid-derived suppressor cells in the helps to increase the efficacy of a cancer therapy in a subject.

In some aspects, the cancer is a cancer characterized as being resistant to radiotherapy and/or an immunotherapy treatment.

In some aspects, the radiotherapy comprises external radiotherapy, internal radiotherapy, radioimmunotherapy, or intraoperative radiation therapy (IORT). In some aspects, the external radiotherapy comprises three-dimensional conformal radiation therapy (3D-CRT), intensity modulated radiation therapy (IMRT), proton beam therapy, image-guided radiation therapy (IGRT), or stereotactic radiation therapy. In some aspects, the internal radiotherapy comprises interstitial brachytherapy, intracavitary brachytherapy, or intraluminal radiation therapy. In some aspects, the radiotherapy is administered to a primary tumor. In some aspects, the radiotherapy is administered to a metastatic tumor.

In some aspects, the checkpoint inhibitor therapy comprises a cytotoxic T-lymphocyte-associated protein 4 (CTLA4) inhibitor, a programmed cell death protein 1 (PD1) inhibitor, a programmed death-ligand 1 (PDL-1) inhibitor, a lymphocyte activation gene-3 (LAG3) inhibitor, or a T cell immunoglobulin and mucin domain 3 (TIM-3) inhibitor. In some aspects, the checkpoint inhibitor therapy comprises a PD1 inhibitor. In some aspects, the PD1 inhibitor is an anti-PD1 antibody. In some aspects, the checkpoint inhibitor therapy comprises a PDL-1 inhibitor. In some aspects, the PDL-1 inhibitor is an anti-PDL-1 antibody.

In some aspects, the disclosed methods comprise identifying one or more subjects as being candidates for treatment with a combination of a YTHDF2 inhibitor and a cancer therapy, e.g., radiotherapy and/or an immunotherapy, based on current or former resistance to treatment with a cancer therapy, e.g., radiotherapy and/or an immunotherapy. For example, in some aspects, disclosed is a method comprising identifying a subject having cancer as being a candidate for treatment with a combination of a YTHDF2 inhibitor and a cancer therapy, e.g., radiotherapy and/or an immunotherapy, by determining that the subject currently has or previously had resistance to treatment with an radiotherapy and/or an immunotherapy. In some aspects, the disclosed methods comprise determining an optimal cancer treatment for a subject with resistance to treatment with radiotherapy and/or an immunotherapy. In some aspects, a subject is given multiple types of cancer therapy, for example radiotherapy, a cancer immunotherapy, and/or a chemotherapy.

Also disclosed in some aspects are pharmaceutical compositions comprising a YTHDF2 inhibitor. In some aspects, the pharmaceutical compositions can further comprise one or more additional therapeutics, for example, an immunotherapy, e.g., a checkpoint inhibitor.

### III. Administration of Therapeutic Compositions

The therapy provided herein may comprise administration of a combination of therapeutic agents, such as a first cancer therapy (e.g., a radiotherapy or an immunotherapy, for example, a checkpoint inhibitor therapy) and a YTHDF2 inhibitor. The therapies may be administered in any suitable manner known in the art. For example, the YTHDF2 inhibitor and the cancer therapy may be administered sequentially (at different times) or concurrently (at the same time or approximately the same time; also “simultaneously” or “substantially simultaneously”). In some aspects, the YTHDF2 inhibitor and the cancer therapy are administered in separate compositions. In some aspects, the YTHDF2 inhibitor and the cancer therapy are in the same composition.

In some aspects, the YTHDF2 inhibitor and the cancer therapy are administered substantially simultaneously. In some aspects, the YTHDF2 inhibitor and the cancer therapy are administered sequentially. In some aspects, the YTHDF2 inhibitor is administered before administering the cancer therapy. In some aspects, the YTHDF2 inhibitor is administered after administering the cancer therapy. In some aspects, a first dose of the YTHDF2 inhibitor is administered before administering the cancer therapy and further dose(s) of the YTHDF2 inhibitor are administered after administering the cancer therapy.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intratumorally, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the YTHDF2 inhibitor is administered. In some aspects, multiple doses of the YTHDF2 inhibitor are administered. In some aspects, the YTHDF2 inhibitor is administered at a dose of between 1 mg/kg and 5000 mg/kg. In some aspects, the YTHDF2 inhibitor is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

In some aspects, a single dose of the immunotherapy is administered. In some aspects, multiple doses of the immunotherapy are administered. In some aspects, the immunotherapy is administered at a dose of between 1 mg/kg and 100 mg/kg. In some aspects, the immunotherapy is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/kg.

In some aspects, the radiotherapy administered to the subject provides irradiation in a dose range of 0.5 Gy to 60 Gy. In some aspects, the radiotherapy administered to the subject provides irradiation at a dose of at least, at most, or about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 Gy. In some aspects, the radiotherapy is administered in a single dose. In some aspects, the radiotherapy is administered in a fractionated dose over a period of time of not more than one week. In some aspects, the radiotherapy is delivered in a fractionated dose over a period of time of not more than three days.

The quantity to be administered, both according to number of treatments and unit dose, depends on the treatment effect desired. The term “therapeutic benefit” or “therapeutically effective” as used throughout this application refers to anything that promotes or enhances the well-being of the subject with respect to the medical treatment of cancer. This includes, but is not limited to, a reduction in the frequency or severity of the signs or symptoms of a disease. For example, treatment of cancer may include but is not limited to total or partial remission of the cancer. Treatment of cancer may also refer to prolonging survival of a subject with a cancer. The term “therapeutically effective amount” refers to an amount sufficient to produce a desired therapeutic result, for example an amount of a YTHDF2 inhibitor and/or a cancer therapy or a composition comprising such a YTHDF2 inhibitor and/or a cancer therapy sufficient to improve at least one symptom of a medical condition in a subject to whom the YTHDF2 inhibitor and/or a cancer therapy or composition thereof are administered.

In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 M; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. General Pharmaceutical Compositions

In some embodiments, pharmaceutical compositions are administered to a subject. Different aspects may involve administering an effective amount of a composition to a subject. In some embodiments, an antibody or antigen binding fragment capable of binding to an antigen may be administered to the subject to protect against or treat a condition (e.g., cancer). Alternatively, an expression vector encoding one or more such antibodies or polypeptides or peptides and/or one or more viral proteins may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Diagnostic and Prognostic Methods

Certain aspects relate to diagnosing or prognosing an individual. The methods may be used to determine the effectiveness of one or more therapies in treating cancer in the individual. The therapies may comprise radiotherapy and/or an immunotherapy. In certain aspects, the methods relate to measuring a YTHDF2 gene product in a sample. The sample may be a blood sample, which can be further prepared, purified, modified, and/or enhanced. In some aspects, the sample is prepared to enrich the sample for certain cell types. In some aspects, the sample is prepared to enrich the sample for peripheral blood mononuclear cells (PBMCs). In some aspects, the sample is prepared to enrich the sample for myeloid-derived suppressor cells (MDSCs). The sample may comprise PBMCs. The sample may comprise MDSCs.

### A. Sample Preparation

In certain aspects, methods involve obtaining a sample from a subject. The subject may have, or be suspected of having cancer. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### B. Protein Assays

A variety of techniques can be employed to measure expression levels of polypeptides and proteins, including YTHDF2, in a biological sample to determine biomarker expression levels. Examples of such formats include, but are not limited to, enzyme immunoassay (EIA), radioimmunoassay (RIA), Western blot analysis, immunohistochemistry, and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, flow cytometry, or immunofluorescence techniques to detect biomarker expression. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horseradish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 125I, 3H, and 131I. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73:147-166(1981).

In some aspects, a detection label is indirectly conjugated with an antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). In some aspects, the antibody need not be labeled, and the presence thereof can be detected using a labeled antibody, which binds to the antibody.

### C. Monitoring

In certain aspects, the biomarker-based method may be combined with one or more other cancer diagnosis or screening tests at increased frequency if the patient is determined to be at high risk for recurrence or have a poor prognosis based on the biomarker as described above.

In some aspects, the methods of the disclosure further include one or more monitoring tests. The monitoring protocol may include any methods known in the art. In particular,

the monitoring include obtaining a sample and testing the sample for diagnosis. For example, the monitoring may include endoscopy, biopsy, laparoscopy, colonoscopy, blood test, genetic testing, endoscopic ultrasound, X-ray, barium enema x-ray, chest x-ray, barium swallow, a CT scan, a MRI, a PET scan, or HER2 testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

### VI. Kits

Certain aspects of the present disclosure also concern kits containing compositions of the disclosure or compositions to implement methods of the disclosure. Any of the compositions described herein may be comprised in a kit. In a non-limiting example, a YTHDF2 inhibitor and a cancer therapy may be comprised in a kit. Such a kit may or may not have one or more reagents for manipulation of cells. Such reagents include small molecules, proteins, nucleic acids, antibodies, buffers, primers, nucleotides, salts, and/or a combination thereof, for example.

In particular aspects, the kit comprises a YTHDF2 inhibitor of the disclosure and also another cancer therapy. In some cases, the kit, in addition to the YTHDF2 inhibitor and cancer therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### EXAMPLES

The following examples are included to demonstrate aspects of the disclosure. It should be appreciated by those of skill in the art that the techniques disclosed in the examples which follow represent techniques discovered by the inventor to function well in the practice of the disclosure. However, those of skill in the art should, in light of the present disclosure, appreciate that many changes can be made in the specific aspects which are disclosed and still obtain a like or similar result without departing from the spirit and scope of the disclosure.

### Example 1—Local Tumor Irradiation Increases Tumor Associated Myeloid Cells Expressing YTHDF2

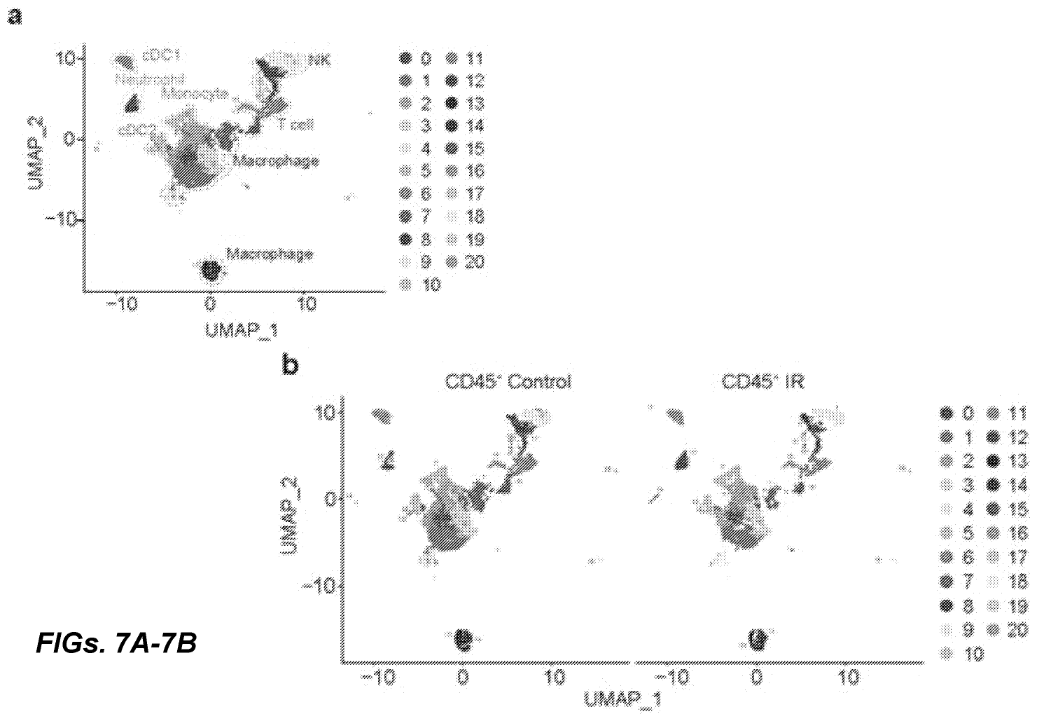

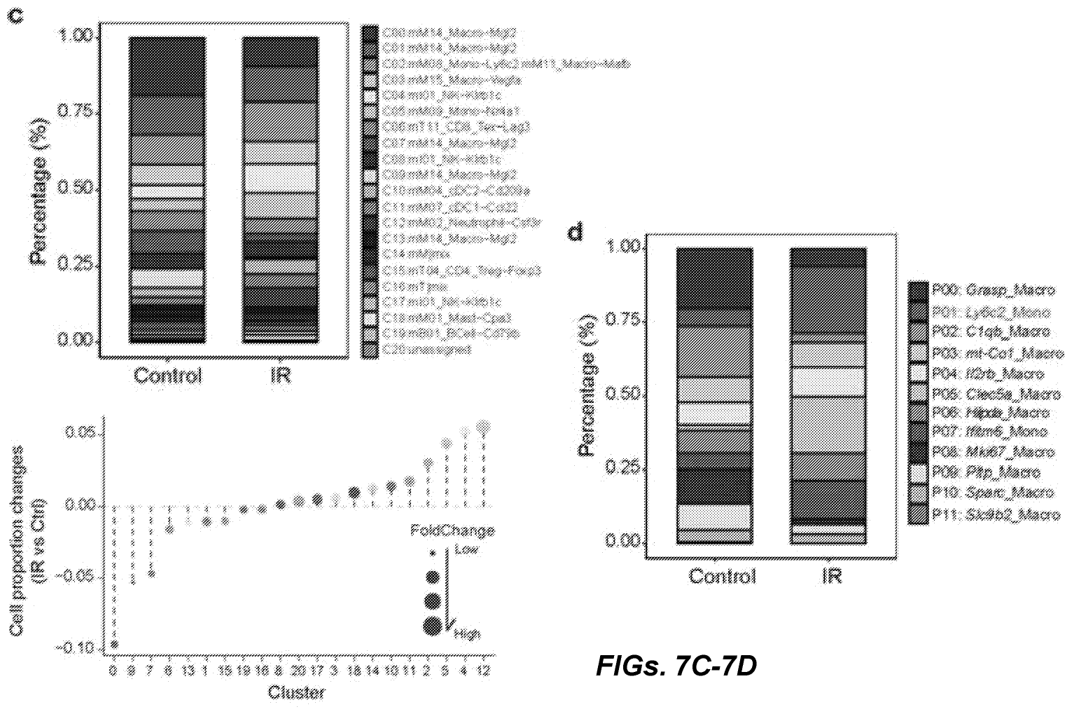

To investigate the cellular and molecular contexture of the tumor immune microenvironment (TIME) following high dose radiation used in ablative radiotherapy, the inventors characterized CD45+ immune cells isolated from irradiated (4 days after 20 Gy-IR) and non-irradiated MC38 tumors by high-throughput single-cell RNA sequencing (scRNA-seq). The inventors identified five major cell lineages including T cells, natural killer (NK) cells, dendritic cells (DCs), monocytes, and macrophages, based on gene expression signatures (FIG. 7A). The inventors then characterized the changes of these cell subtypes in irradiated tumors compared with non-irradiated tumors. The proportion of T cells was slightly decreased, while an NK subset (Klrb1c_NK, C04), two DC subsets (Ccl22_cDC1, C11 and Cd209a_cDC2, C10); distinct subsets of macrophages and monocytes (C03 and C05), and neutrophil subsets (C12) were increased post-IR (FIGS. 7B-C). Of note, C03 cells showed upregulated Vegfa expression and C05 cells showed upregulated Nr4a1 expression, suggesting their tendency towards the M2 phenotype. Thus, IR markedly changes the TIME in ways that induce both immune enhancing (NK and DCs) and immune suppressive responses (macrophages and neutrophils).

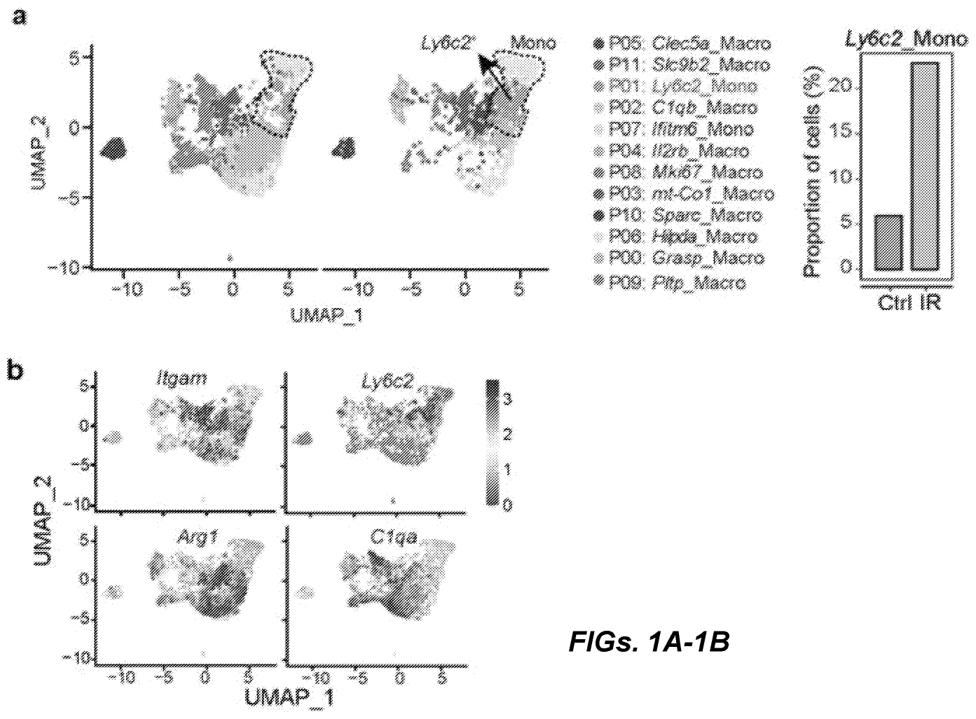

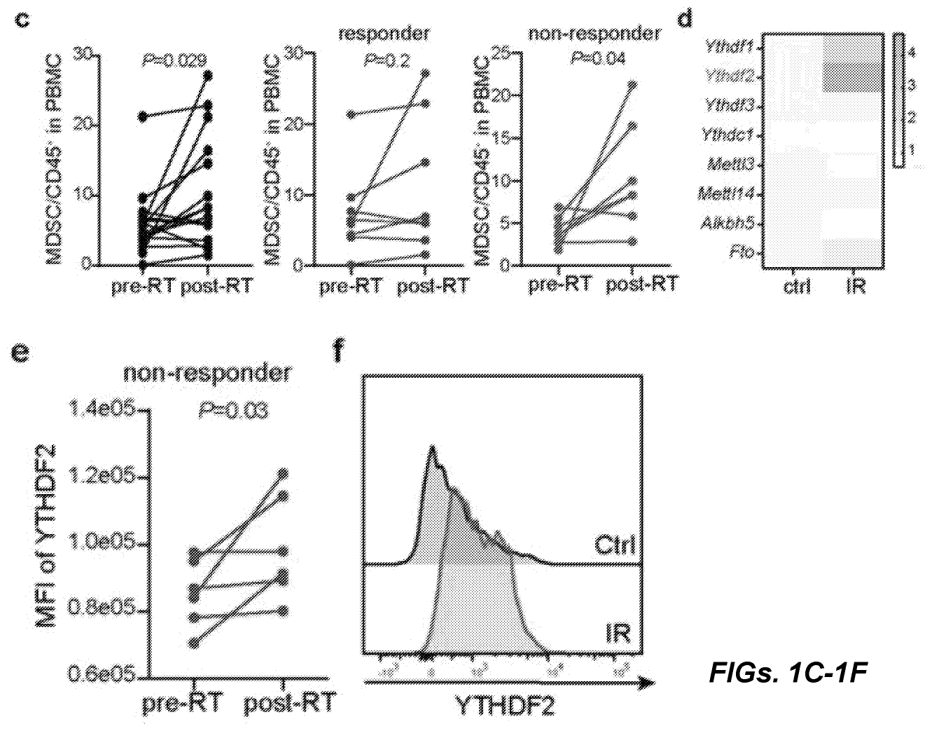

Focusing on the myeloid compartment, the inventors identified a monocytic MDSC cell subset (Ly6c2_Mono) in mice that dramatically increased in tumors post-IR (FIG. 1A, FIG. 7D), characterized by low expression of C1qa, a macrophage marker, and high expression of Ly6c2, CD11b and Arg1 (FIG. 1B). This observation is consistent with previous work identifying Ly6C+ monocytes as MDSCs with superior T cell suppressive function¹¹. In an effort to translate this finding to humans, the inventors evaluated the levels of MDSCs in PBMCs from cancer patients enrolled in a clinical trial; patients were treated with radiotherapy followed by pembrolizumab (anti-PD-1)¹⁸. MDSCs were significantly increased post-RT compared with matched pre-RT levels (P=0.29) in PBMCs. Notably, this increase was pronounced in patients who progressed at distal sites (outside of the radiation treatment fields) (P=0.04), but was not significantly changed in patients who did not progress at distal sites (P=0.2) (FIG. 1C). These data indicate that radiation-induced MDSCs may be associated with worse clinical outcome in patients who receive local radiotherapy.

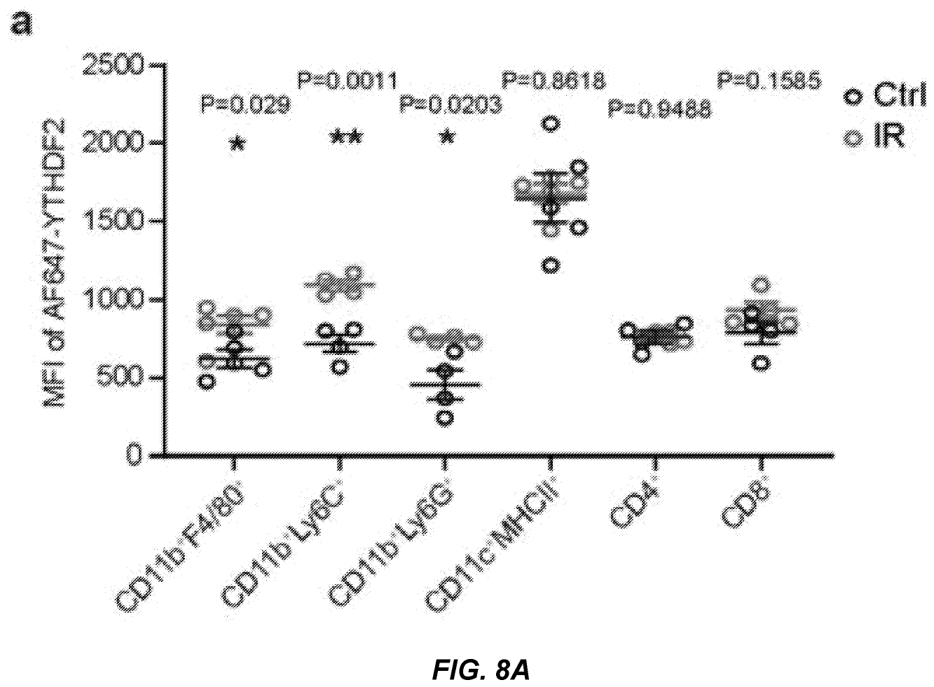

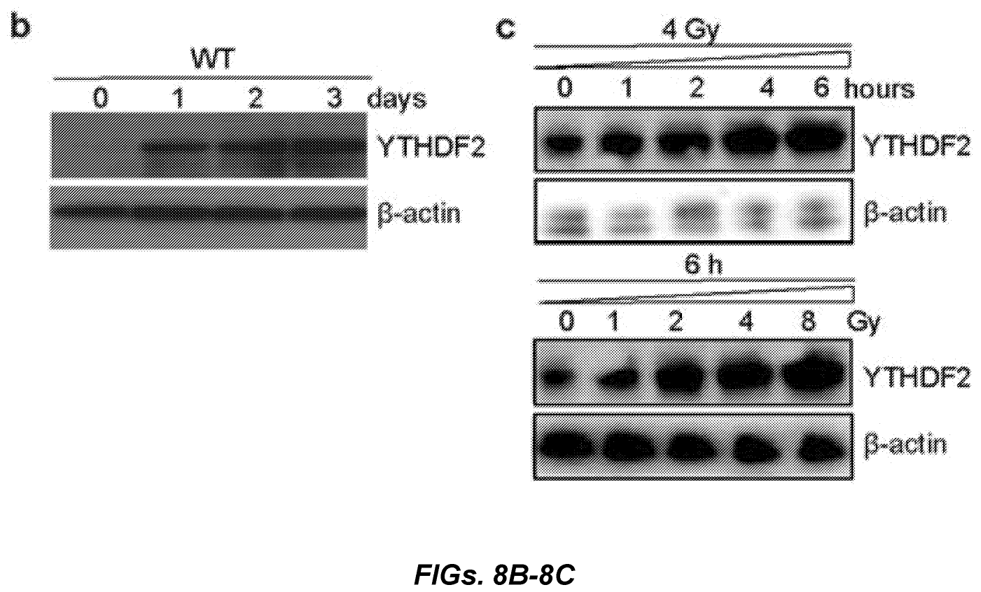

In addition to increased numbers of immunosuppressive MDSCs post-RT, the inventors also investigate the impact of IR on epitranscriptomic modifications driven by RNA m6A methylation, which the inventors have recently shown also modulates host antitumor immunity¹⁹. The inventors observed that the expression of YTHDF2, a protein regulator of m6A-methylated mRNA20, was dramatically elevated in MDSCs of irradiated versus untreated tumors (FIG. 1D). In the PBMCs of patients with distal tumor progression, the YTHDF2 level in myeloid cells was significantly increased post-RT compared with matched pre-RT samples/values (P=0.03. FIG. 1E). Consistently. YTHDF2 protein level was markedly induced in MC38 tumor-infiltrating MDSCs after IR treatment (FIG. 1F), but not in other infiltrating immune cells, including DCs and T cells (FIG. 8A). The inventors next interrogated the temporal response of YTHDF2 following IR and observed that YTHDF2 was markedly elevated in a time-dependent manner (FIG. 8B). IR also elicited a direct effect on YTHDF2 expression, as evidenced by upregulated YTHDF2 in bone-marrow derived CD11b+Ly6C+ cells treated with IR in a time- and dose-dependent manner (FIG. 8C). These results demonstrate that IR induces YTHDF2 expression in MDSCs in both clinical and preclinical settings.

### Example 2—Ythdf2 Deficiency in Myeloid Cells Improves Response to Radiotherapy

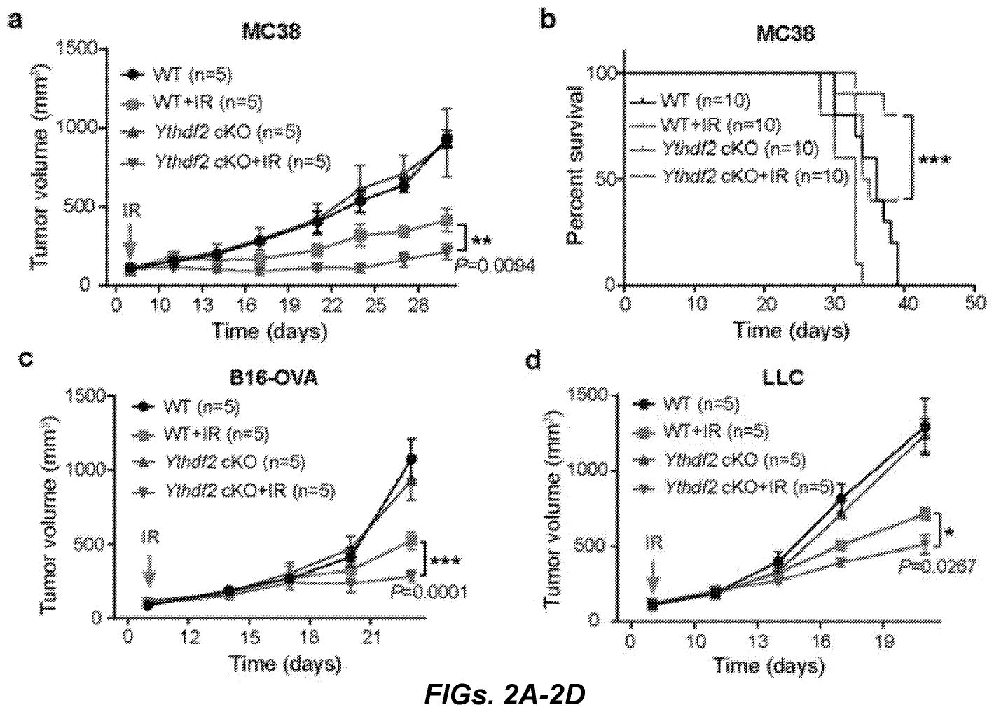

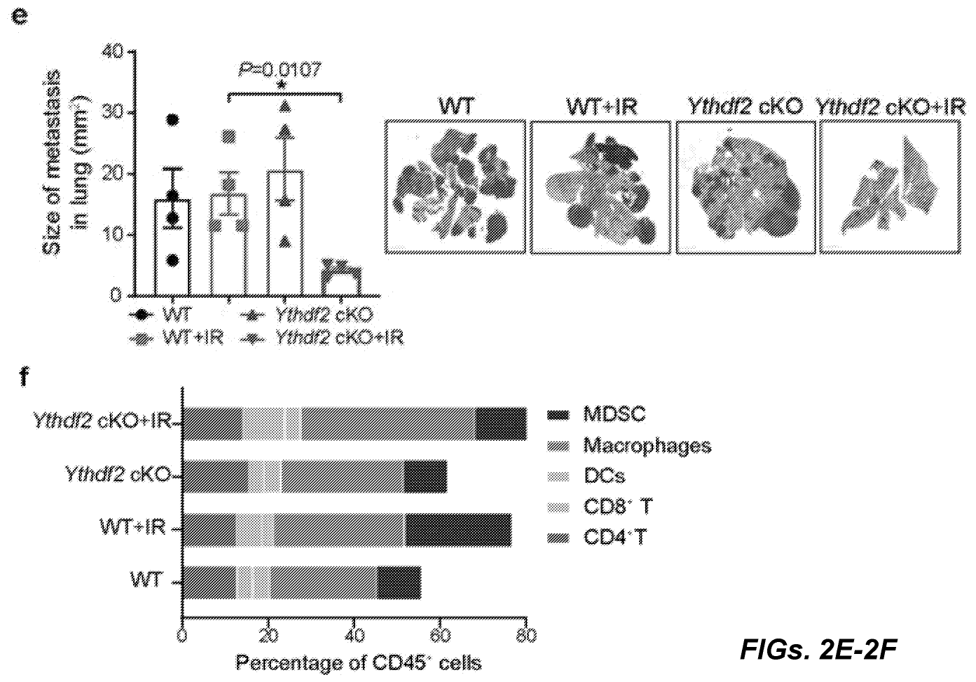

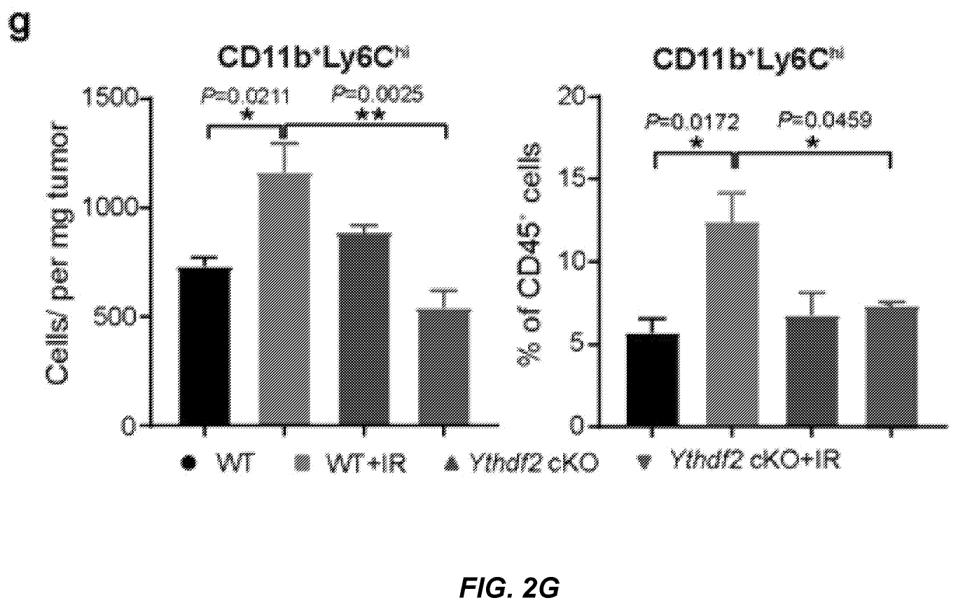

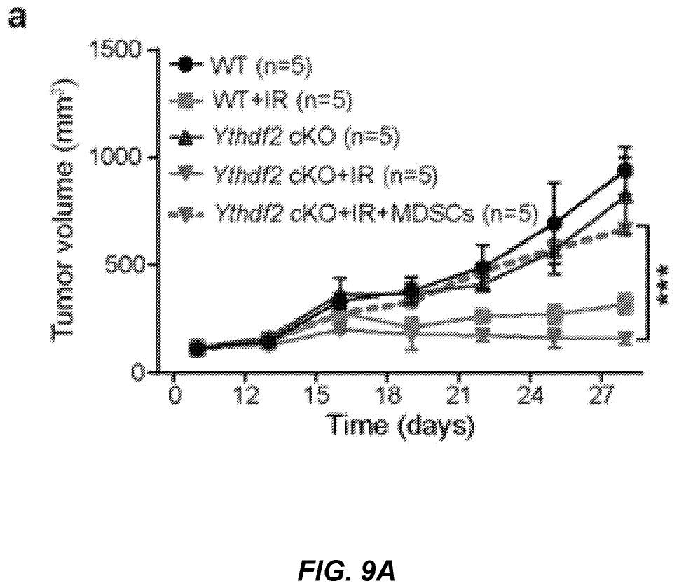

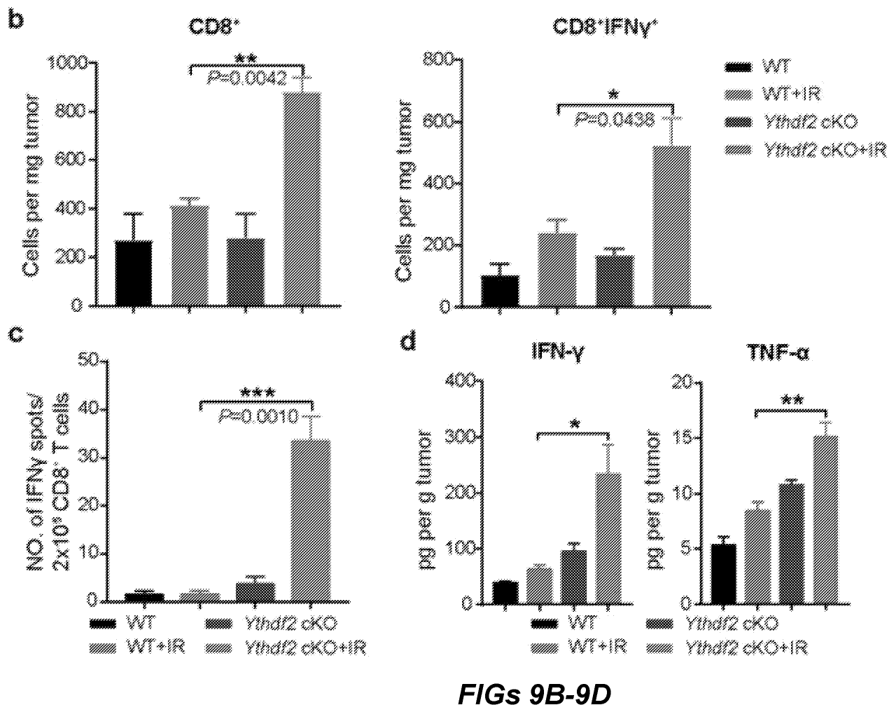

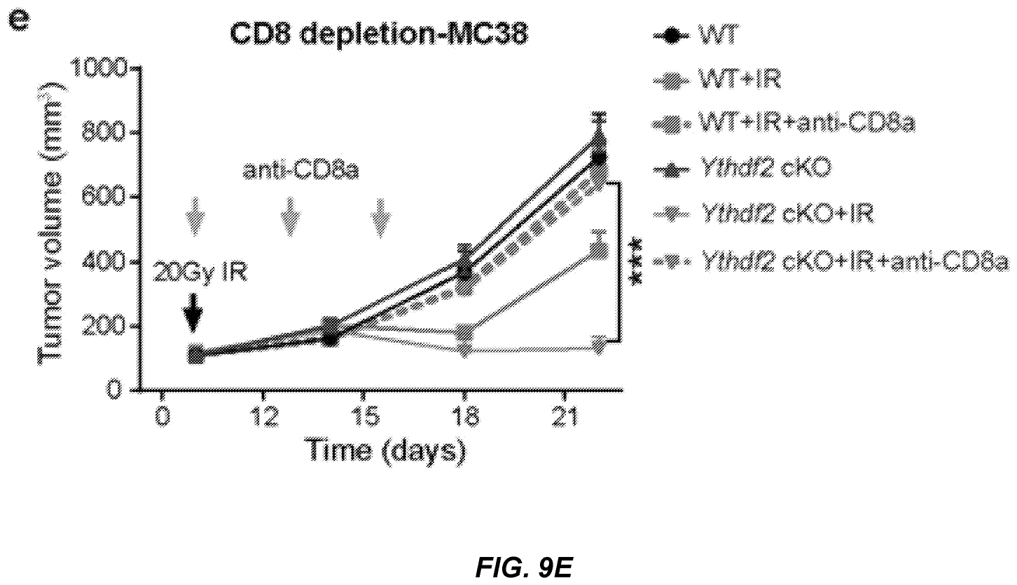

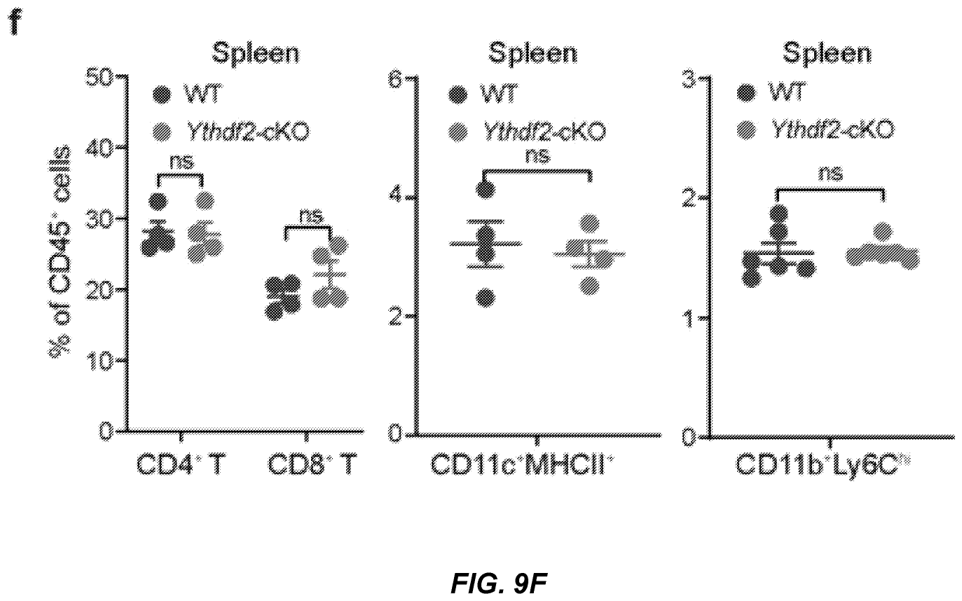

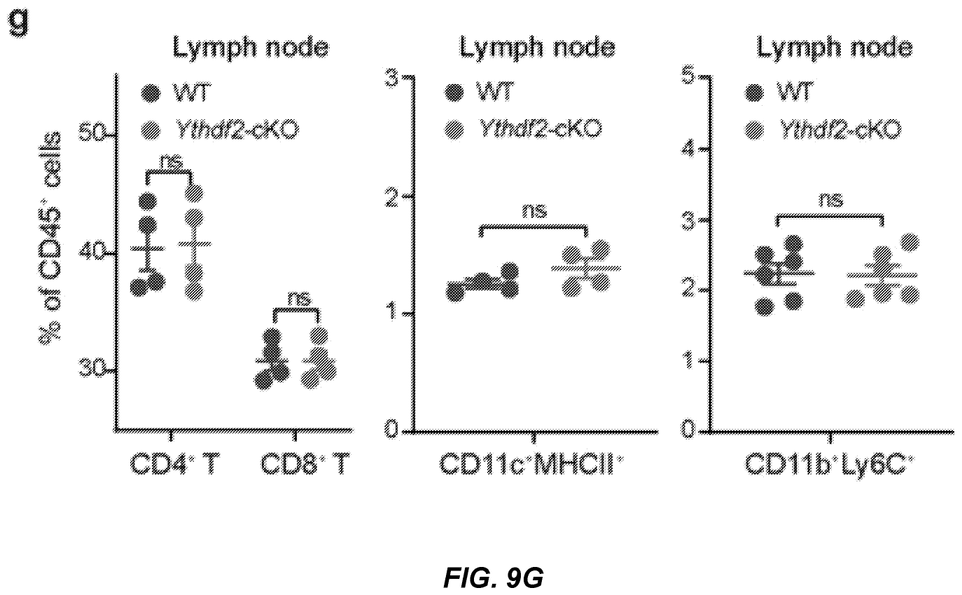

The inventors reasoned that increased YTHDF2 in myeloid cells could alter the response to radiotherapy. To test this, the inventors employed Lyz^{Cre+}Ythdf2^{fl/fl} conditional knockout mice (hereafter Ythdf2-cKO) and Ythdf2^{fl/fl} (hereafter WT) in the C57BL/6J genetic background for tumor growth experiments. In the syngeneic murine colon carcinoma (MC38) model, primary tumor growth in WT and Ythdf2-cKO mice were similar (FIG. 2A). By contrast, local irradiation treatment resulted in a pronounced inhibition of tumor growth in Ythdf2-cKO mice compared with WT mice, assessed by both tumor volume and animal survival (FIGS. 2A-B). The inventors also irradiated melanoma and Lewis lung carcinoma (LLC) tumors in both WT and Ythdf2-cKO mice, and observed a similar phenotype (FIGS. 2C-D). Intriguingly, in the LLC spontaneous lung metastasis model, a reduced metastatic burden was observed in lungs of Ythdf2-cKO mice that received IR compared with WT mice that received IR (FIG. 2E). Taken together, these data indicate that deleting Ythdf2 in myeloid cells enhanced the efficacy of radiotherapy through an increase in both local and distal metastasis control. To assess whether distinct myeloid cell subsets are implicated in tumor progression and antitumor immunity following IR, the inventors first characterized the effects of Ythdf2 deletion in the TIME after IR by profiling the tumor-infiltrating immune cells in MC38 tumors using flow cytometry. MDSCs and CD8⁺ T cells exhibited significant changes (FIG. 2F). In WT mice, both the absolute number and percentage of CD11b⁺Ly6C^{hi} cells (monocytic MDSCs) increased in irradiated tumors compared with controls. In Ythdf2-cKO mice, the level of MDSCs did not increase in irradiated tumors and remained similar to the level in non-irradiated tumors from WT mice (FIG. 2G). The level of MDSCs in irradiated tumors from Ythdf2-cKO mice was significantly decreased both in absolute number (P=0.0025) and percentage (P=0.0459; FIG. 2G) compared with irradiated tumors in WT mice. Furthermore, the inventors intratumorally injected CD45.1 wild-type MDSCs into MC38 tumor bearing Ythdf2-cKO mice treated with IR and found that the enhanced antitumor effect of IR in Ythdf2-cKO mice was abolished (FIG. 9A). The results demonstrate that Ythdf2 deletion in myeloid cells led to a reduction of tumor-infiltrating MDSCs. To examine whether specific deletion of myeloid Ythdf2 results in improved immune function of T cells. The inventors first measured the numbers of T cells and observed that both total CD8⁺ T cells and cytotoxic CD8⁺ T cells (IFNγ⁺CD8⁺) were significantly increased in irradiated tumors in Ythdf2-cKO mice compared with those of irradiated tumors from WT mice (FIG. 9B). ELISPOT assays measuring the IFN-γ secreting capacity of CD8⁺ T cells sorted from the irradiated tumors in Ythdf2-cKO mice consistently showed a significant increase of IFN-γ production (FIG. 9C). The inventors also found increased levels of both IFN-γ and tumor necrosis factor (TNF)-α (FIG. 9D), representing enhanced cytotoxic function. The antibody-mediated depletion of CD8⁺ T cells completely abrogated the antitumor efficacy of IR in Ythdf2-cKO mice (FIG. 9E). Considering that conditional knockout of Ythdf2 did not affect the development of T cells in naïve mice (FIGS. 9F-G), the inventors' findings indicate that the CD8⁺ T cells are essential for enhanced IR-induced tumor control likely due to decreased MDSCs in Ythdf2-cKO mice.

### Example 3—IR Reshapes the Composition of MDSC Populations in Blood and Tumors

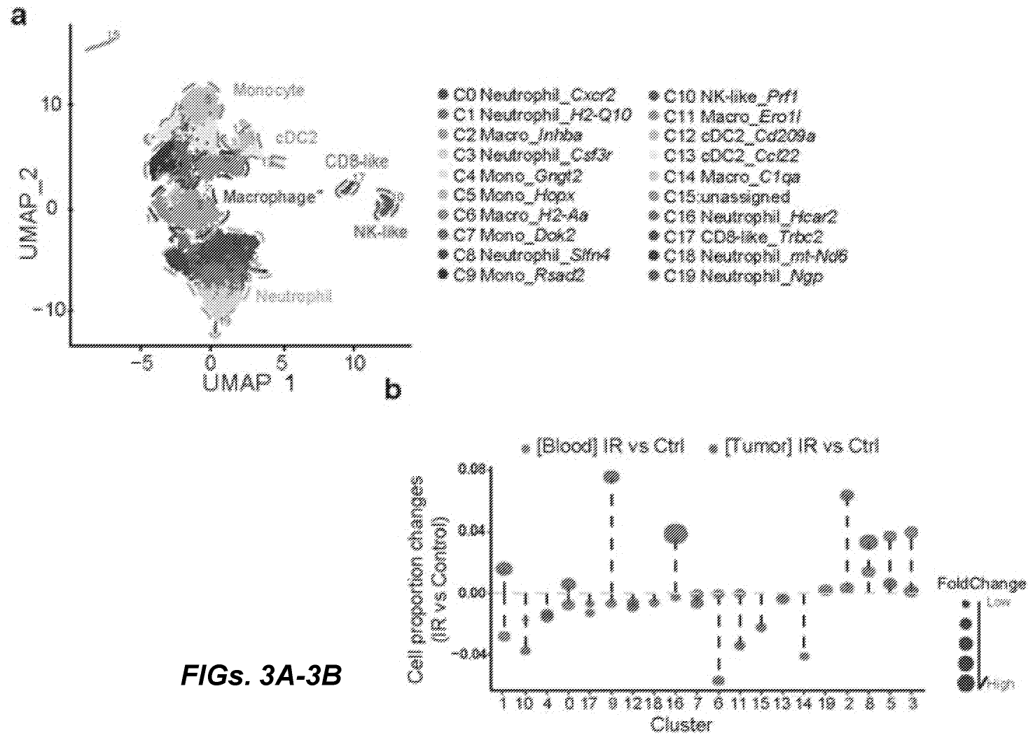

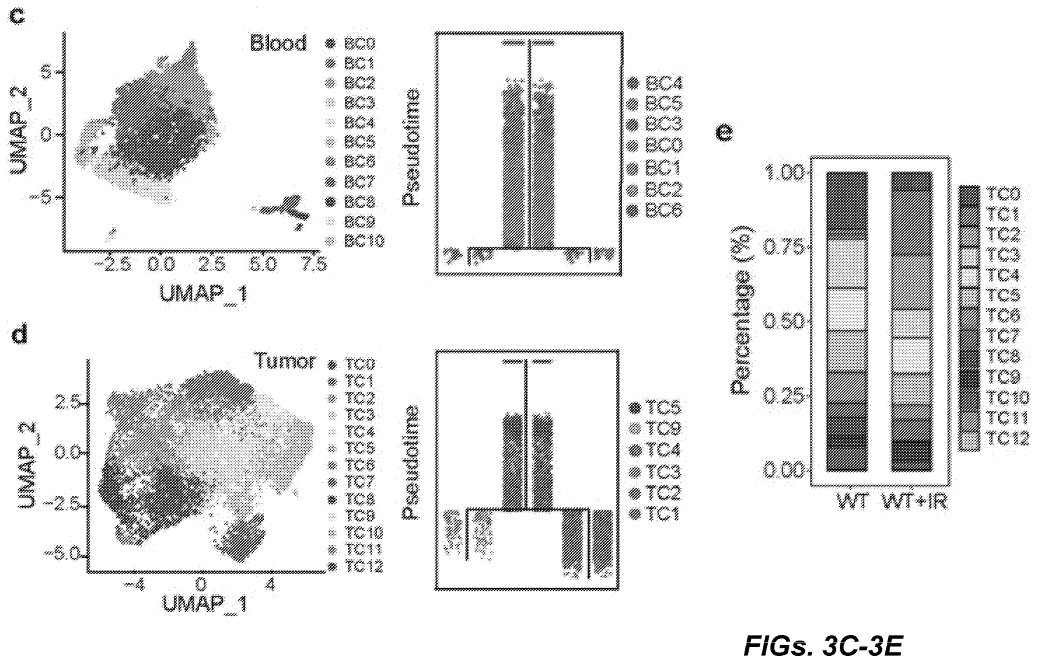

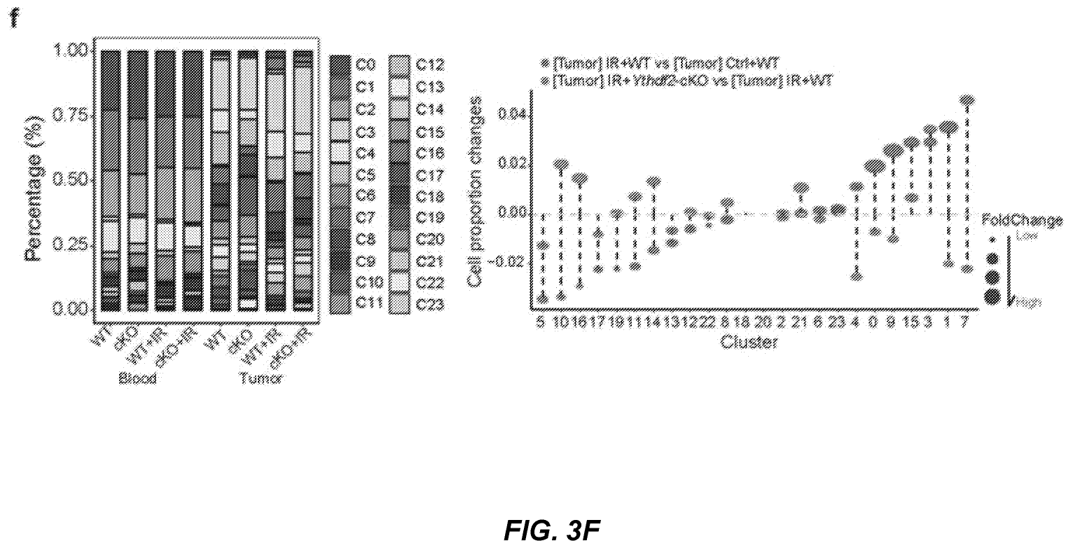

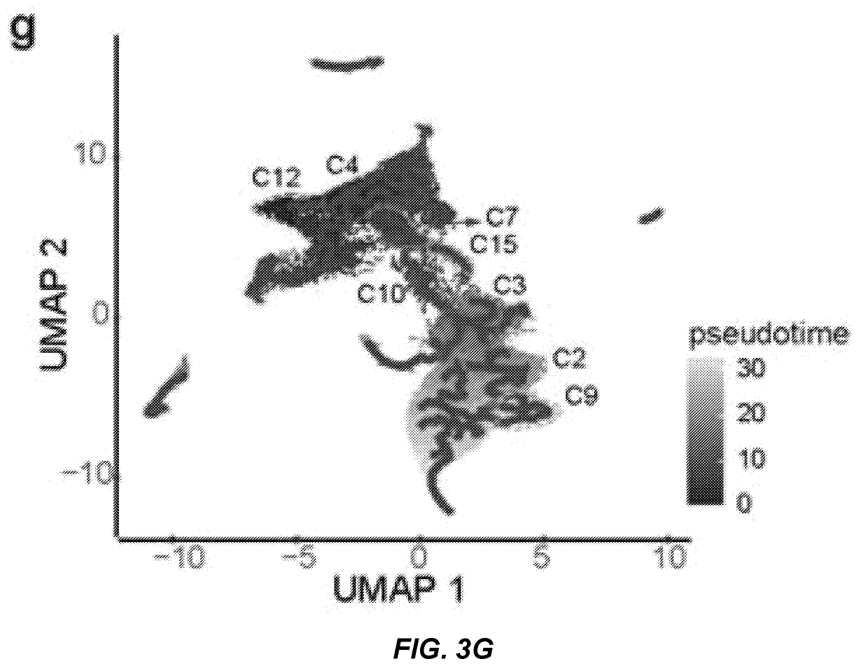

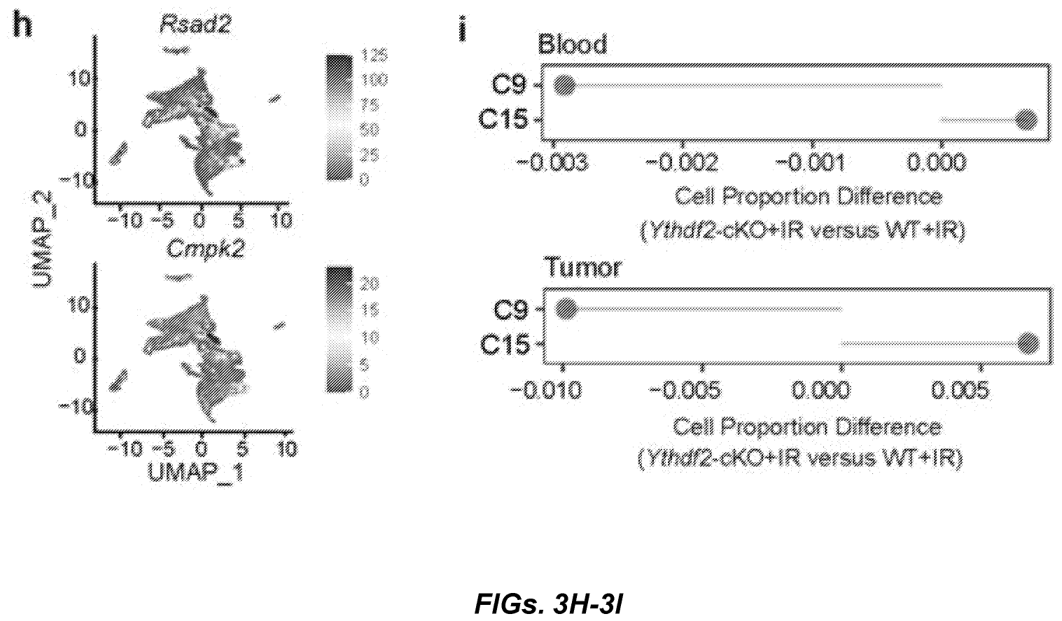

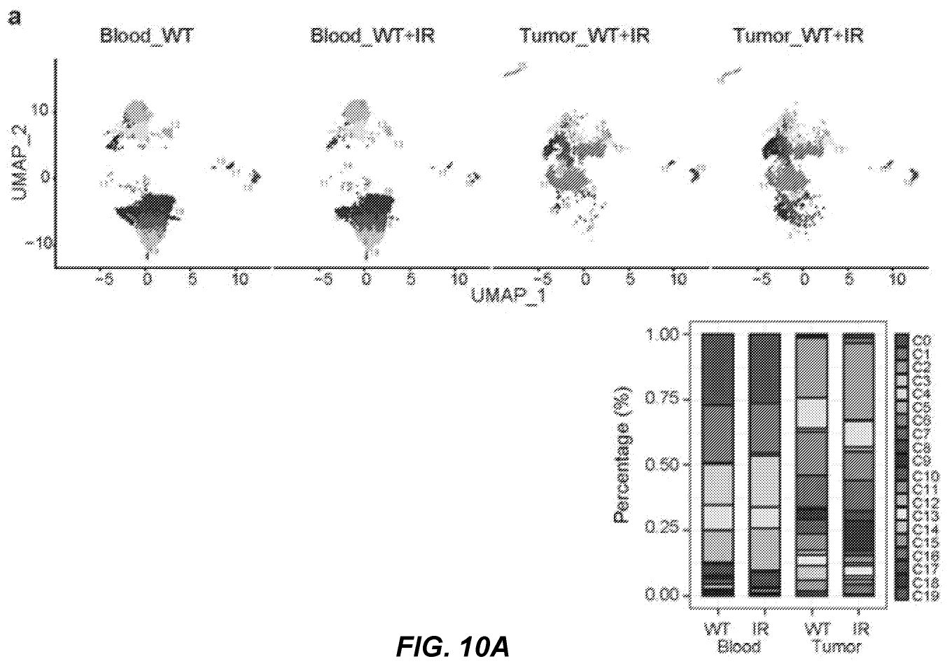

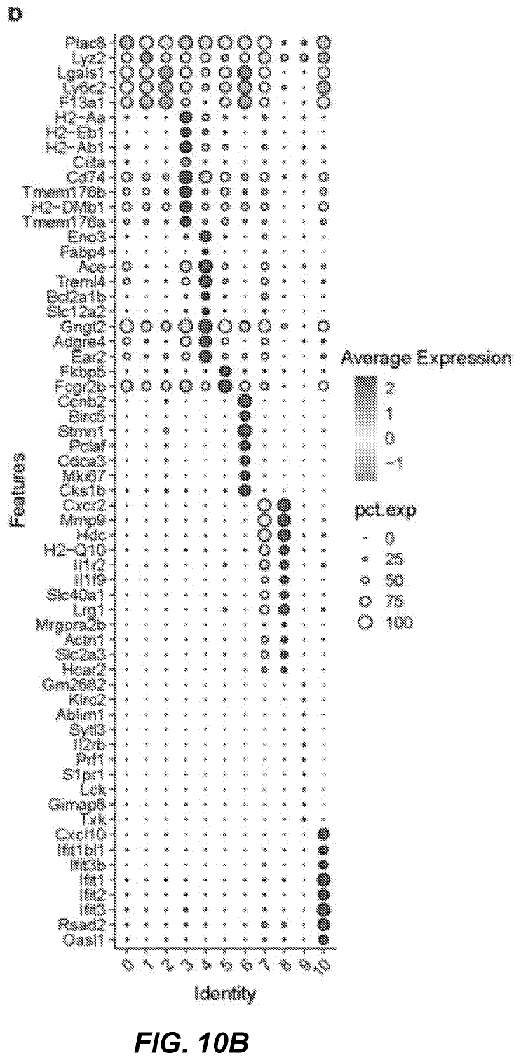

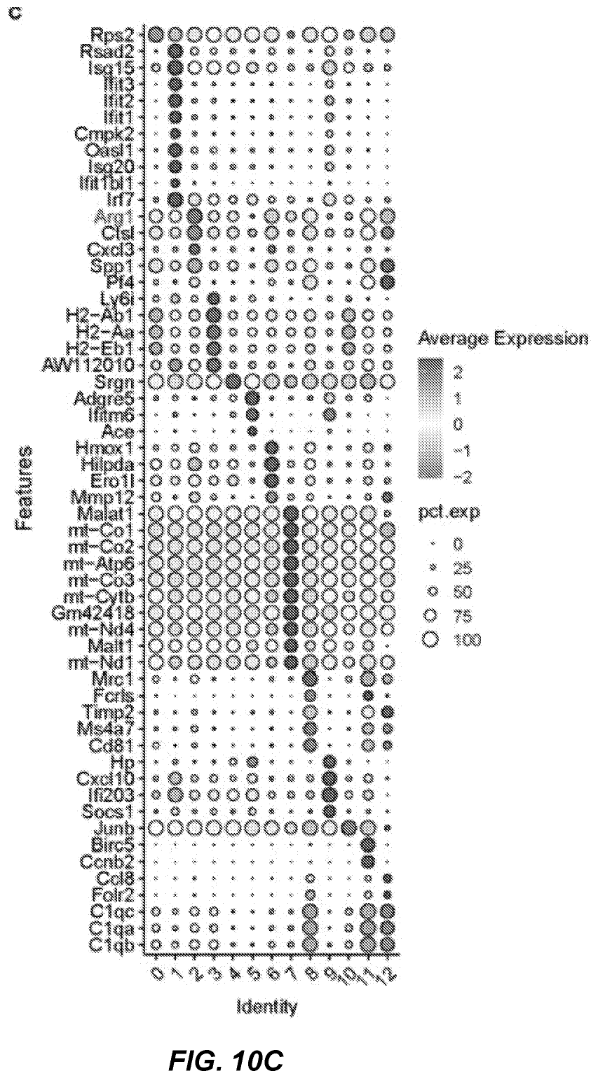

The inventors aimed to further delineate the effects of YTHDF2 on MDSCs in the context of IR. The inventors first investigated the state of monocytic MDSCs (mMDSC), which are immature myeloid cells, by performing scRNA-seq using CD45⁺CD11b⁺Ly6C^{hi} cells (mMDSC) isolated from blood and MC38 tumors of irradiated mice. To dissect mMDSC heterogeneity, the inventors applied unbiased clustering algorithms and identified 19 distinct cell populations belonging to four broad cell types: monocytes, macrophages, DCs, and neutrophils (FIG. 3A, FIG. 10A). In blood. IR consistently modulates mMDSC development. For example, C3 (Neutrophil-Csf3r) and C5 (Mono-Hopx) were increased in blood after IR; C9 (Mono-Rsad2) and C2 (Macro-Inhba) were increased in tumors after IR (FIG. 3B). Local tumor IR modulates mMDSC development intratumorally and systemically, demonstrating that local IR alters systemic immune responses.

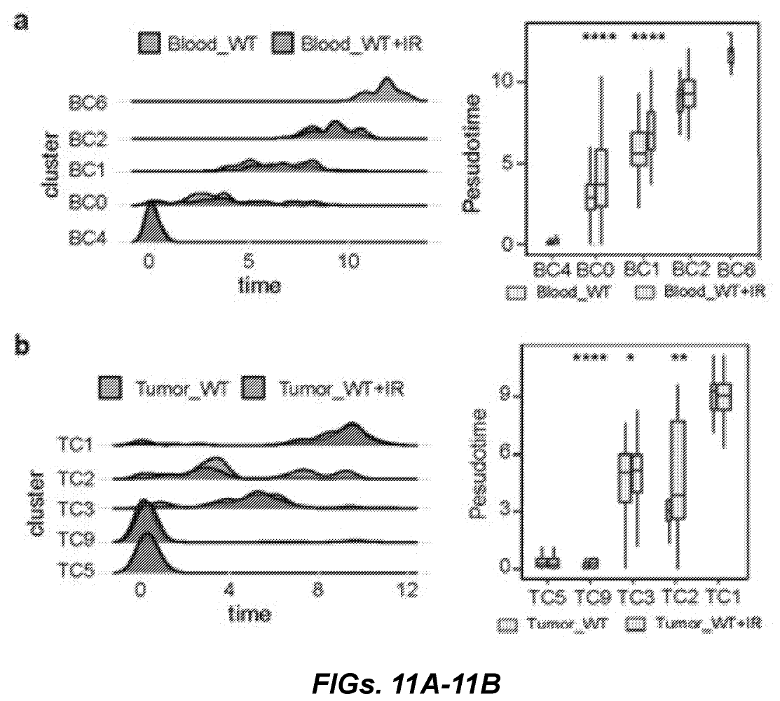

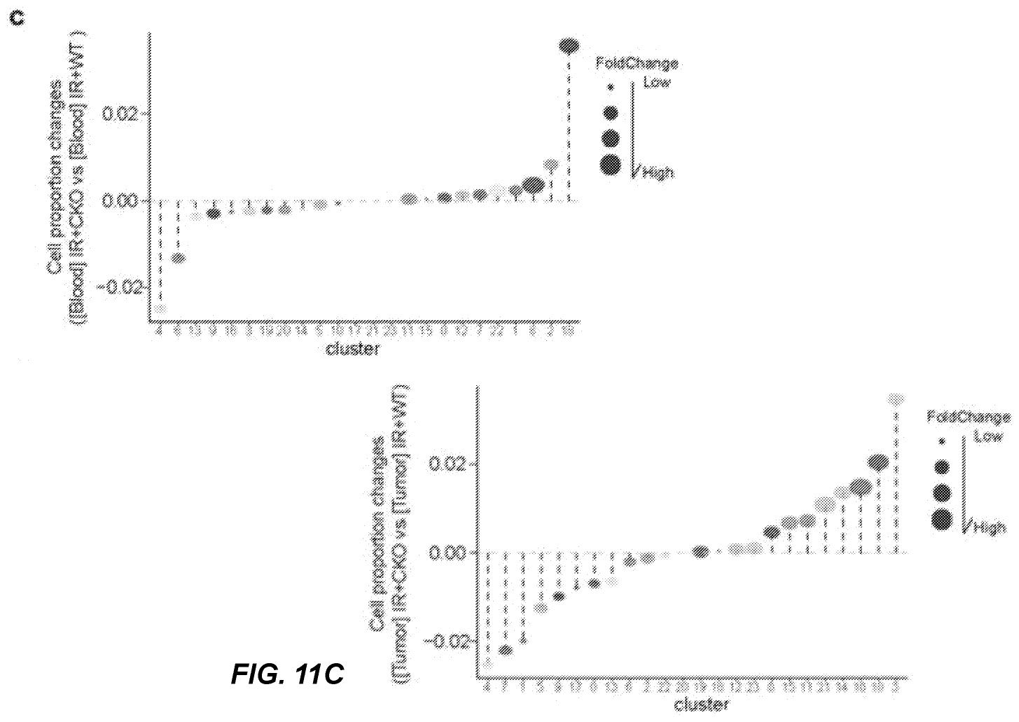

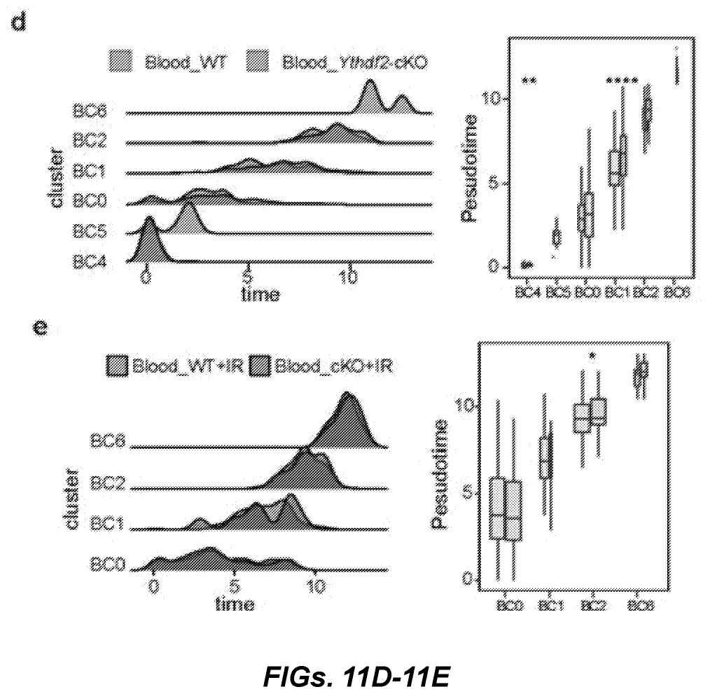

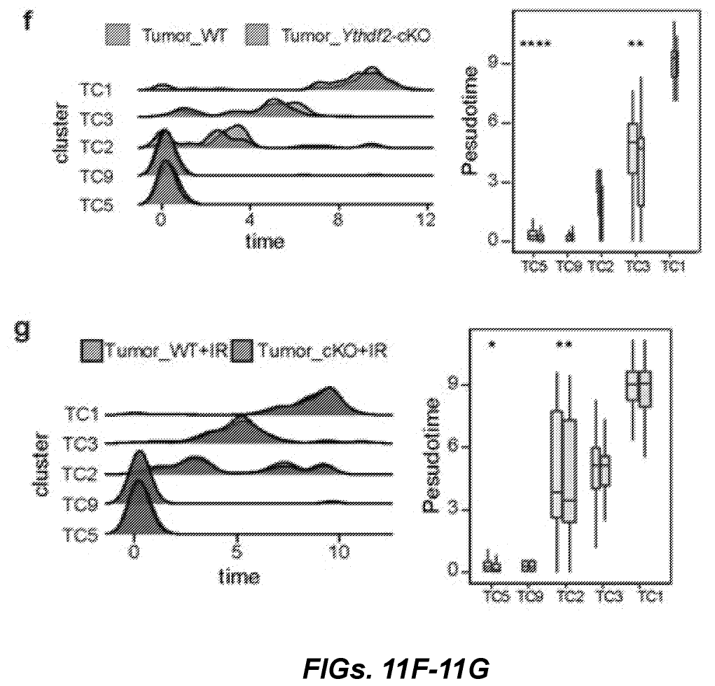

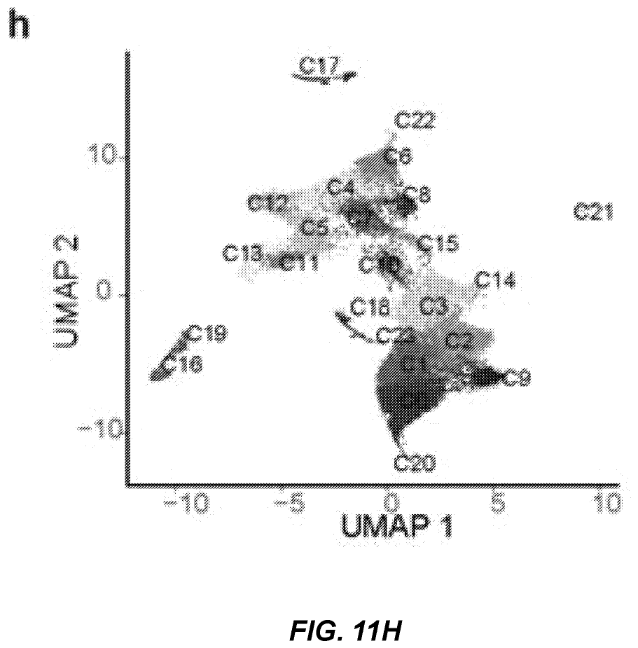

To infer the potential differentiation trajectories, the inventors ordered mMDSCs derived from blood along a pseudotime axis, and observed that mMDSC differentiation occurs on a tightly organized trajectory, starting from BC4 cluster, through BC0, BC1, BC2, and ending with the BC6 cluster (FIG. 3C). BC6 exhibits cluster-specific expression of Ccnb2, Birc5, Stmn1, Pclaf, Cdca3, Mki67, and Cks1b (FIG. 10B), indicative of highly proliferative activity. In tumors, cluster TC5 gradually develops into TC9, then TC1; or into TC2, and TC3 (FIG. 3D). Notably, the immunosuppressive gene Arg/was highly expressed in ten out of thirteen clusters, indicative of a suppressive phenotype (FIG. 10C). The proportion of TC1 and TC2 cells, as two major mMDSC populations in tumors, were increased following IR condition compared to non-IR condition (FIG. 3E). To investigate the role of IR in the differentiation of mMDSC in blood, the inventors next quantified the pseudotime of each cell subset in irradiated and unirradiated controls. The inventors found that BC0 and BC1 from irradiated mice showed increased pseudotime compared with unirradiated controls (FIG. 11A). The inventors also observed significantly increased pseudotime of TC9, TC2 and TC3 from irradiated mice versus unirradiated controls (FIG. 11B). Together, the inventors' findings reveal that tumor-local IR remodeled the landscape of mMDSC populations, possibly through accelerating mMDSC differentiation, and triggered a suppressive tumor microenvironment.

### Example 4—YTHDF2 Affects mMDSC Differentiation in the Context of IR

To assess the relationship between YTHDF2 inhibition and mMDSC differentiation, the inventors queried scRNA-seq data of mMDSCs from Ythdf2-cKO mice. Ythdf2 knockout led to changes in the proportion of distinct MDSC subsets in both blood and tumor in irradiated and unirradiated controls, compared with WT mice (FIG. 3F, FIG. 11C). First, the inventors conducted the trajectory analysis of mMDSCs in blood and observed that, in unirradiated mice, BC4 and BC1 showed increased pseudotime in Ythdf2-cKO mice compared with WT (FIG. 11D). In irradiated mice. BC2 showed increased pseudotime in Ythdf2-cKO mice compared with WT (FIG. 11E). Second, the inventors conducted the trajectory analysis of mMDSCs in tumor and found that TC5 and TC3 showed a significantly decreased pseudotime in Ythdf2-cKO compared with the WT (FIG. 11F). TC5 and TC2 showed decreased pseudotime in Ythdf2-cKO+IR compared with the WT+IR (FIG. 11G). Strikingly, the pattern of cell population changes in “Ythdf2-cKO+IR vs. WT+IR” is largely opposite to that in “WT+IR vs. WT” (FIG. 3F), suggesting YTHDF2 plays a key role in MDSC differentiation in response to IR. By establishing a single-cell atlas, the inventors demonstrated that Ythdf2 knockout alters the mMDSC differentiation and the effects are amplified by IR.

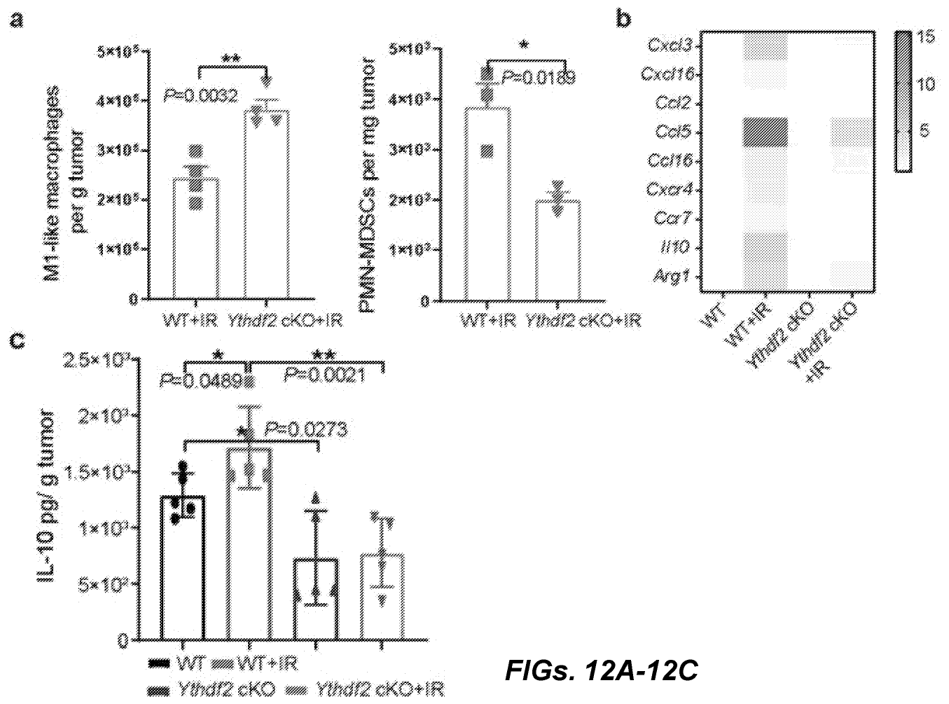

MDSCs migrate from blood to tumor in response to radiotherapy. To depict a continuous picture of mMDSC differentiation, the inventors conducted the trajectory analysis in mMDSCs combining all samples (blood and tumor from WT or Ythdf2-cKO+/−IR treatment) (FIG. 3G, FIG. 11H). The inventors identified C12 as monocyte precursor on the basis of abundant expression of Ear2²¹. The C12 population evolves into C4, and then C7, followed by branching into two separate paths: 1) C15 and 2) from C10, C3, C2, to C9 (FIG. 3G). C15 is classified as an M1-like macrophage, characterized by high levels of Rsad2 and Cmpk2 (FIG. 3H). C3, C2, and C9 were characterized as polymorphonuclear-MDSCs (PMN-MDSCs), consistent with a concept that mMDSCs can differentiate into PMN-MDSCs²². The percentage of C15 (M1-like cells) significantly increased, while C9 (PMN-MDSC-like cells) significantly decreased in IR treated Ythdf2-cKO mice versus IR treated WT mice (FIG. 3I), as further confirmed by flow cytometry analysis (FIG. 12A); this data mirrors the tumor growth phenotypes. These modifications of mMDSC clusters resulted in reprogramming of the host immune microenvironment locally and systemically in favor of enhanced anti-tumor immunity in Ythdf2-cKO mice in response to IR.

### Example 5—YTHDF2 Controls MDSC Migration and Suppressive Function in the Context of IR

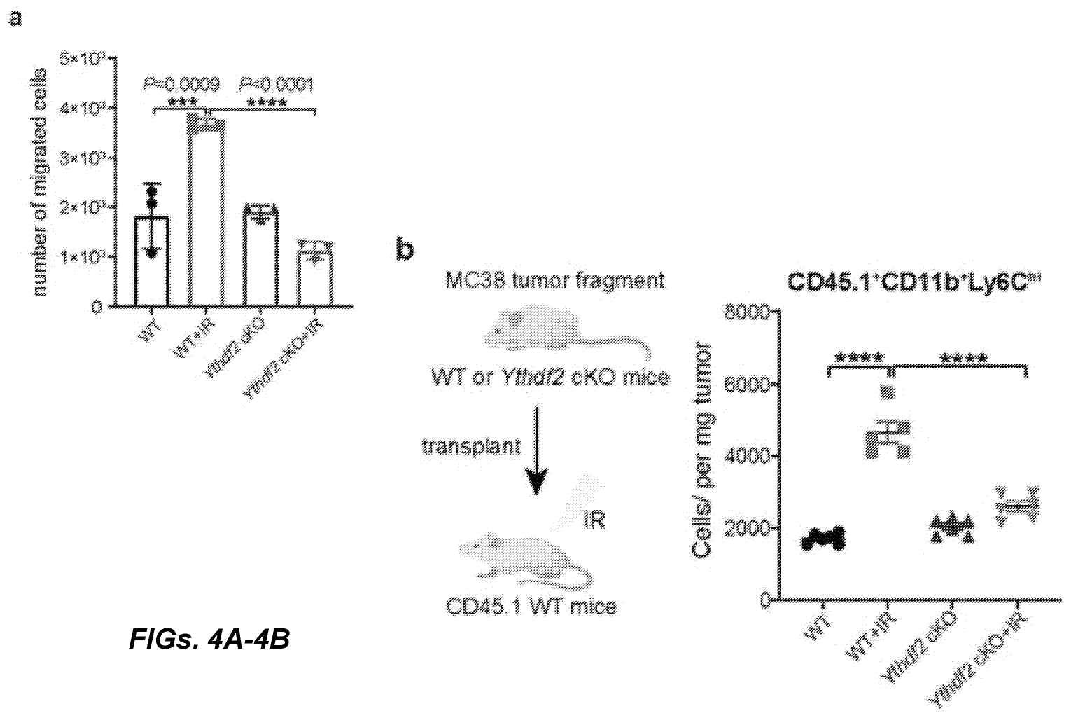

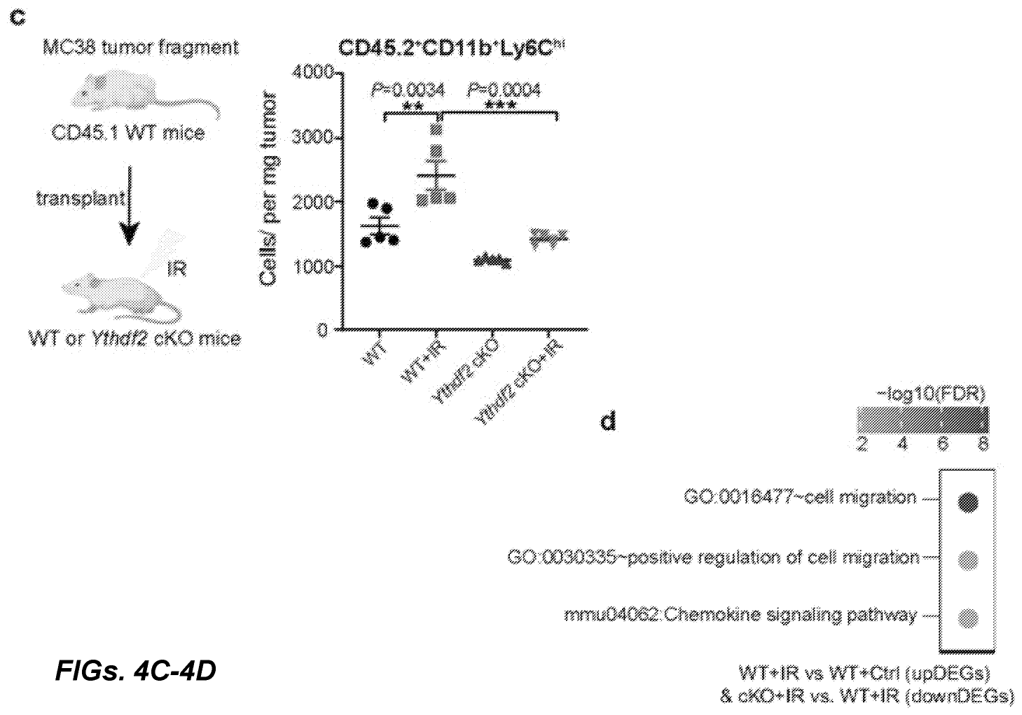

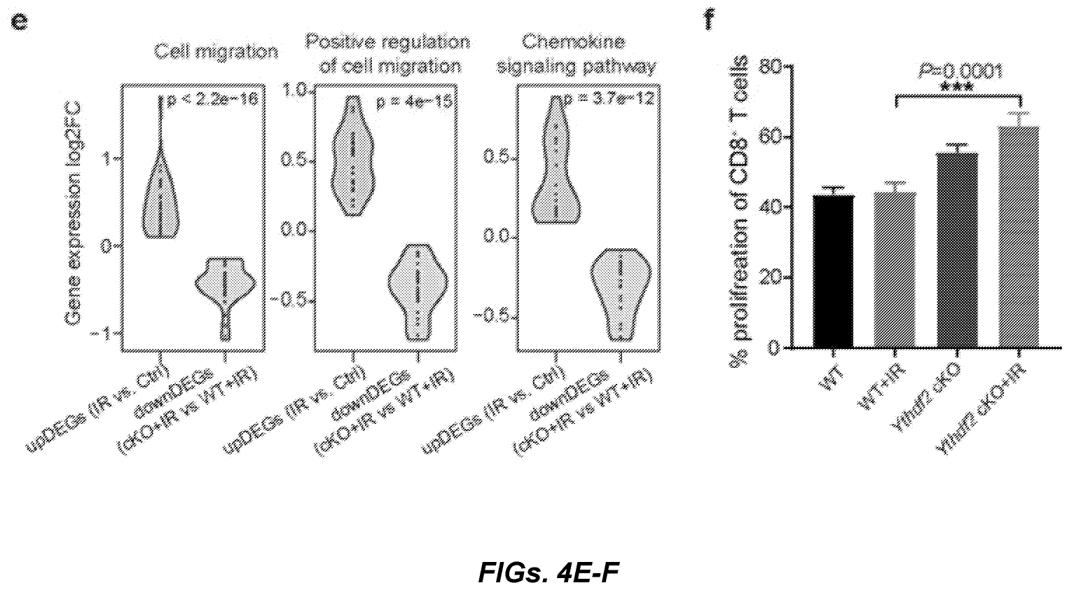

Having demonstrated that YTHDF2 affects MDSC differentiation in the context of IR, the inventors next investigated the role of YTHDF2 in IR-induced MDSC migration. Ythdf2 deletion impaired the migratory capacity of MDSCs as evidenced by a migration assay in which MDSCs in irradiated tumors of Ythdf2-cKO mice showed a significantly lower migration compared with that of WT mice (FIG. 4A). To further verify this, MC38 tumor fragments, which contain pre-existing MDSCs from tumors grown in WT or Ythdf2-cKO mice (both of which are CD45.2), were harvested and inoculated into CD45.1 WT mice. Three days after IR, there was no significant increase of CD45.1⁺CD11b⁺Ly6C^{hi} (mMDSC) cells in irradiated tumors compared with non-irradiated tumors from Ythdf2-cKO mice, whereas a significant increase was observed in WT tumor fragments post-IR (FIG. 4B). Further, the inventors observed consistent changes in chemokine expression in the infiltrating MDSCs (FIG. 12B). These results suggest that loss of Ythdf2 in myeloid cells abrogated IR-induced enhanced chemokine production to further attract MDSCs. The inventors also inoculated MC38 tumor fragments grown in CD45.1 mice into WT or Ythdf2-cKO mice (CD45.2) and found that IR also failed to induce the accumulation of CD45.2⁺MDSCs in tumors in Ythdf2-cKO mice (FIG. 4C), suggesting that Ythdf2-cKO MDSCs were less effective in trafficking to the tumor following IR. Taken together, the inventors' results indicate that deletion of Ythdf2 in the myeloid compartment leads to defects in both migration capacity and chemoattraction of MDSCs following IR.

For validation of these findings, the inventors performed mRNA-seq using MC38 tumor-infiltrating CD11b⁺ myeloid cells in WT and Ythdf2-cKO mice+/−IR. As expected, the GO enrichment analysis indicated that three pathways, including those affecting cell migration, chemokine signaling, and positive regulation of cell migration, were up-regulated in WT+IR versus WT+ctrl, and were down-regulated in Ythdf2-cKO+IR versus WT+IR (FIGS. 4D-E). These findings support the inventors' observation of MDSC migration phenotypes in vivo.

In terms of suppressive function, the inventors observed that, in the context of IR, tumor-infiltrating Ythdf2-cKO MDSCs exhibited attenuated suppressive function during co-culture with activated naïve CD8⁺ T cells, compared with WT MDSCs (FIG. 4F). In pursuing this further, the inventors investigated the expression levels of proteins produced by MDSCs that mediate immune suppression. IR significantly induced IL-10 production and Arg1 expression in tumors in WT mice compared with Ythdf2-cKO mice (FIGS. 12B-C). Taken together, these results reveal that Ythdf2 knockout impairs both MDSC migration and suppressive functions, which may be critical to the enhanced antitumor effect of IR observed in Ythdf2-cKO.

### Example 6—NF-κB/RELA Mediates Radiation-Induced YTHDF2 Expression in MDSCs

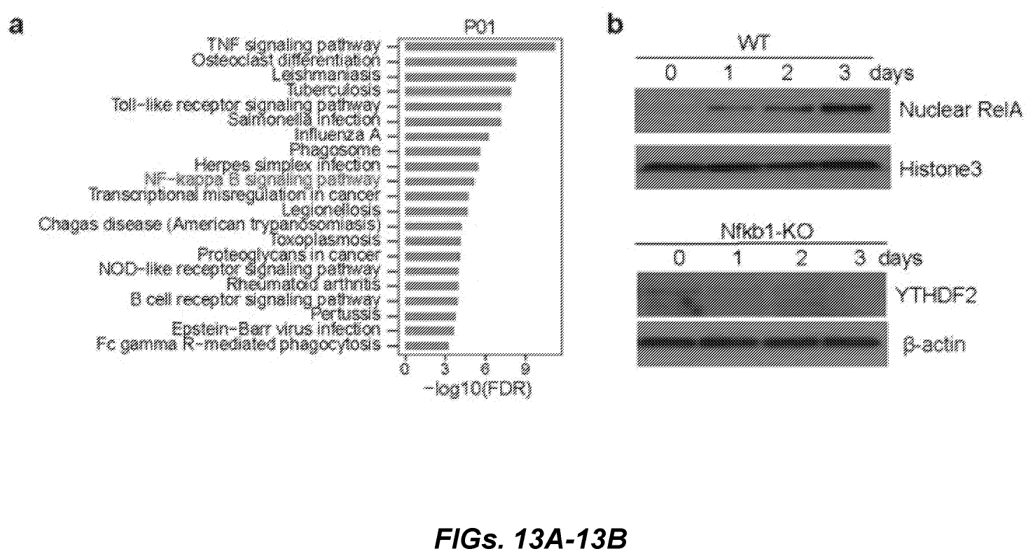

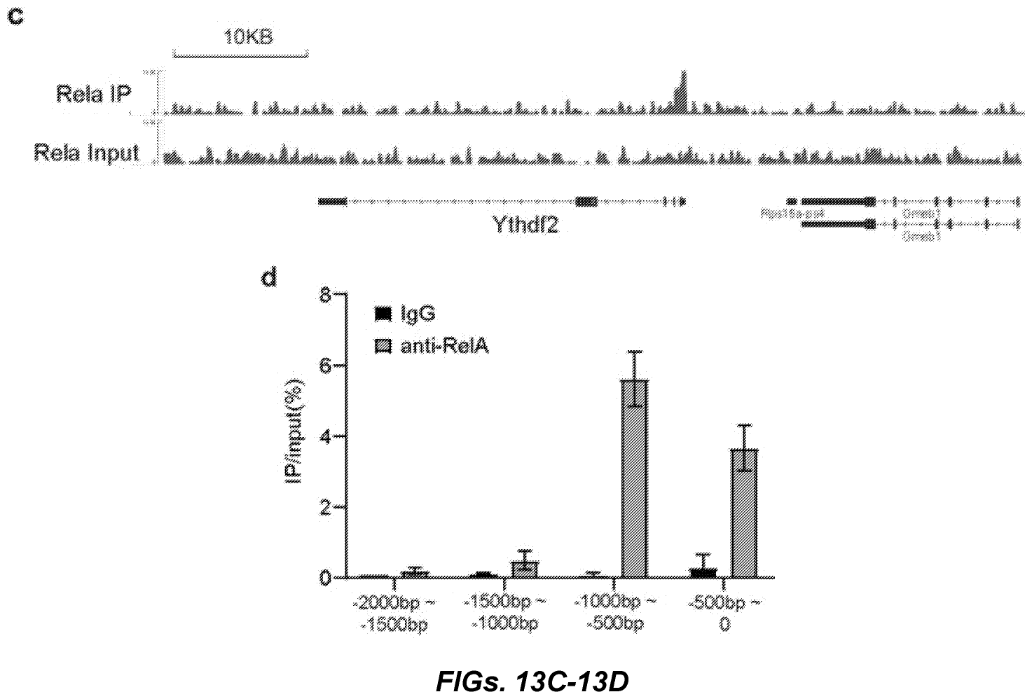

The inventors sought to investigate the potential mechanisms involved in radiation induction of YTHDF2 in MDSCs. First, the inventors performed functional enrichment analysis with genes differentially expressed in “monocytic MDSC_Ly6c2” (P01 population, FIG. 1A) following IR treatment, and found that genes of the NF-kappa B (NF-κB) signaling pathway are enriched in this population post-IR (FIG. 13A). These data are consistent with previous findings that IR activates NF-κB²³⁻²⁵. Further investigation of the relationship between NF-κB and YTHDF2 revealed that the level of nuclear RELA is increased after IR (FIG. 13B). YTHDF2 expression was not induced by IR in MDSCs deficient of Nfkb1 (FIG. 13B), demonstrating that NFKB1 is required for IR induction of YTHDF2. NFKB1 is an important component in NF-κB signaling by forming a RELA/NFKB1 heterodimer required for RELA nuclear translocation²⁶. The inventors next analyzed a public dataset of RELA chromatin immunoprecipitation sequencing (ChIP-seq) conducted in mouse bone marrow-derived macrophages and found the predicted direct binding between RELA and the Ythdf2 promoter region (FIG. 13C). To verify this finding, the inventors performed ChIP coupled with quantitative PCR (ChIP-qPCR) analysis using bone marrow-derived CD11b⁺Ly6C⁺ cells. The results revealed that RELA indeed directly binds to the Ythdf2 promoter region (˜1.0-2.0 kb proximal to the transcription start site) (FIG. 13D). Collectively, these findings indicate that IR upregulates YTHDF2 expression via the NF-κB/RELA signaling pathway.

### Example 7—IR-Induced YTHDF2 Enhances NF-κB Signaling by Promoting m⁶A-Modified RNA Degradation

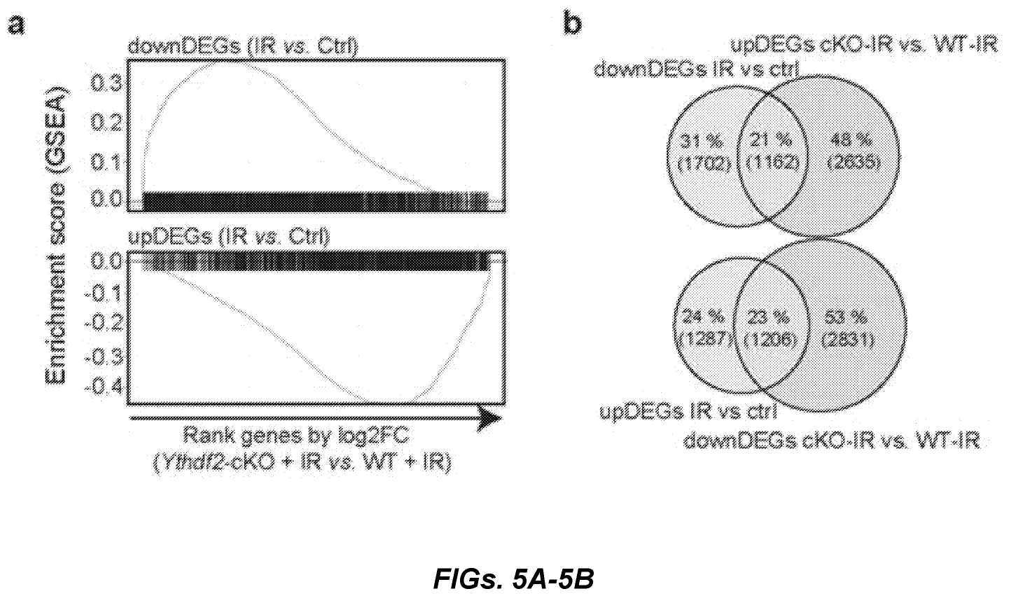

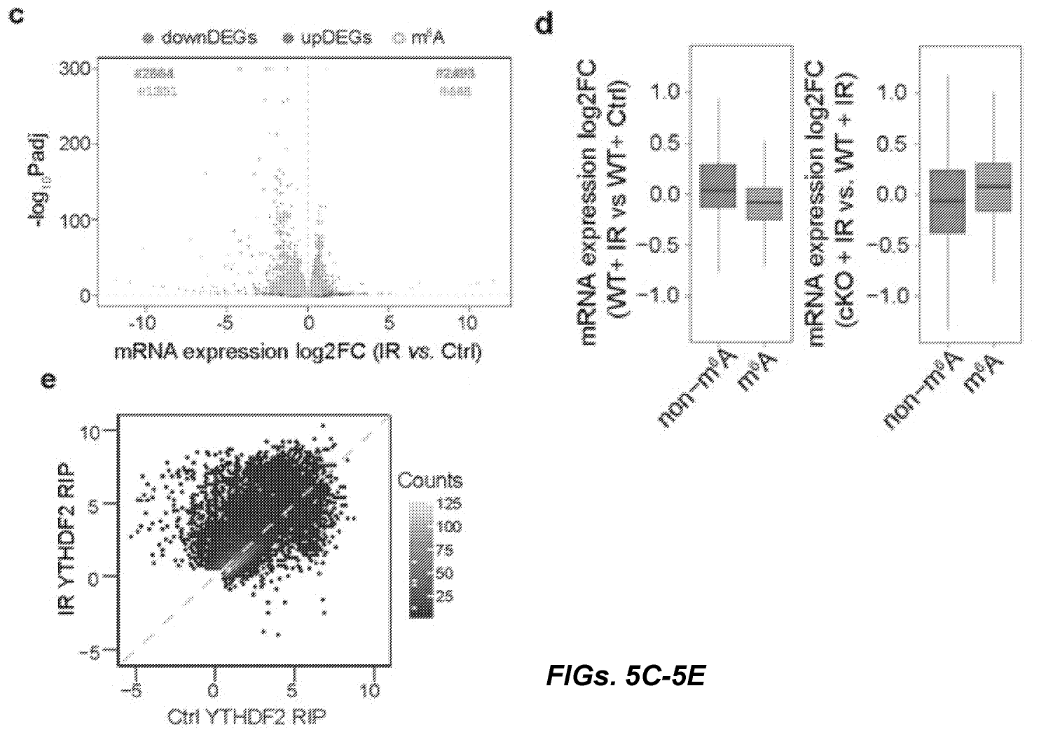

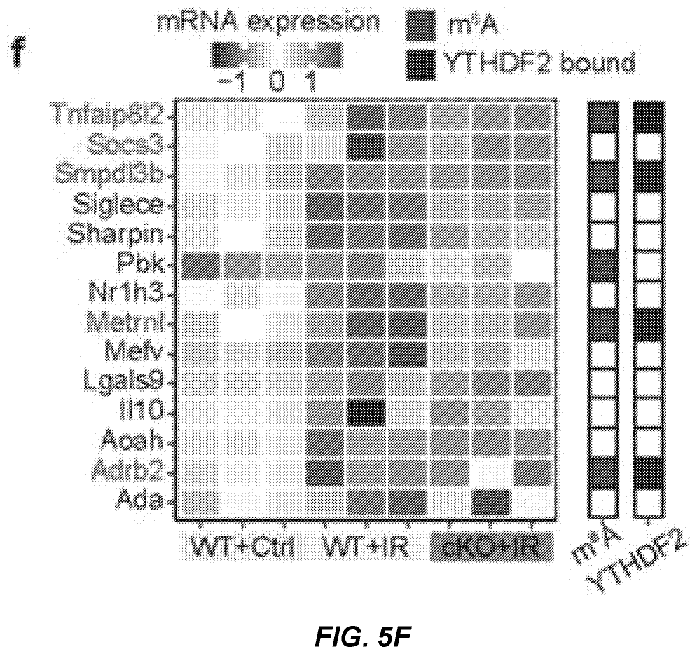

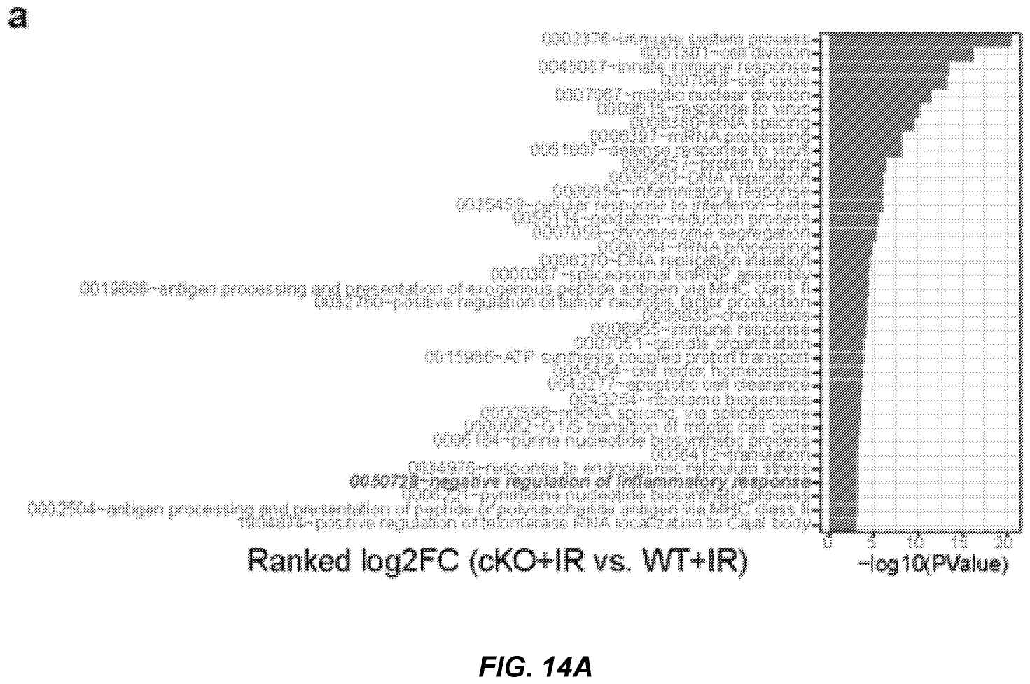

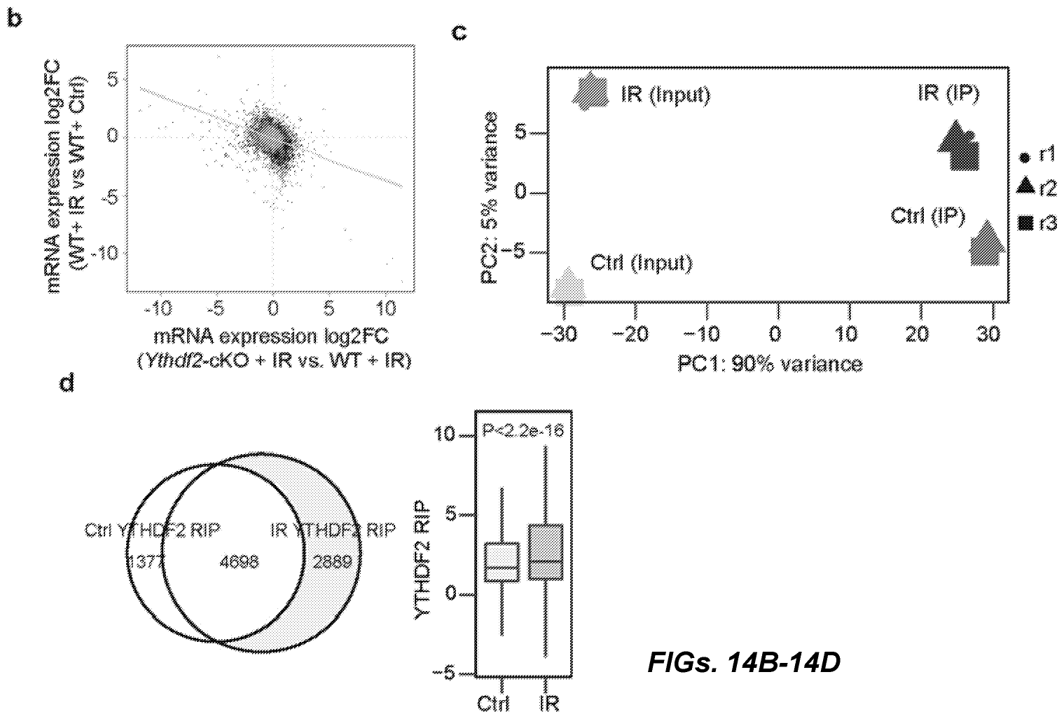

To investigate the molecular mechanisms of YTHDF2 function in MDSCs in the context of IR, the inventors reanalyzed the mRNA-seq of MC38 tumor-infiltrating CD11b⁺ myeloid cells in Ythdf2-cKO mice with IR or unirradiated controls. The inventors analyzed the gene expression profiles and found that knockout of Ythdf2 abolished the transcriptional changes induced by IR alone (FIG. 5A). The inventors focused on the differentially expressed genes, comparing IR versus non-IR in WT mice and also IR versus non-IR in Ythdf2-cKO mice (FIG. 5B) to perform gene enrichment analysis. The inventors found an enrichment of the “negative regulation of inflammatory response” pathway in Ythdf2-cKO+IR (FIG. 14A), containing five genes (Tnfaip8l2, Socs3, Smpdl3b, Metrnl and Adrb2) which have been reported as negative regulators for NF-κB signaling²⁷⁻³¹, which facilitates MDSC migration and chemokine/cytokine regulation³². Thus the inventors' data indicate that IR induces YTHDF2 via NF-κB, and the elevated YTHDF2 levels may in turn enhance NF-κB signaling in MDSCs, thus forming an IR/YTHDF2/NF-κB circuit. To test this hypothesis, the inventors sought to characterize the downstream direct targets of YTHDF2 by performing N⁶-methyladenosine-sequencing RNA immunoprecipitation followed by high-throughput sequencing (MeRIP-seq) and RNA immunoprecipitation sequencing (RIP-seq). The inventors observed that a majority of m⁶A-marked genes were down-regulated after IR treatment (1351 down-regulated genes versus 448 up-regulated genes) (FIG. 5C) and Ythdf2 deletion reversed this suppression (FIG. 5D, FIG. 14B). Furthermore, with YTHDF2 expression being elevated by IR, the number of YTHDF2-bound transcripts increased accordingly (FIG. 5E, FIGS. 14C-D), suggesting that increased YTHDF2 binding post IR mediated the decrease of mRNA abundance.

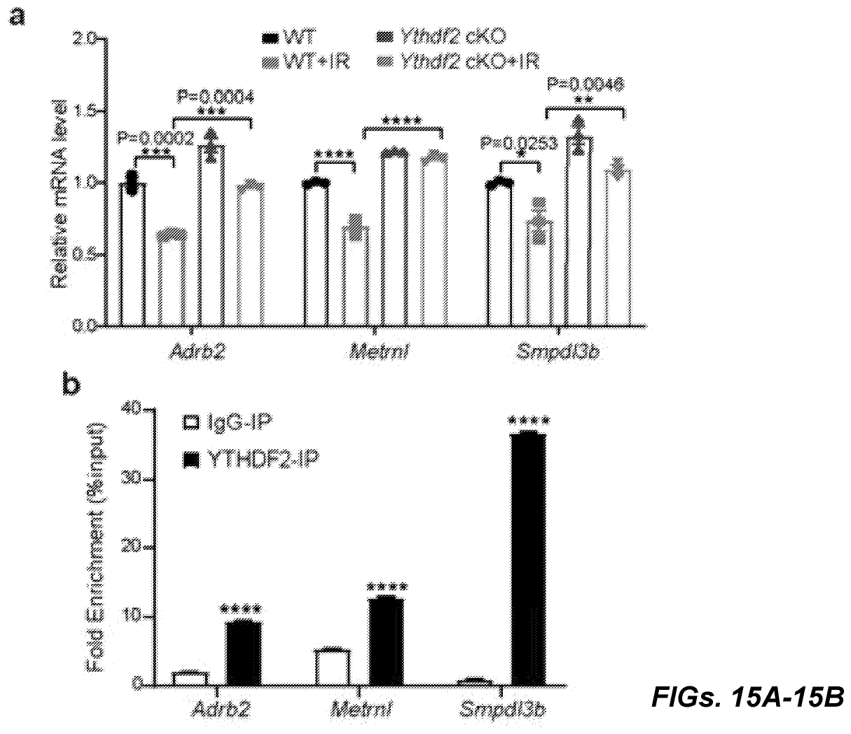

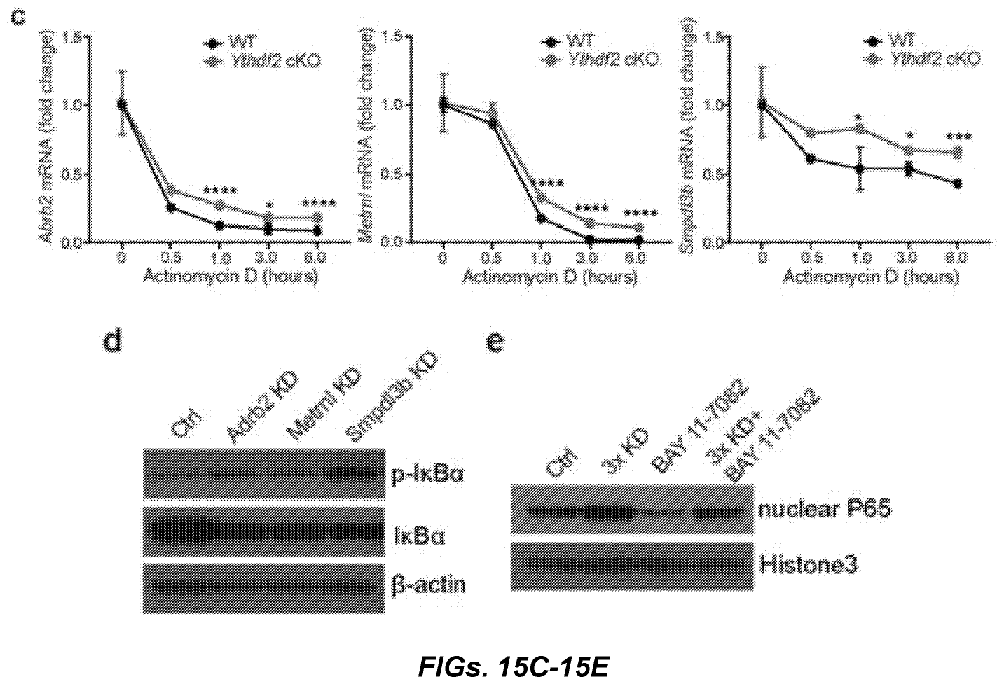

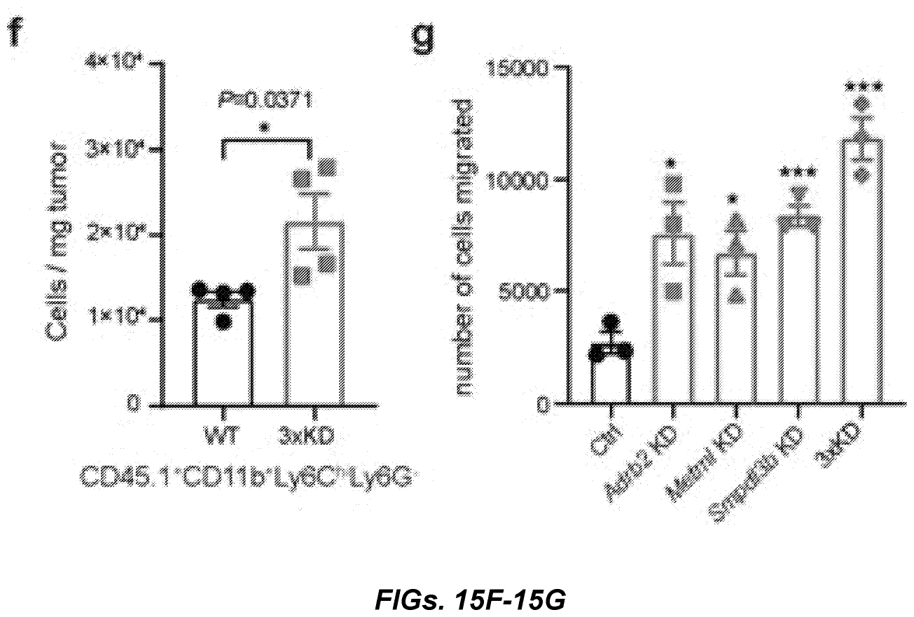

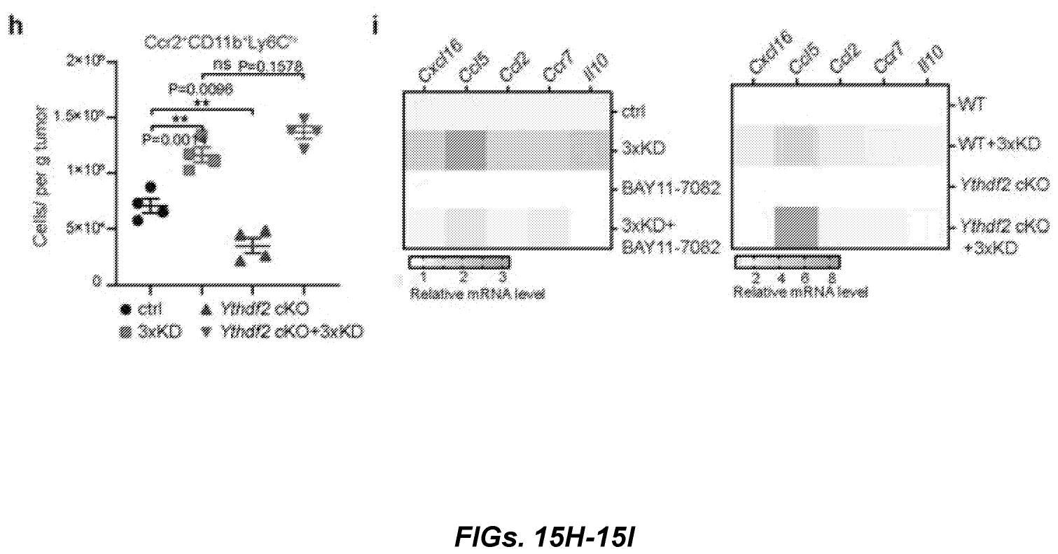

Close investigation of the above-mentioned genes as negative regulators of NF-κB signaling revealed three (Adrb2, Metrn1, and Smpdl3b) genes that are m⁶A marked and also targeted by YTHDF2 (FIG. 5F. FIGS. 15A-B). Additionally, their transcript half times were increased in Ythdf2-cKO (FIG. 15C), consistent with the known function of YTHDF2 in promoting mRNA degradation²⁰. The inventors hypothesized that, in the context of IR, Ythdf2 enhances NF-κB signaling through reducing the expression of Adrb2, Metrnl, or Smpdl3b. To test this, the inventors generated BM-MDSCs with all 3 genes knocked down (3×KD) and conducted western blot assay for NF-κB signaling. The inventors observed increased levels of IκBα phosphorylation and nuclear localization of RELA in knockdown MDSCs (FIGS. 15D-E). Moreover. IκBα phosphorylation inhibitor (BAY 11-7082)³³ treatment prevented this increase (FIG. 15E). These results indicate that the m⁶A/YTHDF2 axis regulates NF-κB signaling in MDSCs by targeting negative regulators of the NF-κB pathway. To assess whether the IR/YTHDF2/NF-κB axis contributes to MDSC migration or function, the inventors employed Ccr2-KO mice in which the radiation-induced MDSC infiltration into tumors is markedly decreased¹¹. The inventors adoptively transferred 3×KD-MDSC into MC38 tumor-bearing Ccr2-knockout mice and observed that compared with the transferred WT MDSCs. 3×KD-MDSCs elicited significantly higher migration capacities post-IR (FIG. 15F). In vitro migration assays of 3×KD MDSCs also showed similar results (FIG. 15G). To further confirm the involvement of YTHDF2 in radiation-induced MDSC infiltration, the inventors performed the aforementioned transfer experiment using 3×KD-Ythdf2-cKO MDSCs. As expected, three days after IR, the level of infiltrating MDSCs (Ccr2⁺CD11b⁺Ly6C^{hi}) was restored to a level similar to 3×KD MDSCs transfer (FIG. 15H). Functionally, the inventors observed consistent changes in the expression of genes associated with migration and function of MDSCs, including Ccl2, Ccl5, Cxcl16, Ccr7, and Il10 (FIG. 15I). Collectively, these data demonstrate that the enhanced migration and function of MDSCs induced by IR rely on the expression of YTHDF2, most likely via enhanced activation of the NF-κB pathway. Together with the previous finding that IR-mediated YTHDF2 induction relies upon NF-κB activation, the inventors propose that an YTHDF2/NF-κB positive feedback loop governs migration and suppression of MDSCs and tumor extrinsic radioresistance.

### Example 8—Pharmacological Inhibition of YTHDF2 Enhances Responses to Radiotherapy and Immunotherapy

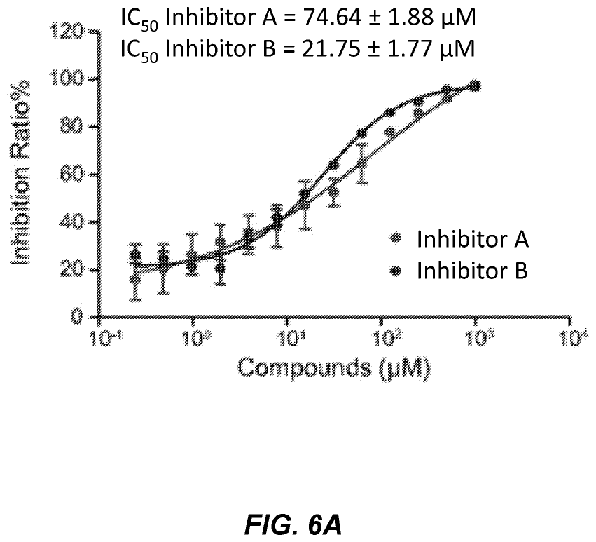

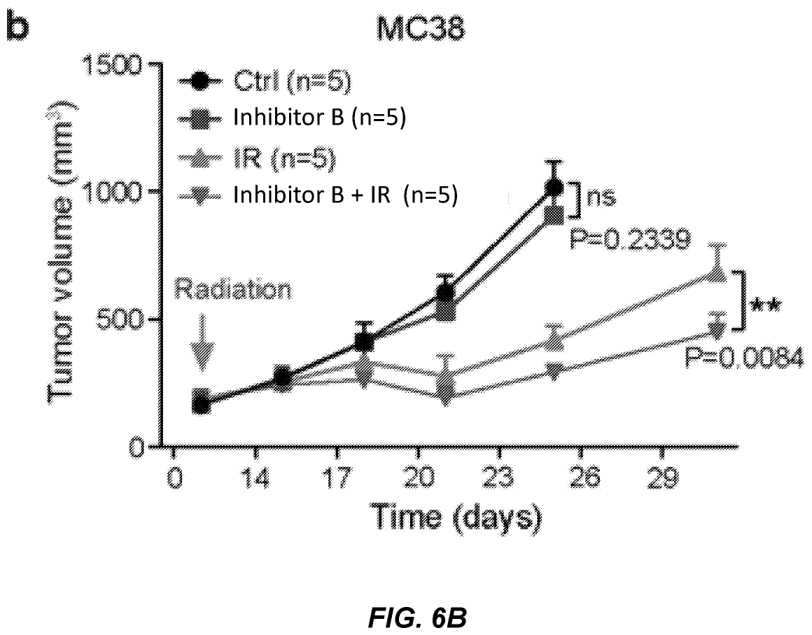

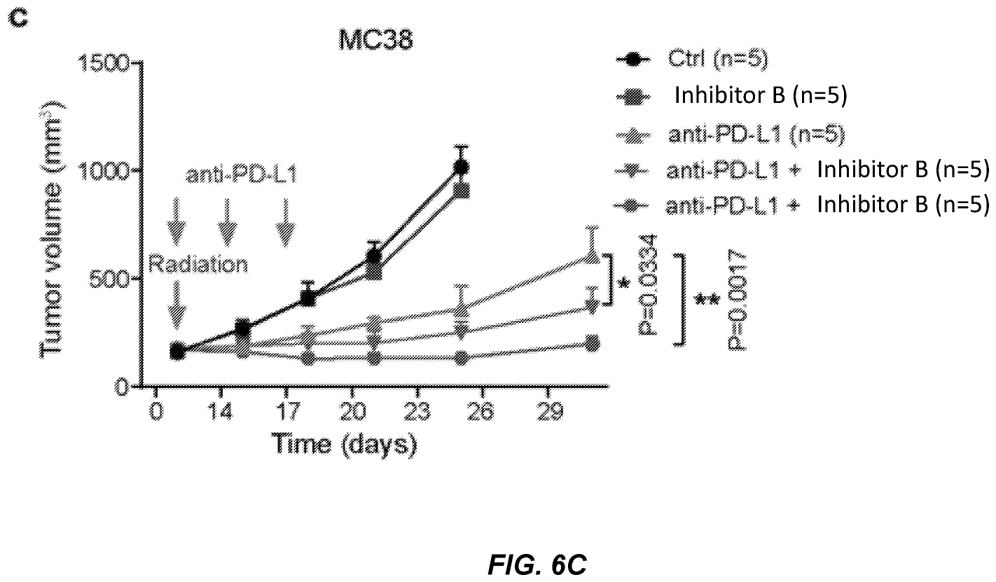

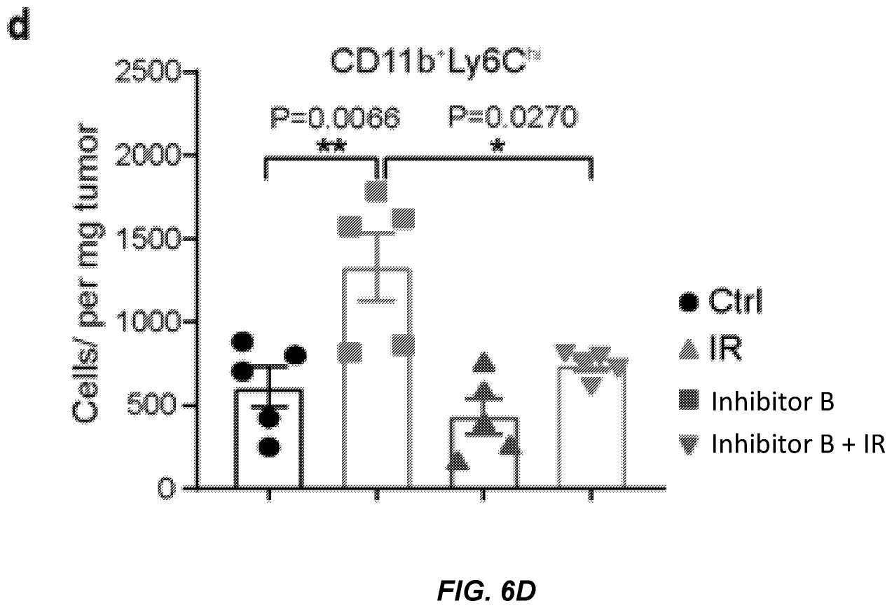

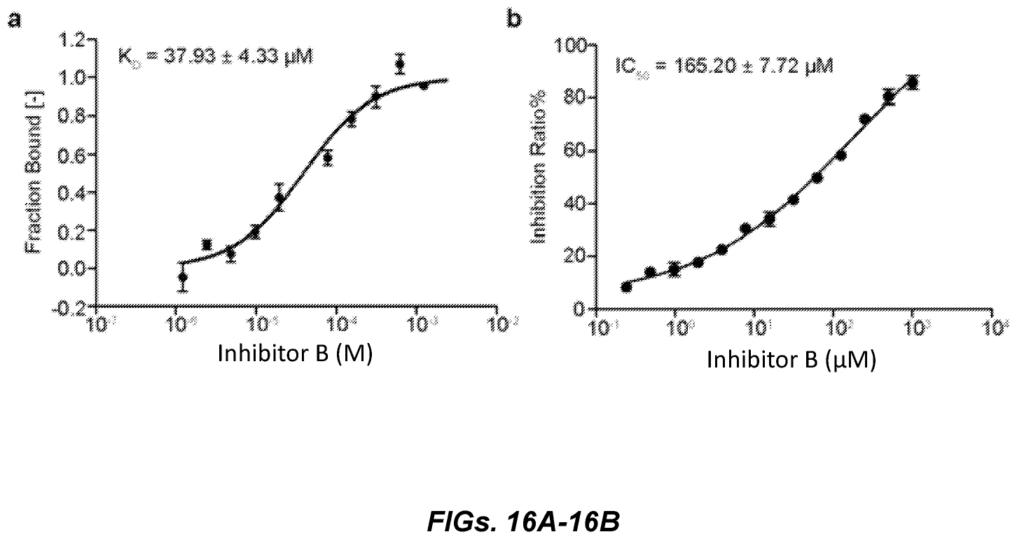

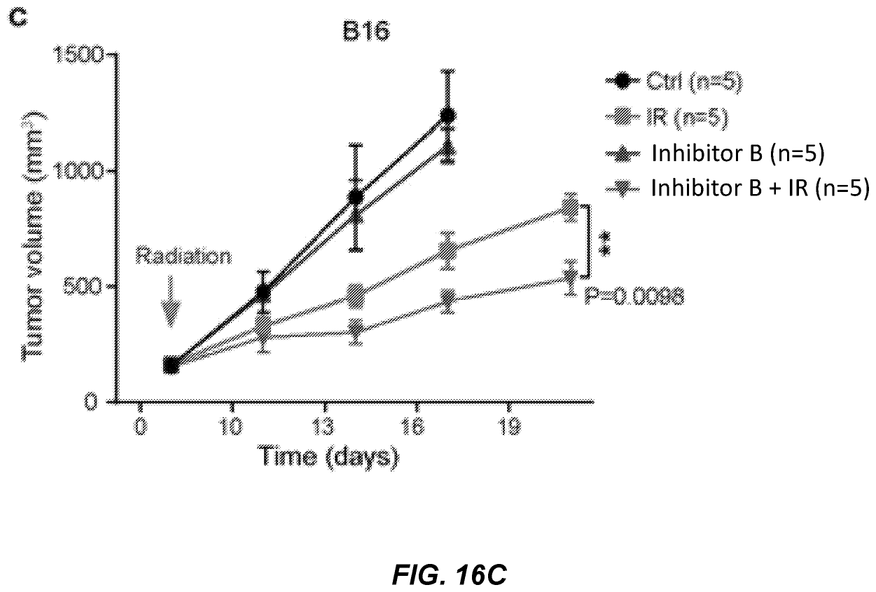

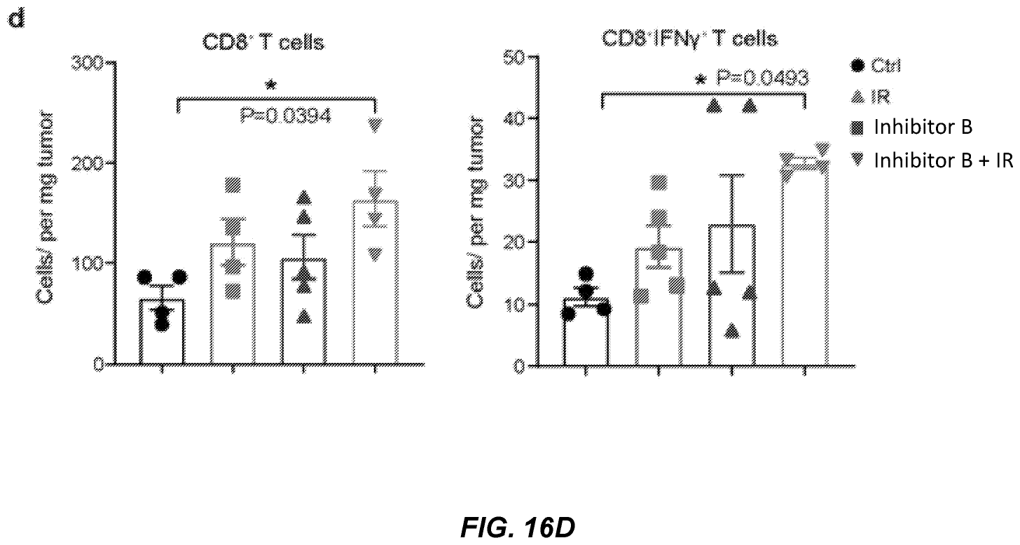

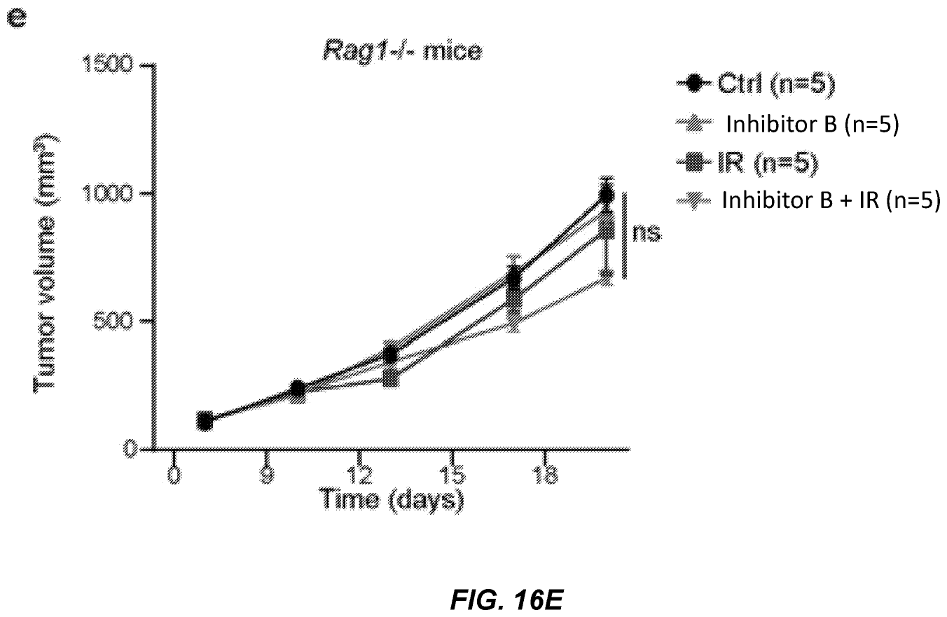

To demonstrate that RT+YTHDF2 blockade can be translated into a clinically relevant strategy, the inventors used Inhibitor A and Inhibitor B as inhibitors of YTHDF2. Inhibitor A inhibits YTHDF2 binding to m⁶A-containing RNA with an IC50 of 74.6±1.9 μM measured via AlphaScreen assay (FIG. 6A). Inhibitor B had a measured IC50 of 21.8±1.8 μM (FIG. 6A). To further assess the capacity of Inhibitor B binding to YTHDF2, the inventors conducted a microscale thermophoresis assay and confirmed that Inhibitor B binds to YTHDF2 with a binding constant (KD) of 37.9±4.3 μM (FIG. 16A). The inventors next explored the inhibitory activity of Inhibitor B against YTHDF1, another member of the YT521-B homology (YTH) domain-containing proteins family to evaluate its selectivity. Inhibitor B obstructs the interaction between YTHDF1 and m⁶A with an IC50 of 165.2±7.7 μM, as shown in FIG. 16B, suggesting that Inhibitor B exhibits a preference for inhibiting YTHDF2 binding to m⁶A-modified RNA.

To investigate whether Inhibitor B improves the response to IR at a similar level as Ythdf2 genetic deletion, tumor-bearing mice were treated daily with Inhibitor B starting on the day of IR treatment. Consistent with results obtained in Ythdf2-cKO, Inhibitor B treatment alone did not inhibit tumor growth in either the MC38 or B16 murine models. However, when combined with IR treatment, Inhibitor B treatment significantly enhanced tumor growth inhibition of IR compared with IR alone (FIG. 6B, FIG. 16C). The inventors also investigated whether Inhibitor B can further increase efficacy of IR and anti-PD-L1 treatment using the MC38 model. Compared with any single treatment group, combination treatment with Inhibitor B and anti-PD-L1 resulted in significantly slower growth of MC38 tumors, and the triple therapy of Inhibitor B, IR, and anti-PD-L1 gave rise to the most robust antitumor effects (FIG. 6C).

To interrogate the underlying immunological mechanisms, the inventors profiled the tumor-infiltrating immune cells in MC38 tumors following Inhibitor B treatment by flow cytometry. The inventors observed that IR failed to increase infiltration of CD11b⁺Ly6C^{hi} cell in tumors with Inhibitor B treatment, whereas a 2-fold increase was observed following IR alone (FIG. 6D). This is consistent with the inventors' aforementioned findings in irradiated Ythdf2-cKO mice. Given the critical roles of MDSCs in determining T-cell infiltration and function, the inventors examined the CD8⁺ T-cell populations in tumors five days after IR and Inhibitor B treatment. The total numbers of both CD8⁺ T cells and IFNγ⁺CD8⁺ T cells were increased in tumors receiving the combination treatment (FIG. 16D). This was also evidenced by the abolished antitumor efficacy of Inhibitor B plus IR in Rag1 knockout mice (FIG. 16E). The results demonstrate that the antitumor effect of the combination treatment with Inhibitor B and IR relies on adaptive immunity and is similar to that observed in Ythdf2 deleted mice.

### Example 10—YTHDF2 Regulates Survival and Migration of Tregs

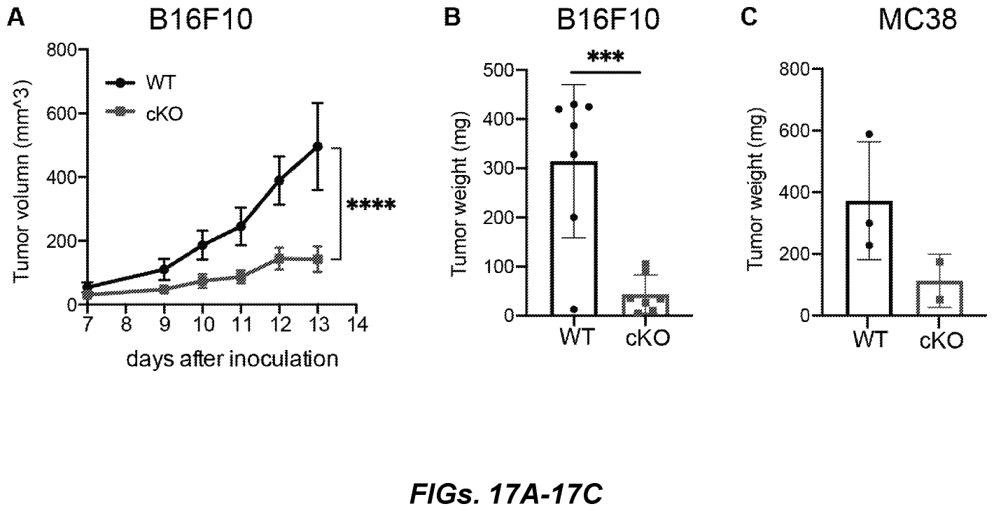

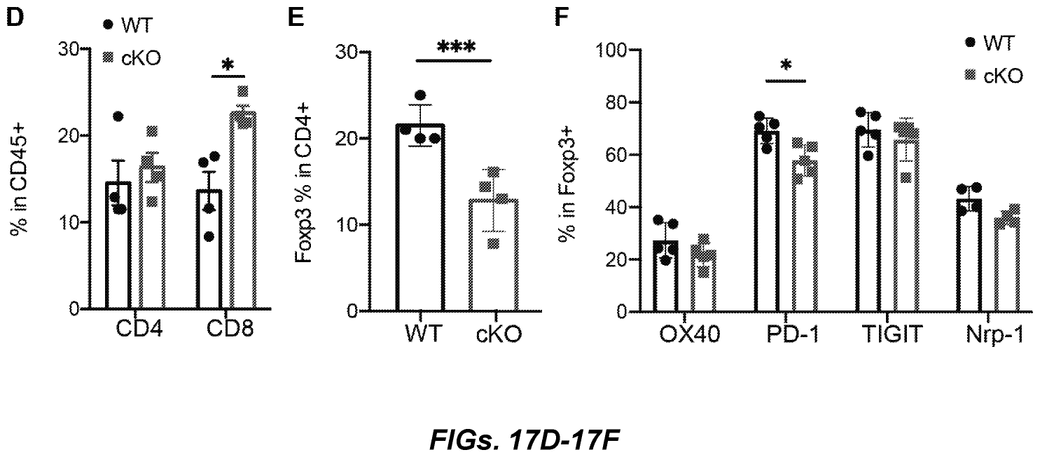

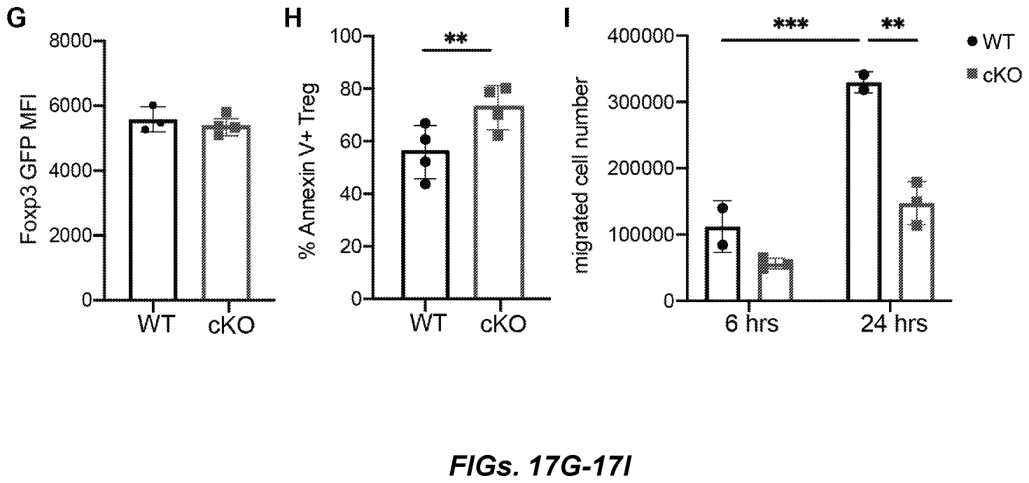

To explore the cause of decreased Treg infiltration, the effect of YTHDF2 on Treg stability, generation, and recruitment was examined. Both splenic and tumor-infiltrating Tregs had the same Foxp3 expression level from WT and cKO mice (FIG. 17G). Furthermore, in vitro stimulation of Tregs with various cytokines showed no difference in Foxp3 stability in the absence of YTHDF2. In vitro induction of Tregs with TGFβ showed no defects without YTHDF2, implying that, in some aspects, Tregs are still induced within the tumor. However, the lack of YTHDF2 led to higher apoptosis of tumor-infiltrating Tregs (FIG. 17H). YTHDF2 KO Tregs were also less migrative towards tumor culture media in vitro (FIG. 17I). While splenic Tregs retained T cell homeostasis in cKO mice, they could not fully suppress T cells in a co-transplant experiment. In some aspects, in the tumor environment, YTHDF2 KO Tregs are less suppressive.

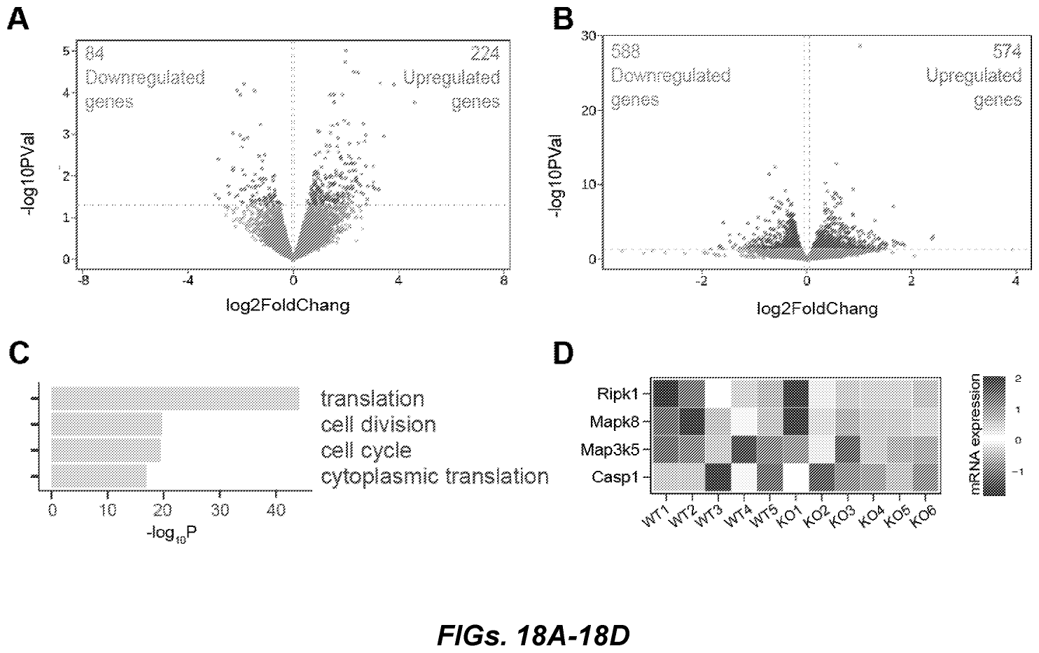

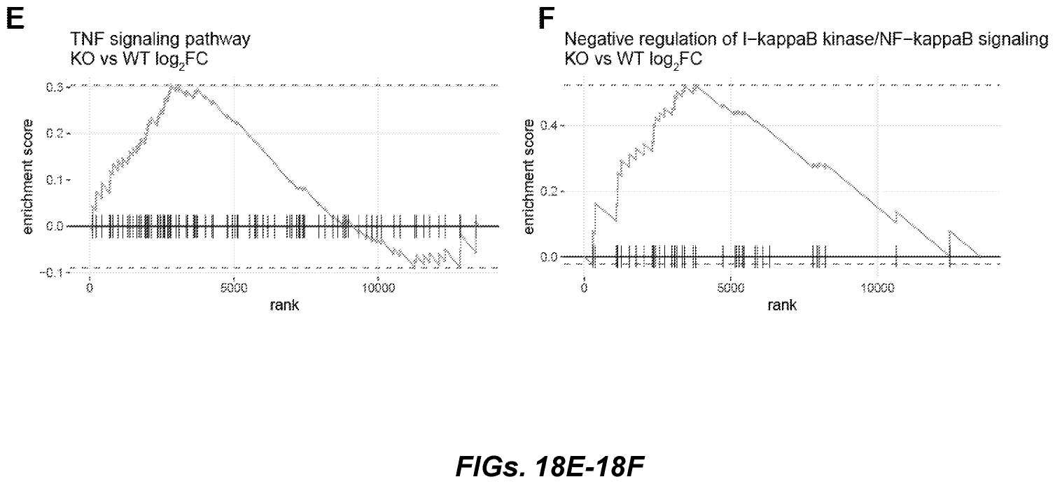

RNA-sequencing of tumor-infiltrating Tregs and splenic Tregs from WT and cKO mice revealed key pathways and functions that were impacted by YTHDF2. It was confirmed that splenic Tregs and tumor-infiltrating Tregs were largely different, and the tumor microenvironment further differentiated WT and YTHDF2 KO Tregs, leading to more differentially expressed genes (FIGS. 18A, 18B).

In tumor Tregs, the down-regulated genes enriched for cell division and cell cycle (FIG. 18C). Meanwhile, apoptosis-related genes are upregulated after YTHDF2 deletion (FIG. 18D). These changes in expression correlate with the higher apoptosis rate (FIG. 17H). The upregulated genes also enriched the TNF signaling pathway (FIG. 18E). While TNF signaling typically activates the NF-κB signaling pathway, a group of NF-κB negative regulators was also upregulated (FIG. 18F). Activation of NF-κB can initiate negative feedback signals, and in some aspects, loss of YTHDF2 can lead to dysregulation of the feedback regulation. TNFβ-induced signaling through TNFR2 (Tnfrsf1b) is can support Treg function and survival through activation of NF-κB, but the opposite is observed for TGFβ induced Tregs (iTregs). Tumor microenvironment often has a high level of TGFβ and TNFα, which could have a negative feedback effect on iTregs without YTHDF2's regulation.

### Example 11: Local Tumor Irradiation Increases Tumor-Associated Myeloid Cells Expressing YTHDF2

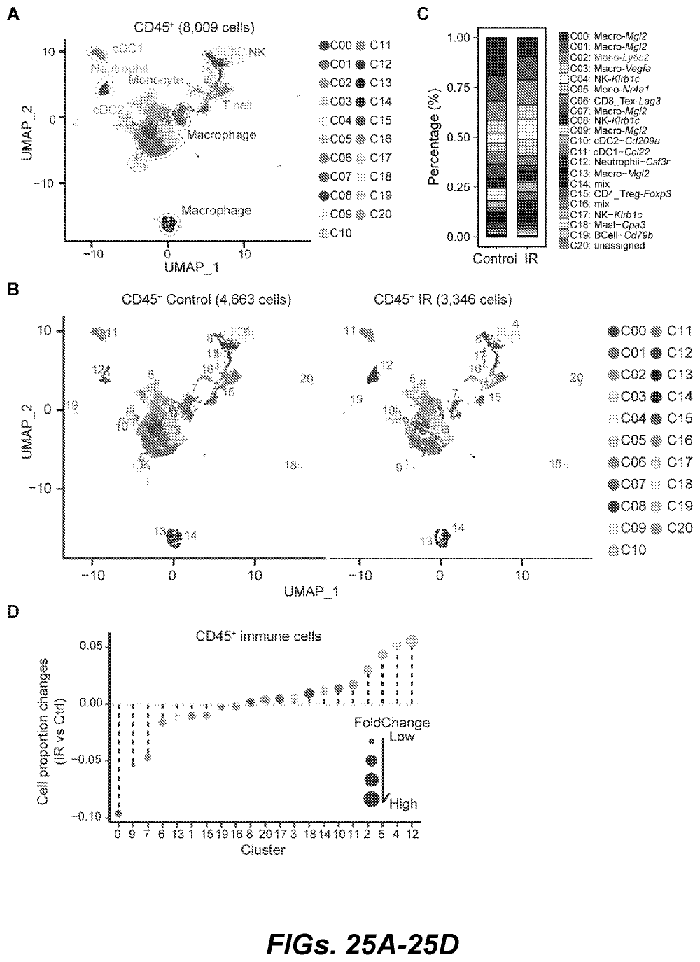

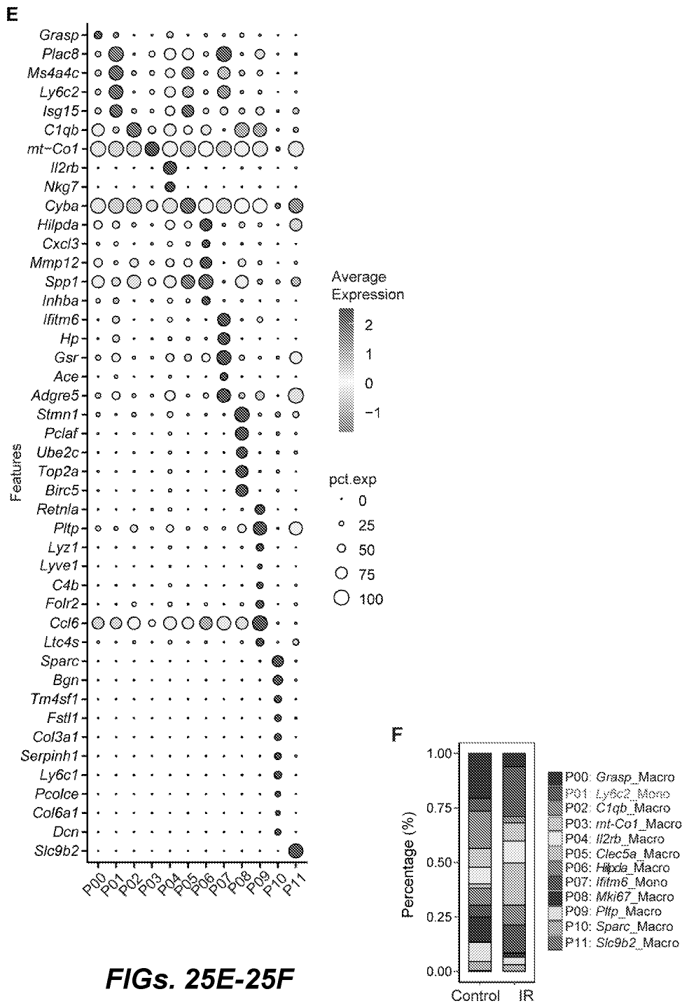

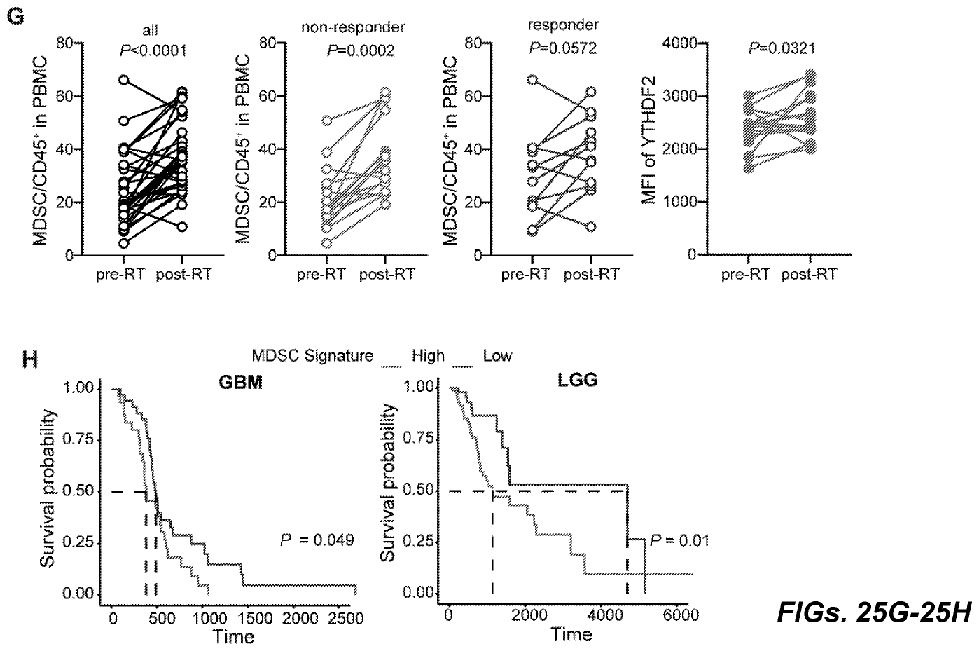

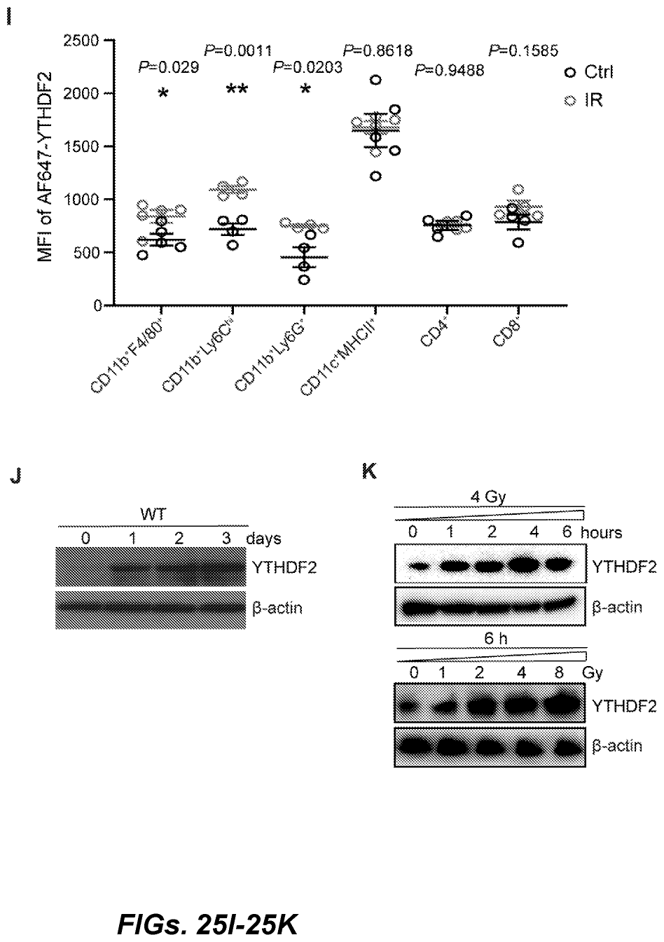

To investigate the cellular and molecular contexture of the tumor immune microenvironment (TME) following high-dose radiation used in ablative radiotherapy, the inventors characterized CD45⁺ immune cells isolated from irradiated (4 days after 20 Gy-IR) and non-irradiated MC38 tumors by high-throughput single-cell RNA sequencing (scRNA-seq). The inventors identified five major cell lineages including T cells, natural killer (NK) cells, dendritic cells (DCs), monocytes, and macrophages, based on gene expression signatures (FIG. 25A). The inventors then characterized the changes of these cell subtypes in irradiated tumors compared with non-irradiated tumors. The proportion of T cells was slightly decreased, while an NK subset (Klrb1c_NK, C04), two DC subsets (Ccl22_cDC1, C11 and Cd209a_cDC2, C10), distinct subsets of macrophages and monocytes (C03 and C05), and neutrophil subsets (C12) were increased post IR (FIGS. 25B-25D). Of note, C03 cells showed upregulated Vegfa expression, and C05 cells showed upregulated Nr4a1 expression, suggesting their tendency toward the M2 phenotype. Thus, IR markedly changes the TME in ways that alter tumor-infiltrating immune cells including NKs, DCs, macrophages, and neutrophils.

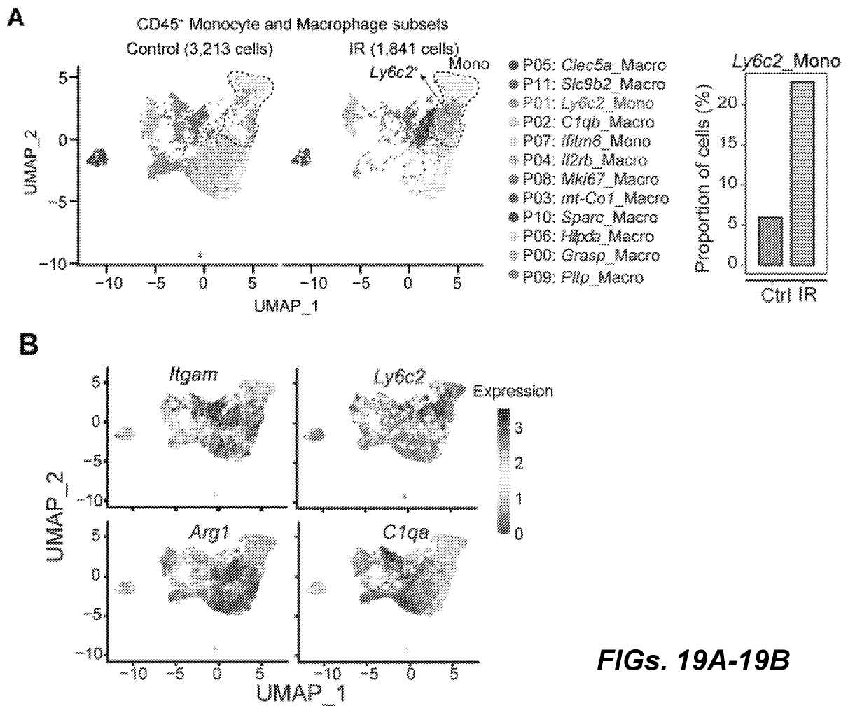

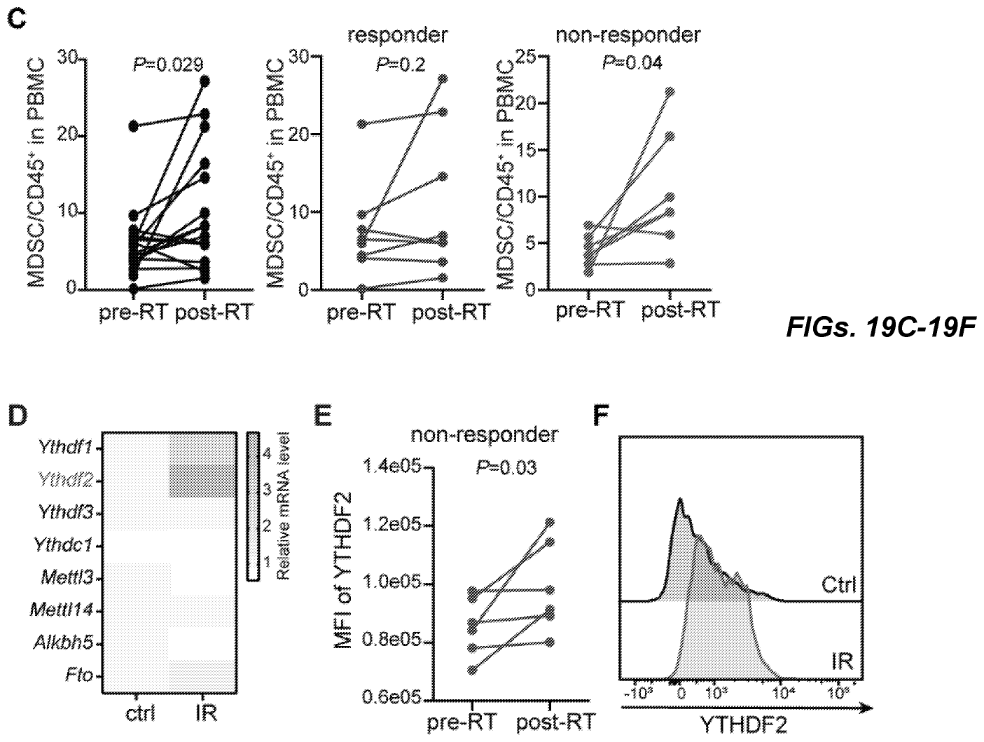

Focusing on the myeloid compartment, the inventors identified a monocytic MDSC cell subset (Ly6c2_Mono) in mice that dramatically increased in tumors post IR (FIGS. 19A, FIGS. 25E, and 25F), characterized by low expression of C1qa, a macrophage marker, and high expression of Ly6c2, CD11b, and Arg1 (FIGS. 19B and 25F). This observation is consistent with previous work identifying Ly6C⁺ monocytes as MDSCs with superior T cell suppressive function.²⁶ In an effort to translate this finding to humans, the inventors evaluated the levels of MDSCs in PBMCs from cancer patients enrolled in a clinical trial; patients were treated with radiotherapy followed by pembrolizumab (anti-PD-1) (NCT02608385).⁵⁶ MDSCs were significantly increased post RT compared with matched pre-RT levels (p=0.29) in PBMCs. Notably, this increase was significant in patients who progressed at distal sites (outside of the radiation treatment fields; non-responders) (p=0.04), but it was not significantly changed in patients who did not progress at distal sites (responders) (p=0.2) (FIG. 19C). Consistent results were observed in PBMCs from metastatic non-small-cell lung cancer patients enrolled in another clinical trial (the COSINR study, NCT0322315557) (FIG. 25G). In addition, the inventors analyzed datasets in the TCGA database with MDSCs gene signature (ARG1, CD14, CD44, CD40, S100A8, SELPLG, STAT6, TFRC, TGFB2, STAT3, CD274, ITGA3, SLA, and KDR;⁵⁸) to investigate whether MDSC-associated genes are increased in clinical samples post RT and whether the increase correlates with poor outcome. A low MDSC signature was significantly associated with prolonged patient survival in a low-grade glioma cohort with RT (p=0.01) and in a glioblastoma cohort with RT (p=0.49) (FIG. 25H). These data indicate that radiation-induced MDSCs may be associated with worse clinical outcome in patients who receive local radiotherapy.

In addition to increased numbers of immunosuppressive MDSCs post RT. the inventors also investigated the impact of IR on epitranscriptomic modifications driven by RNA m⁶A methylation, which the inventors have recently shown also modulates host antitumor immunity.⁵⁰ The inventors observed that the expression of YTHDF2, a m⁶A reader protein, was dramatically elevated in MDSCs of irradiated vs. untreated tumors (FIG. 19D). In the PBMCs of patients with distal tumor progression, the YTHDF2 level in MDSCs was significantly increased post RT compared with matched pre-RT samples/values (p=0.03. FIG. 19E). Consistently, YTHDF2 protein level was markedly induced in MC38 tumor-infiltrating MDSCs after IR treatment (FIG. 19F) but not in other infiltrating immune cells (FIG. 25I). The inventors next interrogated the temporal response of YTHDF2 following IR and observed that YTHDF2 was markedly elevated in a time-dependent manner (FIG. 25J). IR also elicited a direct effect on YTHDF2 expression, as evidenced by upregulated YTHDF2 in bone marrow-derived CD11b⁺Ly6C⁺ cells treated with IR in a time- and dose-dependent manner (FIG. 25K). These results demonstrate that IR induces YTHDF2 expression in MDSCs in both clinical and preclinical settings.

### Example 12: Ythdf2 Deficiency in Myeloid Cells Improves Response to Radiotherapy

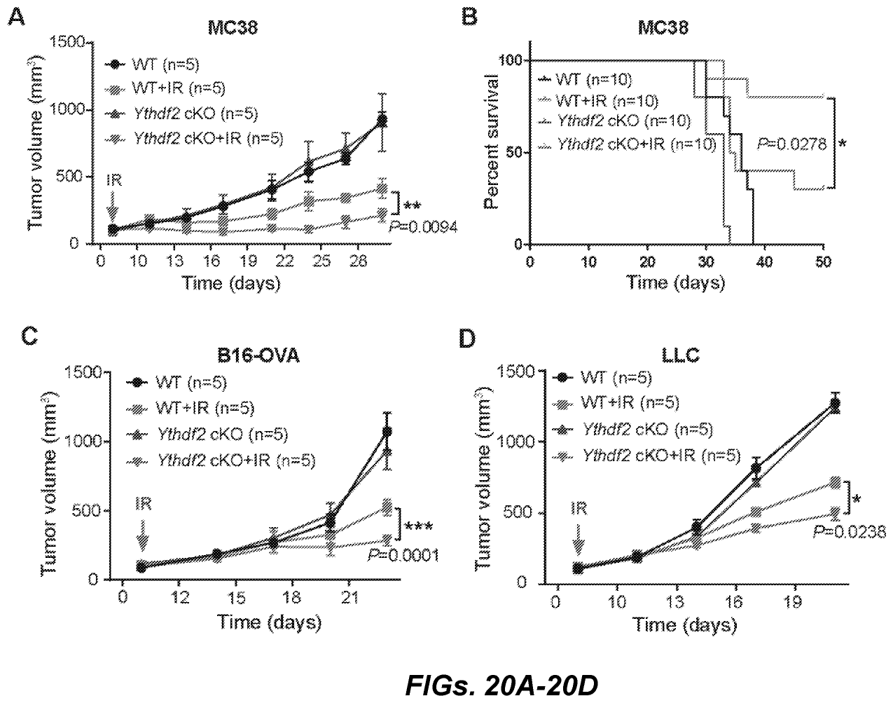

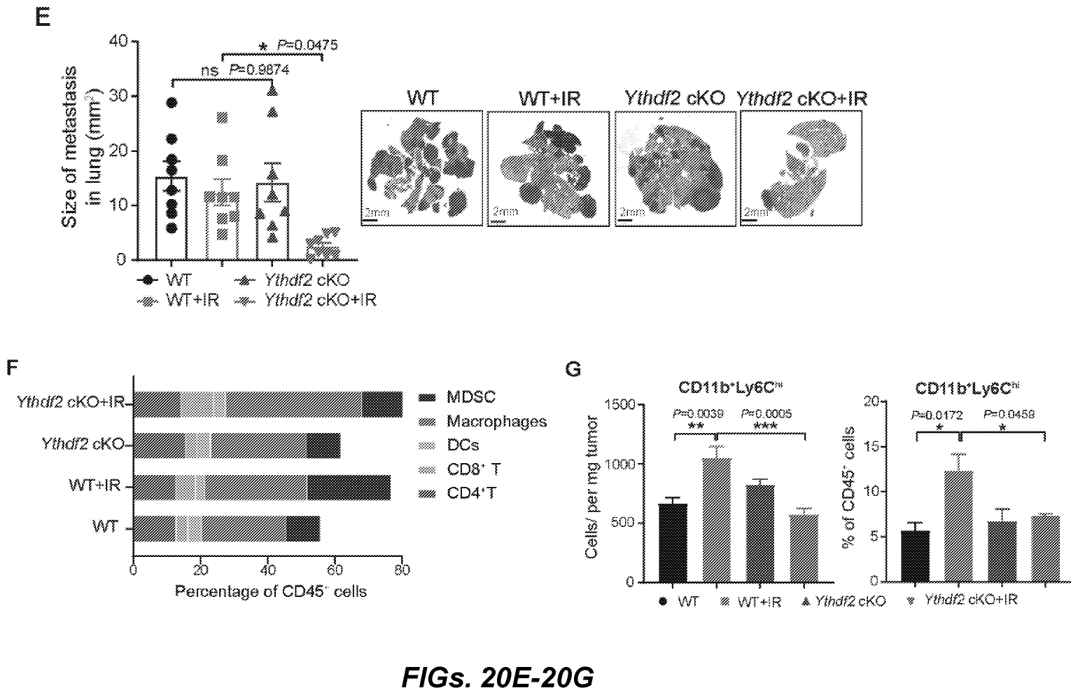

The inventors reasoned that increased YTHDF2 in myeloid cells could alter the response to radiotherapy. To test this, the inventors employed Lyz^{Cre+}; Ythdf2^{fl/fl} conditional knockout mice (hereafter Ythdf2-cKO) and Ythdf2^{fl/fl} (hereafter WT) in the C57BL/6J genetic background for tumor growth experiments. In the syngeneic murine colon carcinoma (MC38) model, primary tumor growth in WT and Ythdf2-cKO mice was similar (FIG. 20A). By contrast, local irradiation treatment resulted in a pronounced inhibition of tumor growth in Ythdf2-cKO mice compared with WT mice, assessed by both tumor volume and animal survival (FIGS. 20A and 20B). The inventors also irradiated melanoma and Lewis lung carcinoma (LLC) tumors in both WT and Ythdf2-cKO mice and observed a similar phenotype (FIGS. 20C and 20D). Intriguingly, in the LLC spontaneous lung metastasis model, a reduced metastatic burden was observed in lungs of Ythdf2-cKO mice that received IR compared with WT mice that received IR (FIG. 20E). Taken together, these data indicate that deleting Ythdf2 in myeloid cells enhanced the efficacy of radiotherapy through an increase in both local and distal metastasis control.

To assess whether distinct myeloid cell subsets are implicated in tumor progression and antitumor immunity following IR, the inventors first characterized the effects of Ythdf2 deletion in the TME after IR by profiling the tumor-infiltrating immune cells in MC38 tumors using flow cytometry. MDSCs and CD8⁺ T cells exhibited significant changes (FIG. 20F). In WT mice, both the absolute number and percentage of CD11b⁺Ly6C^{hi} cells (monocytic MDSCs) increased in irradiated tumors compared with controls. In Ythdf2-cKO mice, the level of MDSCs did not increase in irradiated tumors and remained similar to the level in non-irradiated tumors from WT mice (FIG. 20G). The level of MDSCs in irradiated tumors from Ythdf2-cKO mice was significantly decreased both in absolute number (p=0.0005) and percentage (p=00.0459; FIG. 20G) compared with irradiated tumors in WT mice. The results demonstrate that Ythdf2 deletion in myeloid cells led to a reduction of tumor-infiltrating MDSCs.

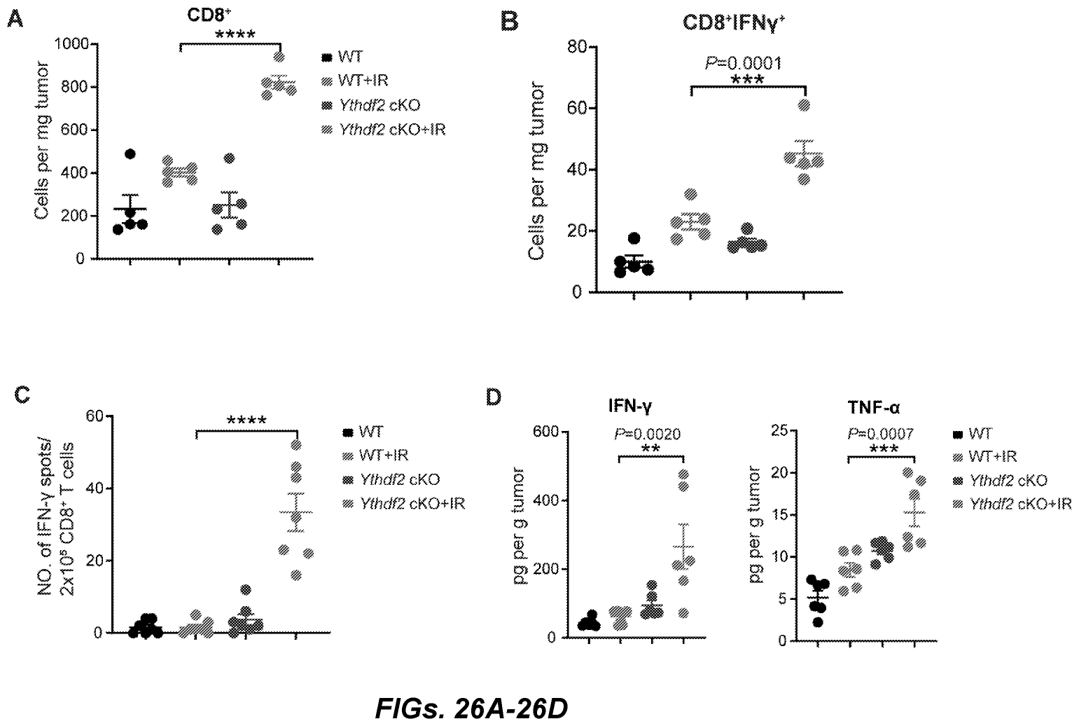

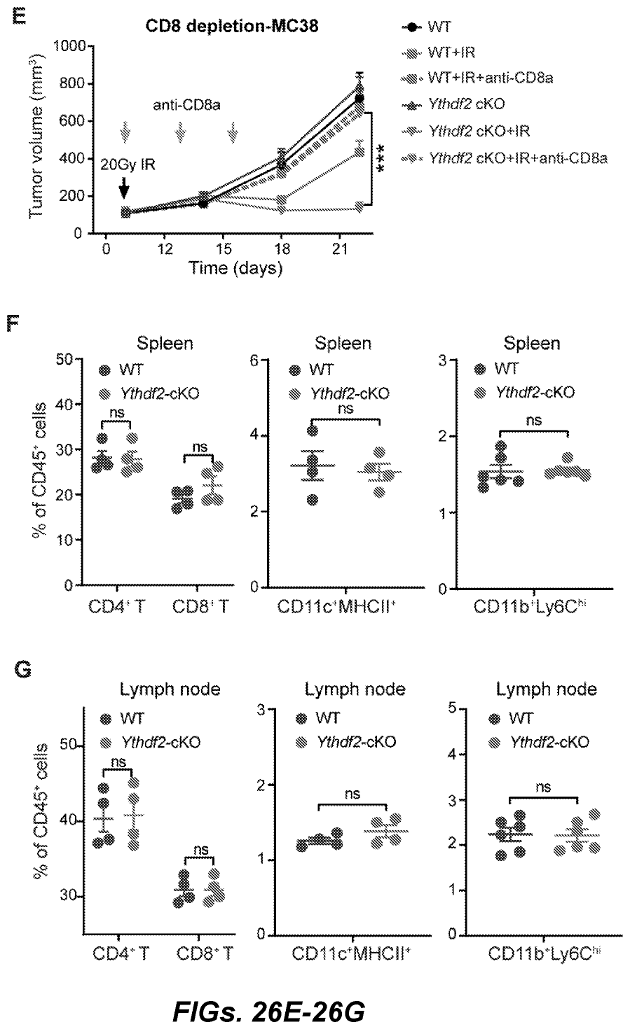

To examine whether specific deletion of myeloid Ythdf2 results in improved immune function of T cells, the inventors first measured the numbers of T cells and observed that both total CD8⁺ T cells and cytotoxic CD8⁺ T cells (IFNg⁺CD8⁺) were significantly increased in irradiated tumors in Ythdf2-cKO mice compared with those of irradiated tumors from WT mice (FIGS. 26A and 26B). ELISPOT assays measuring the IFN-g secreting capacity of CD8⁺ T cells sorted from the irradiated tumors in Ythdf2-cKO mice consistently showed a significant increase of IFN-g production (FIG. 26C). The inventors also found increased levels of both IFN-g and tumor necrosis factor (TNF)-a (FIG. 26D), representing enhanced cytotoxic function. The antibody-mediated depletion of CD8⁺ T cells completely abrogated the antitumor efficacy of IR in Ythdf2-cKO mice (FIG. 26E). Considering that conditional knockout of Ythdf2 did not affect the development of T cells in naive mice (FIGS. 26F and 26G), the findings indicate that the CD8⁺ T cells are essential for enhanced IR-induced tumor control likely due to decreased MDSCs in Ythdf2-cKO mice.

### Example 13: IR Reshapes the Composition of MDSC Populations in Blood and Tumors

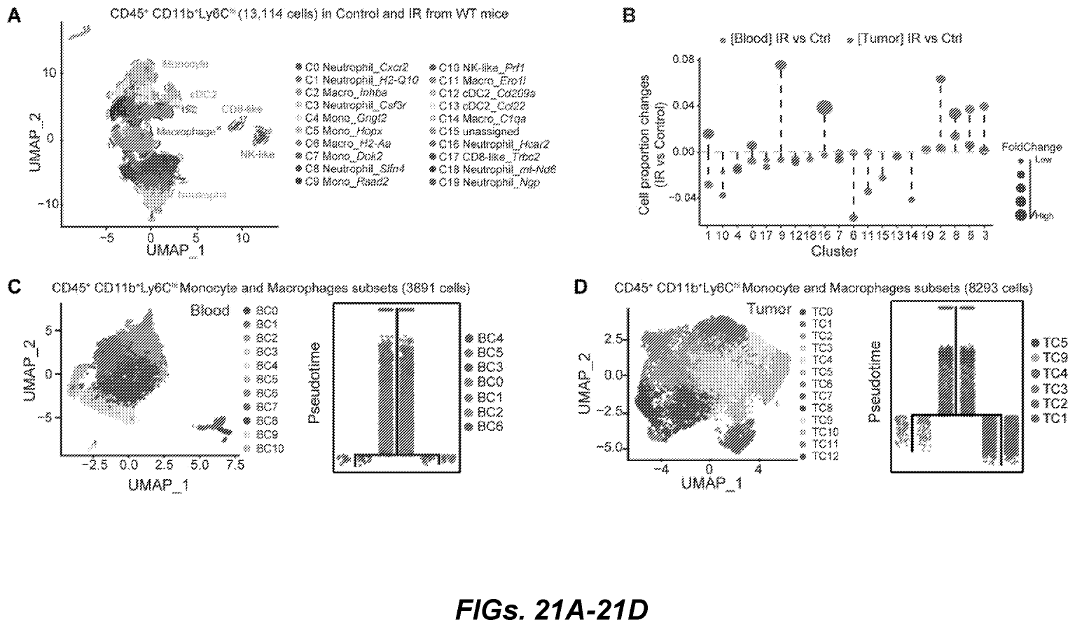

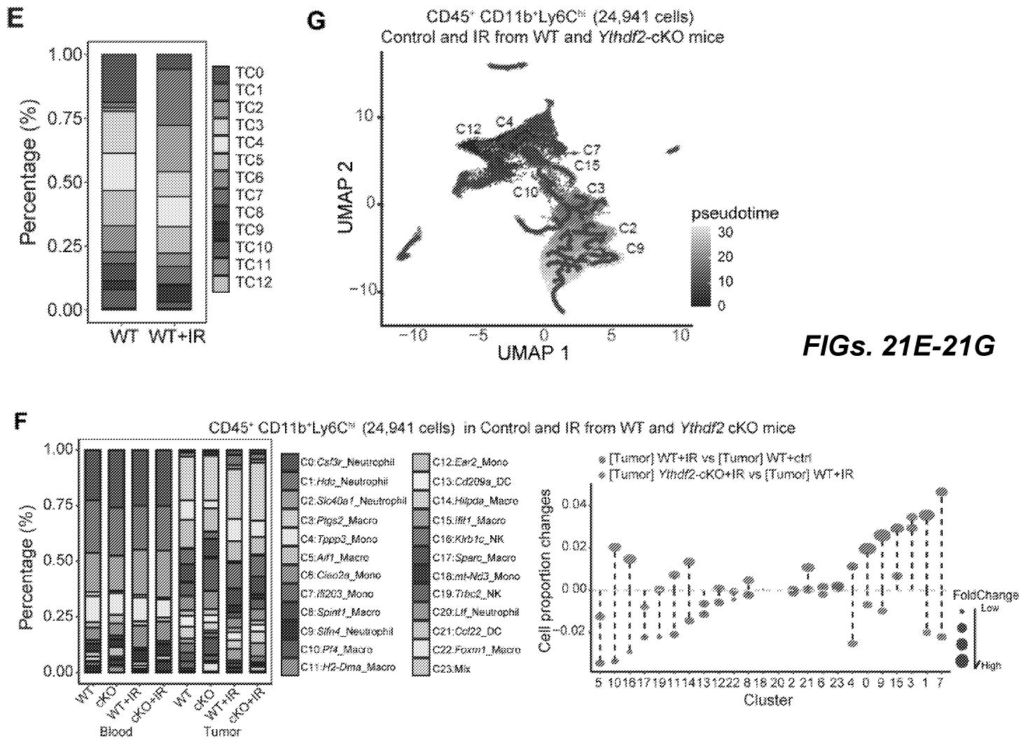

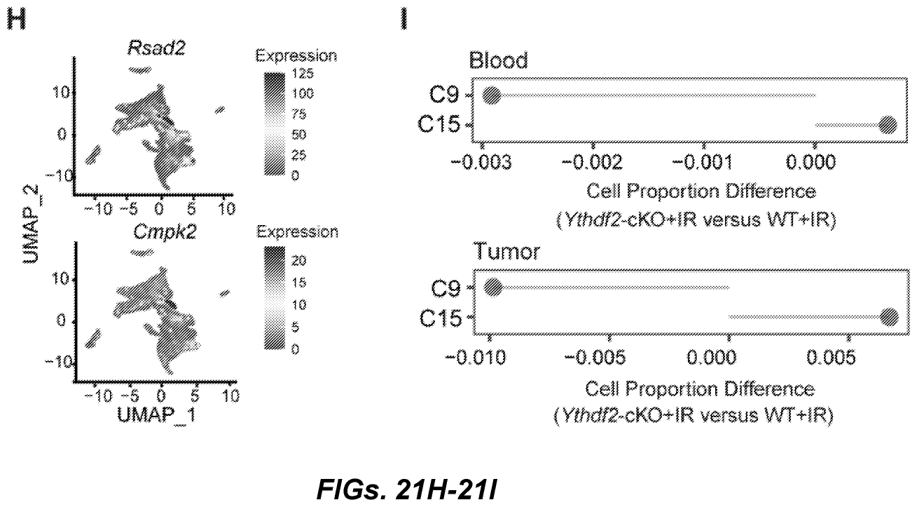

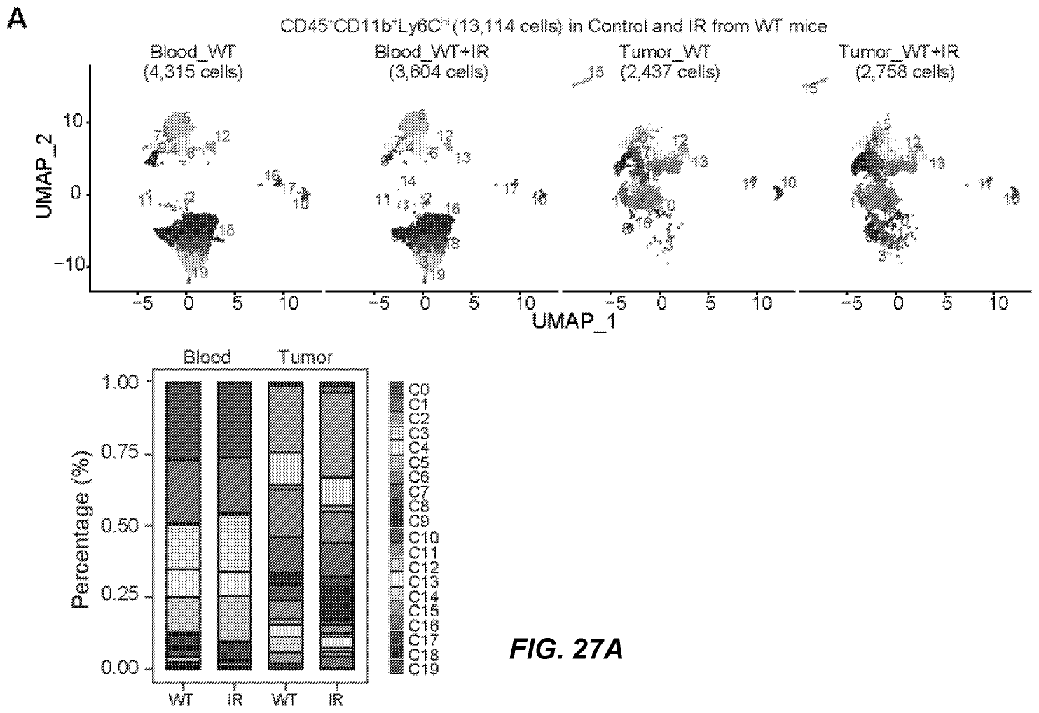

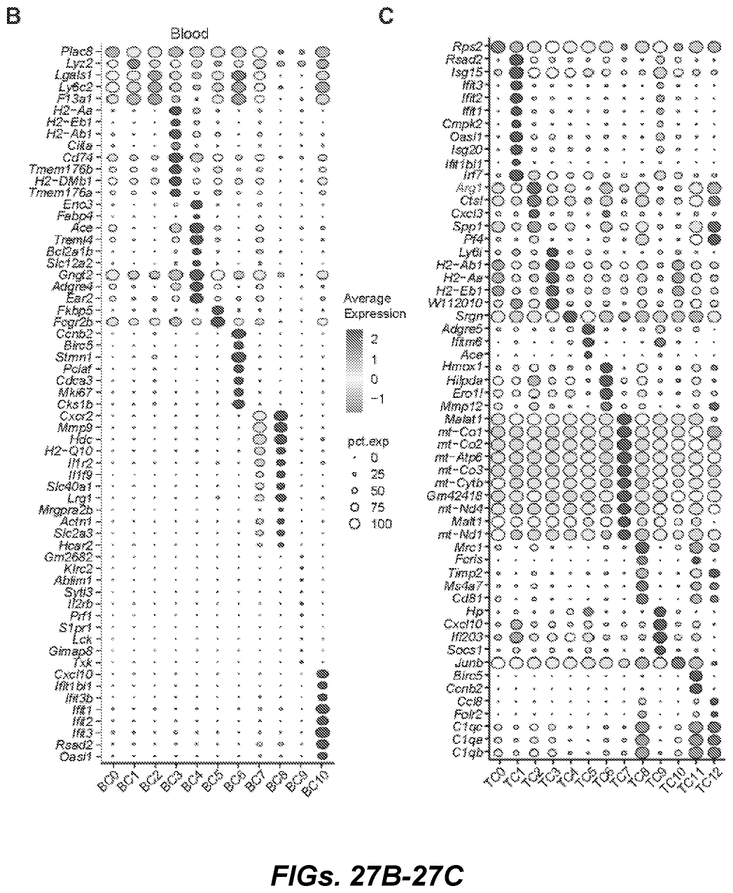

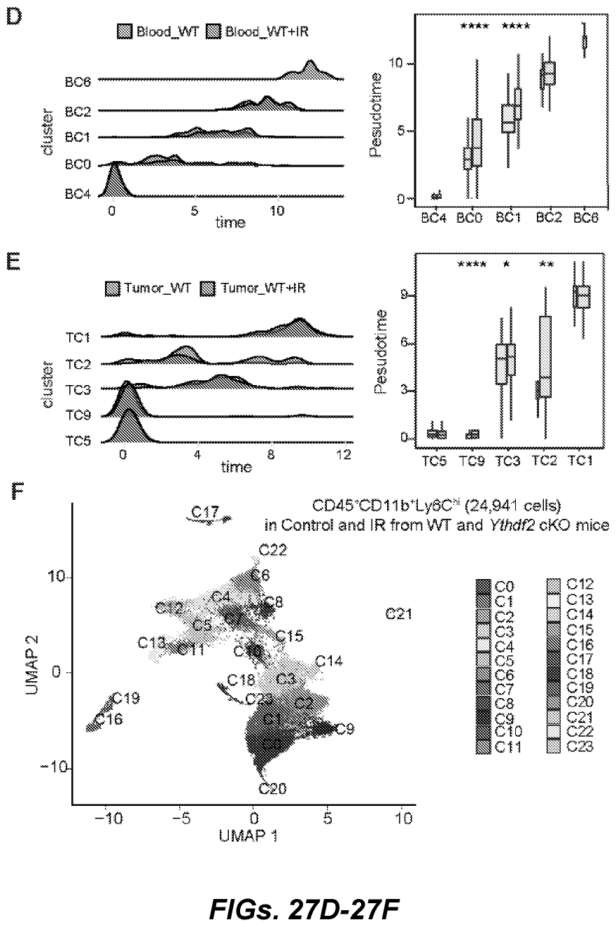

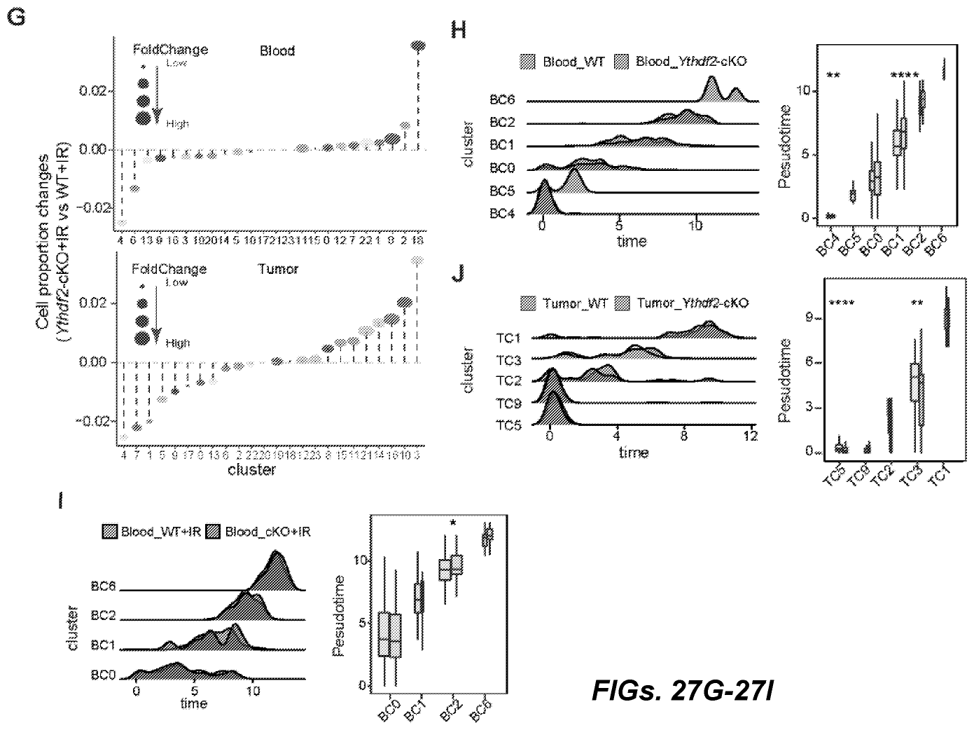

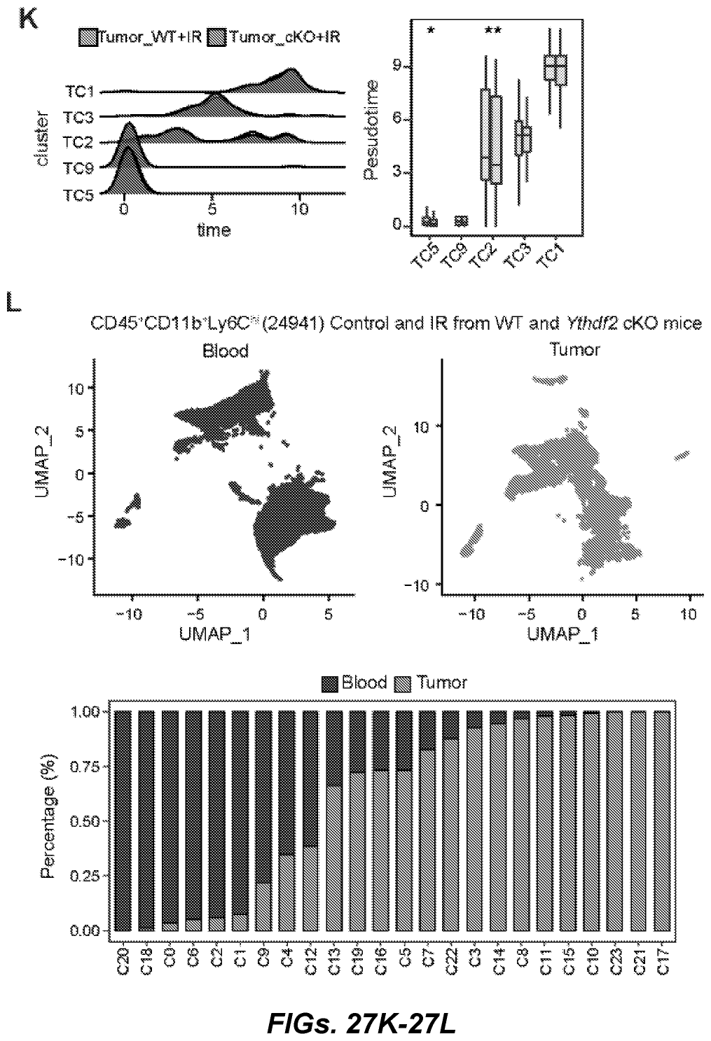

The inventors aimed to further delineate the effects of YTHDF2 on MDSCs in the context of IR. The inventors first investigated the state of monocytic MDSCs (mMDSCs), which are immature myeloid cells, by performing scRNA-seq using CD45⁺CD11b⁺Ly6C^{hi} cells (mMDSCs) isolated from blood and MC38 tumors of irradiated mice. To dissect mMDSC heterogeneity, the inventors applied unbiased clustering algorithms and identified 19 distinct cell populations belonging to four broad cell types: monocytes, macrophages, DCs, and neutrophils (FIGS. 21A and 27A). In blood, IR consistently modulates mMDSC development. For example, C3 (Neutrophil-Csf3r) and C5 (Mono-Hopx) (labeled blue) were increased in blood after IR; C9 (Mono-Rsad2) and C2 (Macro-Inhba) (labeled red) were increased in tumors after IR (FIG. 21B). Local tumor IR modulates mMDSC development intratumorally and systemically, demonstrating that local IR alters systemic immune responses.

To infer the potential differentiation trajectories, the inventors compiled monocyte and macrophage subsets of mMDSCs derived from blood along a pseudotime axis and observed that the differentiation occurs on a tightly organized trajectory, starting from BC4 cluster, through BC0, BC1, BC2, and ending with the BC6 cluster (FIG. 21C). BC6 exhibits cluster-specific expression of Ccnb2, Birc5, Stmn1, Pclaf, Cdca3, Mki67, and Cks1b (FIG. 27B), indicative of highly proliferative activity. In tumors, the inventors also zoomed in on monocyte and macrophage subsets and found that cluster TC5 gradually develops into TC9, then TC1 or into TC2 and TC3 (FIG. 21D). Notably, the immunosuppressive gene Arg1 was highly expressed in 10 out of 13 clusters, indicative of a suppressive phenotype (FIG. 27C). The proportion of TC1 and TC2 cells, annotated by gene expression signature as inflammatory and suppressive mMDSC population, respectively, were increased following IR compared with non-IR condition (FIG. 21E). IR also resulted in the decrease of several populations in tumor mMDSCs, such as TC0, annotated with high ribosomal activity, and TC3, which exhibits the expression of MHC class-associated genes (H2-Ab1, H2-Aa, H2-Aa, and H2-Eb1), suggesting that it could be classified as TAM with cross-presentation activity. To investigate the role of IR in the differentiation of mMDSCs in blood, the inventors next quantified the pseudotime of each cell subset in irradiated and unirradiated mice. The inventors found that BC0 and BC1 from irradiated mice showed increased pseudotime compared with unirradiated controls (FIG. 27D). The inventors also observed significantly increased pseudotime of TC9. TC2, and TC3 from irradiated mice vs. unirradiated controls (FIG. 27E). Together, the findings reveal that tumor-local IR remodeled the landscape of mMDSC populations, possibly through accelerating mMDSC differentiation, and triggered a suppressive tumor microenvironment.

### Example 14: YTHDF2 Affects mMDSC Differentiation in the Context of IR

To assess the relationship between YTHDF2 inhibition and mMDSC differentiation and to obtain a full picture of the population change, the inventors queried scRNA-seq data of mMDSCs (all CD45⁺CD11b⁺Ly6C^{hi} cells) from Ythdf2-cKO mice. Ythdf2 knockout led to changes in the proportion of distinct mMDSC-derived subsets in both blood and tumor in irradiated and unirradiated controls, compared with WT mice (FIGS. 21F and 27F-27G). First, the inventors conducted the trajectory analysis of mMDSC-derived subsets in blood and observed that, in unirradiated mice. BC4 and BC1 showed increased pseudotime in Ythdf2-cKO mice compared with WT (FIG. 27H). In irradiated mice. BC2 showed increased pseudotime in Ythdf2-cKO mice compared with WT (FIG. 27I). Second, the inventors conducted the trajectory analysis of mMDSC-derived subsets in tumor and found that TC5 and TC3 showed a significantly decreased pseudotime in Ythdf2-cKO compared with the WT (FIG. 27J). TC5 and TC2 showed decreased pseudotime in Ythdf2-cKO+IR compared with the WT+IR (FIG. 27K). Strikingly, the pattern of cell population changes in “Ythdf2-cKO+IR vs. WT+IR” is largely opposite to that in “WT+IR vs. WT” (FIG. 21F), suggesting YTHDF2 plays a key role in MDSC differentiation in response to IR. By establishing a single-cell atlas, the inventors demonstrated that Ythdf2 knockout alters the mMDSC differentiation and the effects are amplified by IR.

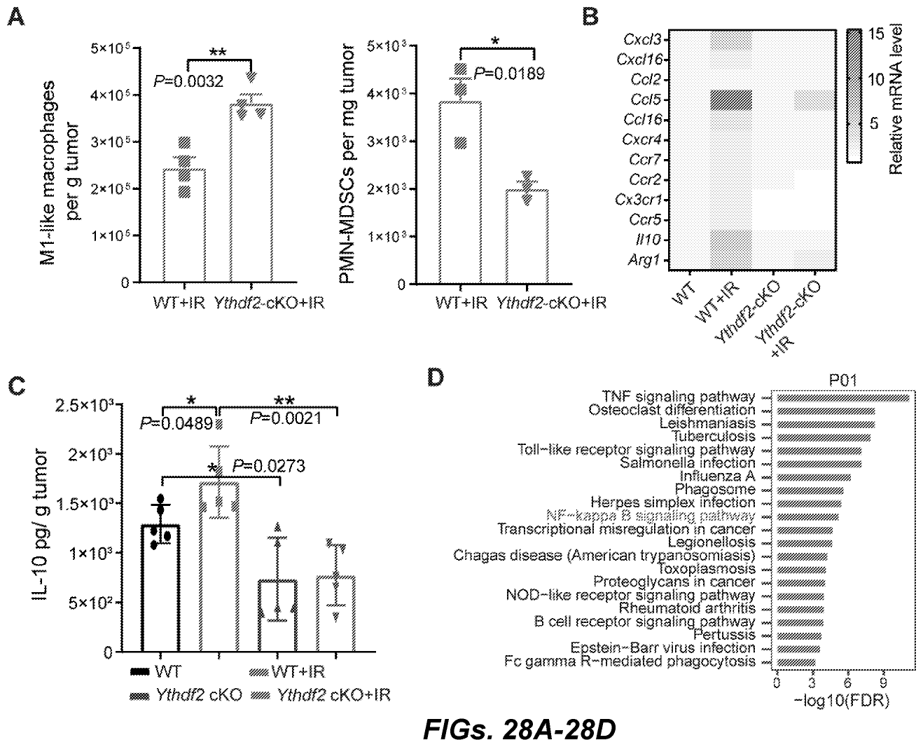

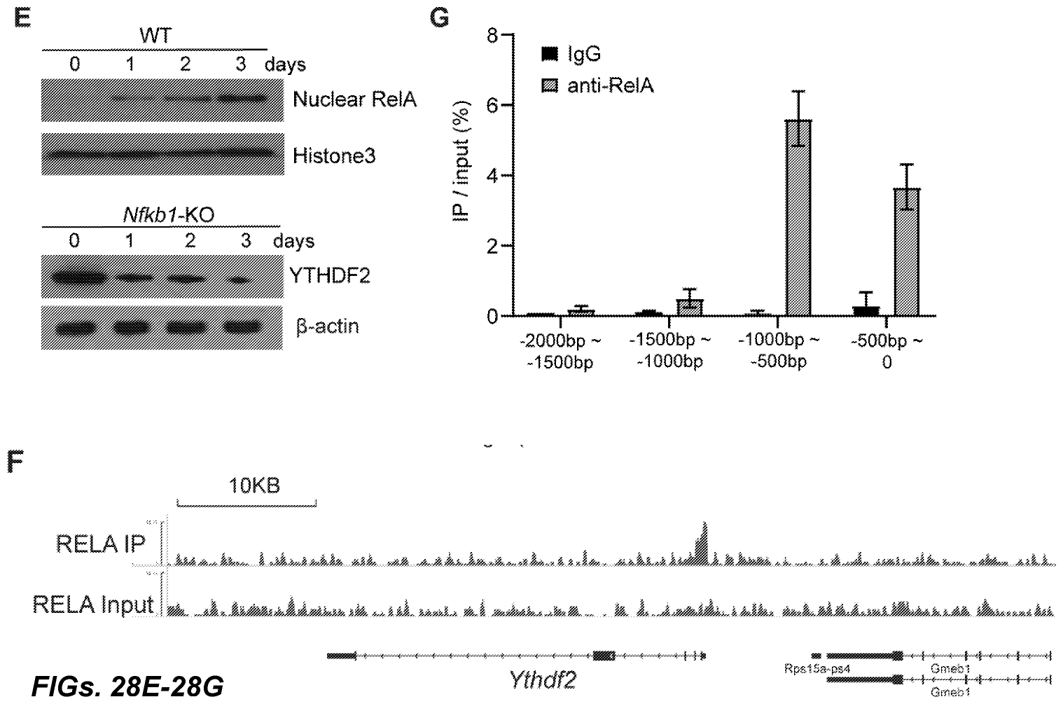

MDSCs migrate from blood to tumor in response to radiotherapy. To depict a continuous picture of mMDSC differentiation, the inventors conducted the trajectory analysis in mMDSCs combining all samples (blood and tumor from WT or Ythdf2-cKO+/−IR treatment) (FIGS. 21G and 27F). The inventors identified C12 as monocyte precursor on the basis of abundant expression of Ear2.⁵⁹ The C12 population evolves into C4, and then C7, followed by branching into two separate paths: 1) C15 and 2) from C10, C3, C2, to C9 (FIG. 21G). Among them, C12 and C4 mainly reside in blood, and C7 mainly associates with tumor (FIG. 27L). C15 is classified as an M1-like macrophage, characterized by high levels of Rsad2 and Cmpk2 (FIG. 21H). C3, C2, and C9 were characterized as polymorphonuclear (PMN)-MDSCs-like cells, consistent with a concept that mMDSCs can differentiate into PMN-MDSCs.⁶⁰) The percentage of C15 (M1-like cells) significantly increased, while C9 (PMN-MDSC-like cells) significantly decreased in IR treated Ythdf2-cKO mice versus IR treated WT mice (FIG. 21I), as further confirmed by flow cytometry analysis (FIG. 28A); this data mirrors the tumor growth phenotypes. These modifications of mMDSC-derived clusters resulted in reprogramming of the host immune microenvironment locally and systemically in favor of enhanced anti-tumor immunity in Ythdf2-cKO mice in response to IR.

### Example 15: YTHDF2 Controls MDSC Migration and Suppressive Function in the Context of IR

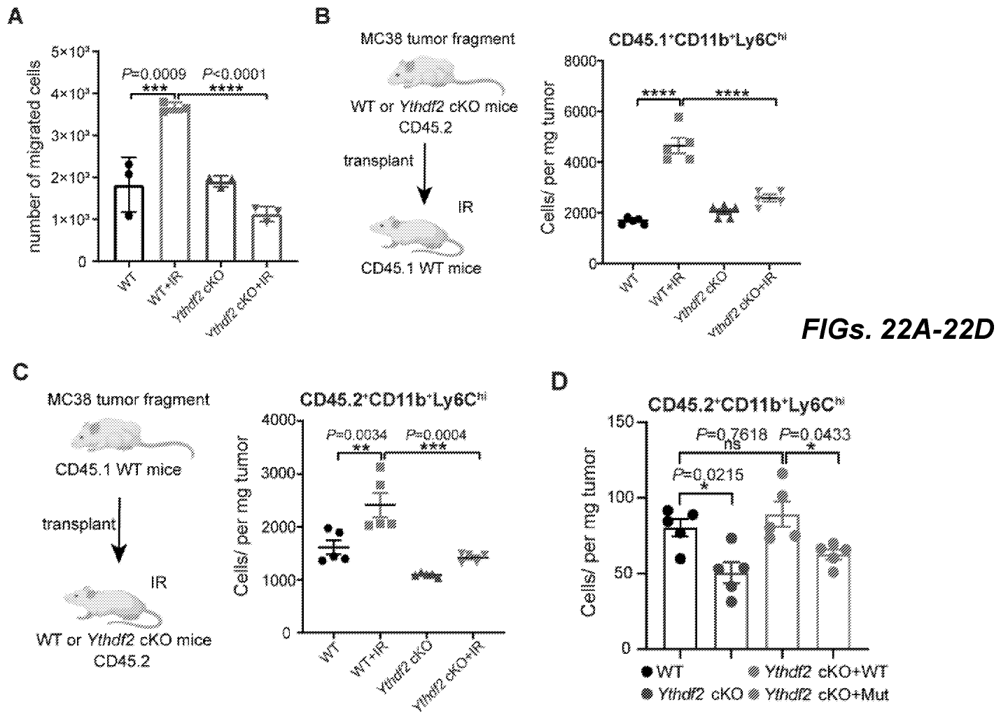

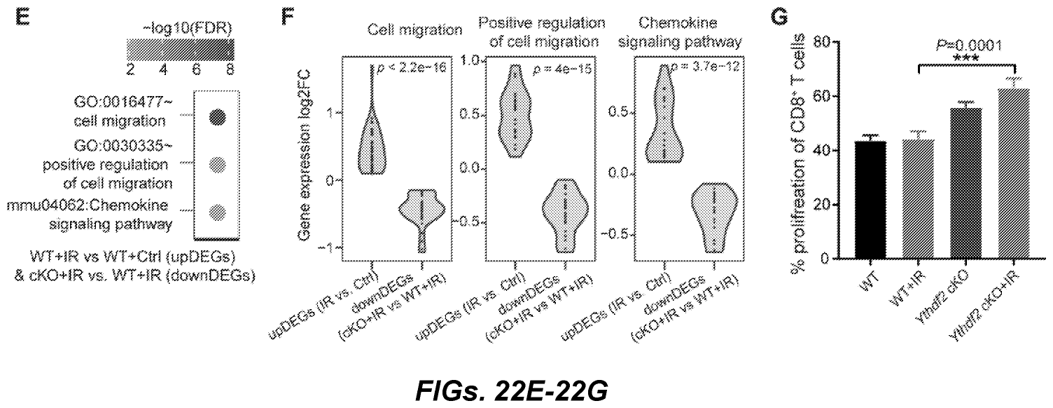

Having demonstrated that YTHDF2 affects MDSC differentiation in the context of IR, the inventors next investigated the role of YTHDF2 in IR-induced MDSC migration. Ythdf2 deletion impaired the migratory capacity of MDSCs as evidenced by a migration assay in which MDSCs in irradiated tumors of Ythdf2-cKO mice showed a significantly lower migration compared with that of WT mice (FIG. 22A). To further verify this. MC38 tumor fragments from WT or Ythdf2-cKO mice (both of which are CD45.2), which contain pre-existing MDSCs, were harvested and inoculated into CD45.1 WT mice. The CD45.1 mice were treated with IR. Three days after IR, there was no significant increase of tumor-infiltrating CD45.1⁺CD11b⁺Ly6C^{hi} (mMDSC) cells post-IR in tumors derived from Ythdf2-cKO mice, whereas a significant increase in this population was observed in tumors derived from WT mice post-IR (FIG. 22B). Further, the inventors observed consistent changes in chemokine expression in the infiltrating MDSCs (FIG. 28B). These results suggest that loss of Ythdf2 in myeloid cells abrogated IR-induced enhanced chemokine production to attract further infiltration of MDSCs. The inventors also inoculated MC38 tumor fragments grown in CD45.1-WT mice into WT or Ythdf2-cKO mice (CD45.2) and found that IR failed to induce the accumulation of tumor-infiltrating CD45.2⁺ MDSCs in Ythdf2-cKO mice (FIG. 22C), suggesting that Ythdf2-cKO MDSCs were less effective in trafficking to the tumor following IR, possibly due to the decrease of certain chemokine receptor expression (FIG. 28B). To further demonstrate that the effect of YTHDF2 on MDSC migration is dependent on its m⁶A binding, the inventors force expressed YTHDF2 (Ythdf2-WT) and m⁶A-binding-site-mutated YTHDF2 (Ythdf2-Mut) in Ythdf2-deficient BM-MDSCs (CD45.2) and adoptively transferred these cells into MC38 tumor-bearing CD45.1 mice followed by IR treatment. Three days post-IR, the number of newly-infiltrated CD45.2 MDSCs in tumors were analyzed. Compared to transferred WT-MDSCs, Ythdf2-cKO-MDSCs elicited significantly lower migration capacities post-IR (P=0.0215). YTHDF2-WT overexpressing Ythdf2-cKO-MDSCs rescued the migration (Ythdf2-cKO+WT vs. WT, P=0.7618), whereas YTHDF2-Mut overexpression could not rescue migration (Ythdf2-cKO+WT vs. Ythdf2-cKO+Mut, P=0.0433) (FIG. 22D). Taken together, the results indicate that deletion of Ythdf2 in the myeloid compartment leads to defects in both migration capacity and chemoattraction of MDSCs following IR, and the phenotype requires the m⁶A binding capacity of YTHDF2.

For validation of these findings, the inventors performed mRNA-seq using MC38 tumor-infiltrating CD11b⁺ myeloid cells in WT and Ythdf2-cKO mice+/−IR. As expected, the GO enrichment analysis indicated that three pathways, including those affecting cell migration, chemokine signaling, and positive regulation of cell migration, were up-regulated in WT+IR versus WT+ctrl, and were down-regulated in Ythdf2-cKO+IR versus WT+IR (FIGS. 22E-22F). These findings support the observation of MDSC migration phenotypes in vivo.

In terms of suppressive function, the inventors observed that, in the context of IR, tumor-infiltrating Ythdf2-cKO MDSCs exhibited attenuated suppressive function during co-culture with activated naïve CD8⁺ T cells, compared with WT MDSCs (FIG. 22G). In pursuing this further, the inventors investigated the expression levels of proteins produced by MDSCs that mediate immune suppression. IR significantly induced IL-10 production and Arg1 expression in tumors in WT mice compared with Ythdf2-cKO mice (FIG. 28B-28C). Taken together, these results reveal that Ythdf2 knockout impairs both MDSC migration and suppressive functions, which may be critical to the enhanced antitumor effect of IR observed in Ythdf2-cKO.

### Example 16: NF-κB/RELA Mediates Radiation-Induced YTHDF2 Expression in MDSCs

The inventors sought to investigate the potential mechanisms involved in radiation induction of YTHDF2 in MDSCs. First, the inventors performed functional enrichment analysis with genes differentially expressed in “monocytic MDSC_Ly6c2” (P01 population. FIG. 19A) following IR treatment, and found that genes of the NF-kappa B (NF-κB) signaling pathway are enriched in this population post-IR (FIG. 28D). These data are consistent with previous findings that IR activates NF-κB.⁶¹⁻⁶³ Further investigation of the relationship between NF-κB and YTHDF2 revealed that the level of nuclear RELA is increased after IR (FIG. 28E). YTHDF2 expression was not induced by IR in MDSCs deficient of Nfkb1 (FIG. 28E), demonstrating that NFKB1 is required for IR induction of YTHDF2. NFKB1 is an important component in NF-κB signaling by forming a RELA/NFKB1 heterodimer required for RELA nuclear translocation.⁶⁴ The inventors next analyzed a public dataset of RELA chromatin immunoprecipitation sequencing (ChIP-seq) conducted in mouse bone marrow-derived macrophages and found the predicted direct binding between RELA and the Ythdf2 promoter region (FIG. 22F). To verify this finding, the inventors performed ChIP coupled with quantitative PCR (ChIP-qPCR) analysis using bone marrow-derived CD11b⁺Ly6C⁺ cells. The results revealed that RELA indeed directly binds to the Ythdf2 promoter region (˜1.0-2.0 kb proximal to the transcription start site) (FIG. 22G). Collectively, these findings indicate that IR upregulates YTHDF2 expression via the NF-κB/RELA signaling pathway.

### Example 17: IR-Induced YTHDF2 Enhances NF-kB Signaling by Promoting m6A-Modified RNA Degradation

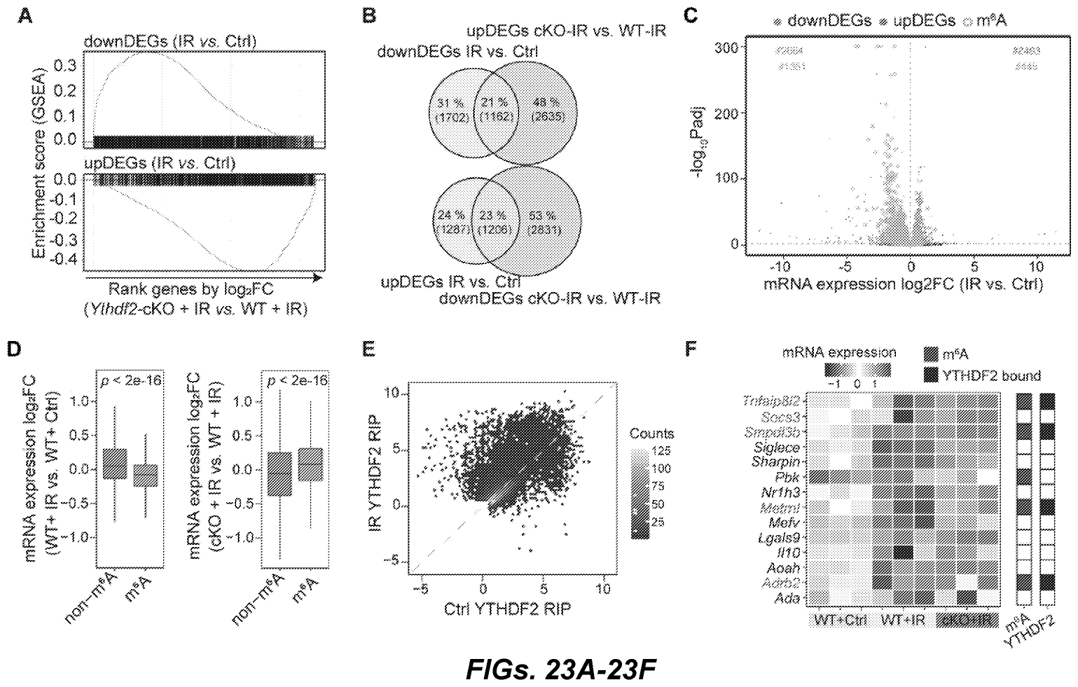

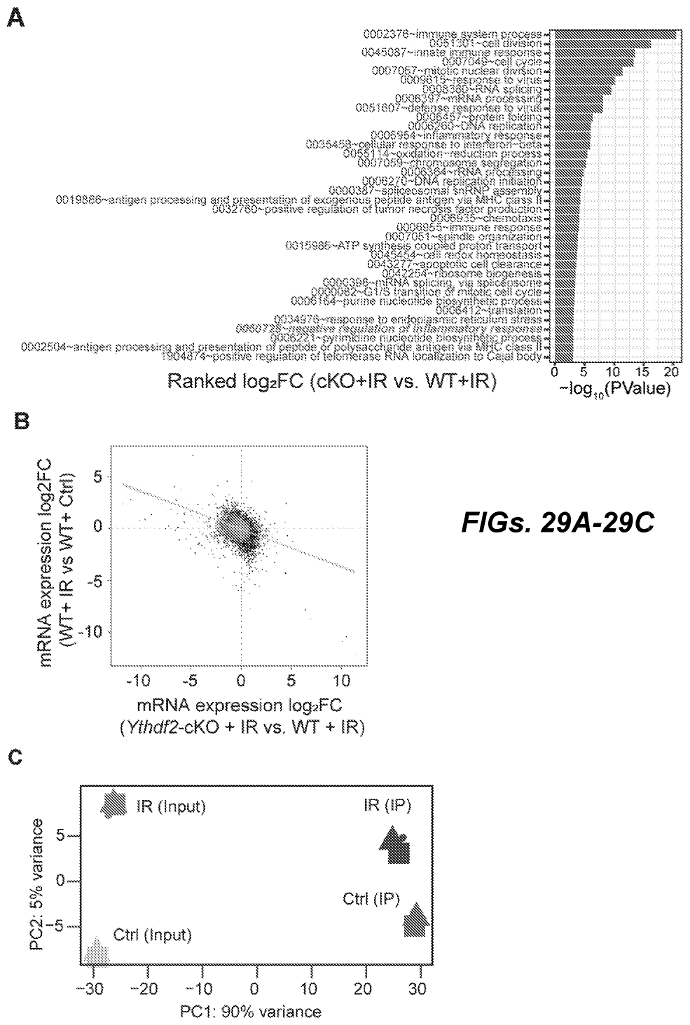

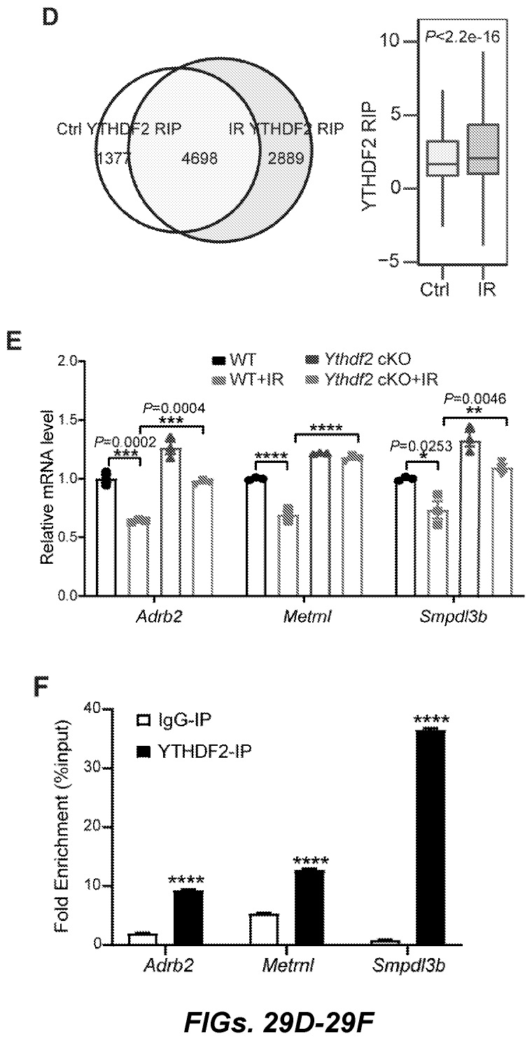

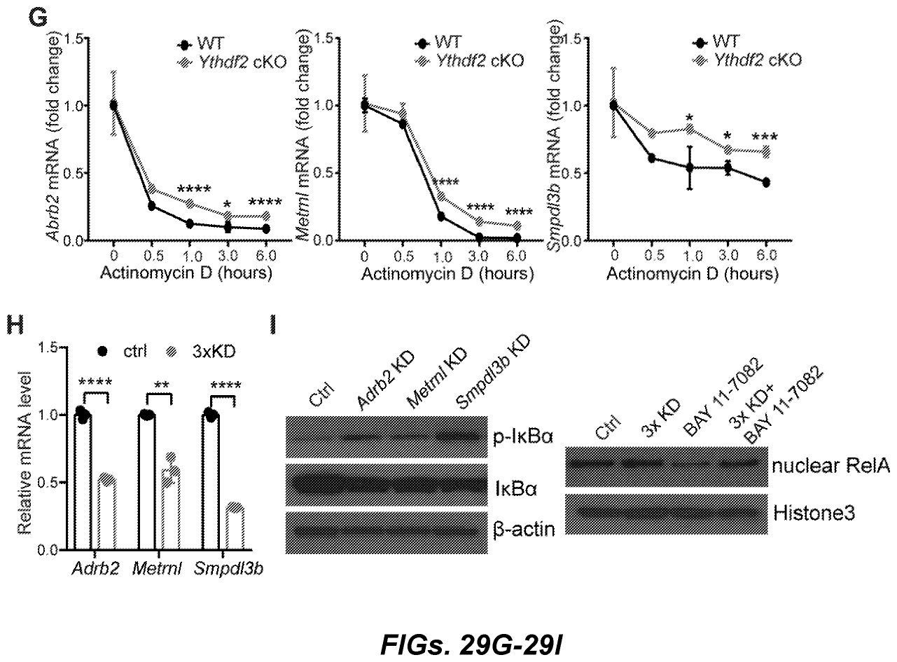

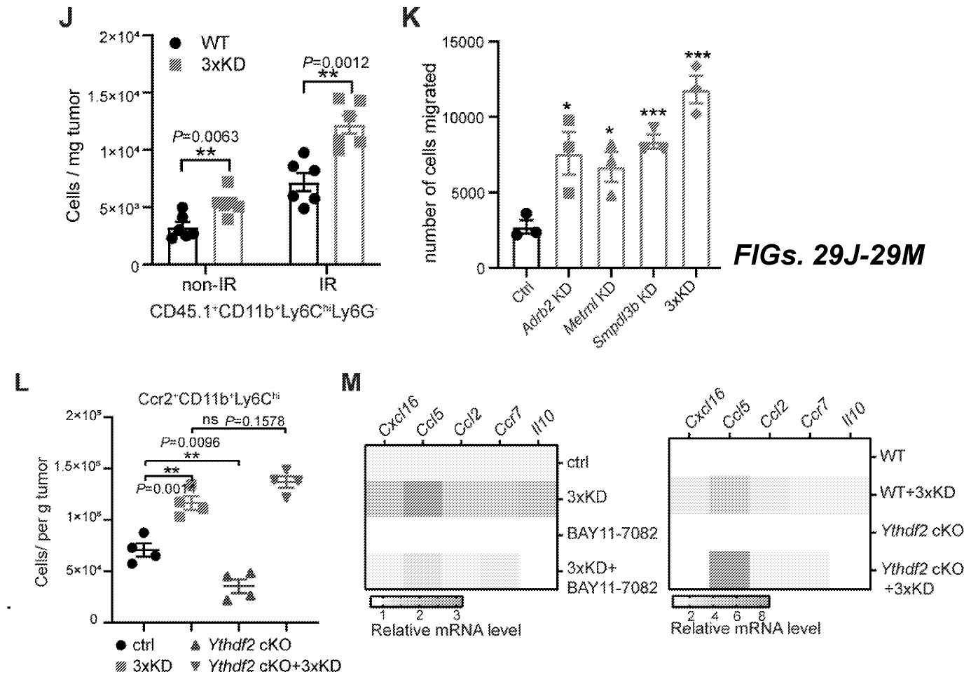

To investigate the molecular mechanisms of YTHDF2 function in MDSCs in the context of IR, the inventors reanalyzed the mRNA-seq of MC38 tumor-infiltrating CD11b⁺ myeloid cells in Ythdf2-cKO mice with IR or unirradiated controls. The inventors analyzed the gene expression profiles and found that knockout of Ythdf2 abolished the transcriptional changes induced by IR alone (FIG. 23A). The inventors focused on the differentially expressed genes, comparing IR versus non-IR in WT mice and IR versus non-IR in Ythdf2-cKO mice (FIG. 23B) to perform gene enrichment analysis. The inventors found an enrichment of the “negative regulation of inflammatory response” pathway in Ythdf2-cKO+IR (FIG. 29A), containing five genes (Tnfaip8l2, Socs3, Smpdl3b, Metrnl and Adrb2) which have been reported as negative regulators for NF-κB signaling.⁶⁵⁻⁶⁹ which facilitates MDSC migration and chemokine/cytokine regulation.⁷⁰ Thus the data indicate that IR induces YTHDF2 via NF-κB, and the elevated YTHDF2 levels may in turn enhance NF-κB signaling in MDSCs, thus forming an IR-YTHDF2-NF-κB circuit.

To test this hypothesis, the inventors sought to characterize the downstream direct targets of YTHDF2 by performing N⁶-methyladenosine-sequencing RNA immunoprecipitation followed by high-throughput sequencing (MeRIP-seq) and RNA immunoprecipitation sequencing (RIP-seq). The inventors observed that a majority of m⁶A-marked genes were down-regulated after IR treatment (1351 down-regulated genes versus 448 up-regulated genes) (FIG. 23C) and Ythdf2 deletion reversed this suppression (FIGS. 23D and 29B). Furthermore, with YTHDF2 expression being elevated by IR, the number of YTHDF2-bound transcripts increased accordingly (FIGS. 23E and 29C-29D), suggesting that increased YTHDF2 binding post IR mediated the decrease of mRNA abundance.

Close investigation of the above-mentioned genes as negative regulators of NF-κB signaling revealed three genes (Adrb2, Metrnl, and Smpdl3b) are m⁶A marked and targeted by YTHDF2 (FIGS. 23F and 29E-29F). Additionally, their transcript half times were increased in Ythdf2-cKO (FIG. 29G), consistent with the known function of YTHDF2 in promoting mRNA degradation. The inventors hypothesized that, in the context of IR, Ythdf2 enhances NF-κB signaling through reducing the expression of Adrb2, Metrnl, or Smpdl3b. To test this, the inventors generated BM-MDSCs with all 3 genes knocked down (3×KD) (FIG. 29H) and conducted western blot assay for NF-κB signaling. The inventors observed increased levels of IκBα phosphorylation and nuclear localization of RELA in knockdown MDSCs (FIG. 29I). Moreover, IκBα phosphorylation inhibitor (BAY 11-7082)⁷¹ treatment prevented this increase (FIG. 29I). These results indicate that the m⁶A/YTHDF2 axis regulates NF-κB signaling in MDSCs by targeting negative regulators of the NF-κB pathway.

To assess whether the IR-YTHDF2-NF-κB axis contributes to MDSC migration or function, the inventors employed Ccr2-KO mice in which the radiation-induced MDSC infiltration into tumors is markedly decreased²⁶. The inventors adoptively transferred 3×KD-MDSCs into MC38 tumor-bearing Ccr2-KO mice and observed that compared with the transferred WT-MDSCs, 3×KD-MDSCs elicited significantly higher migration capacities post-IR (FIG. 29J). In vitro migration assays of 3×KD-MDSCs also showed similar results (FIG. 29K). To further confirm the involvement of YTHDF2 in radiation-induced MDSC infiltration, the inventors performed the aforementioned transfer experiment using 3×KD-Ythdf2-cKO-MDSCs. As expected, three days after IR, the level of infiltrating MDSCs (Ccr2⁺CD11b⁺Ly6C^{hi}) was restored to a level similar to 3×KD-MDSCs transfer (FIG. 29L). Functionally, the inventors observed consistent changes in the expression of genes associated with migration and function of MDSCs, including Ccl2, Ccl5, Cxcl16, Ccr7, and Il10 (FIG. 29M). Collectively, these data demonstrate that the enhanced migration and function of MDSCs induced by IR rely on the expression of YTHDF2, most likely via enhanced activation of the NF-κB pathway. Together with the previous finding that IR-mediated YTHDF2 induction relies upon NF-κB activation, the inventors propose that an YTHDF2-NF-κB positive feedback loop governs migration and suppression of MDSCs and tumor extrinsic radioresistance.

### Example 18: Pharmacological Inhibition of YTHDF2 Enhances Responses to Radiotherapy and Immunotherapy

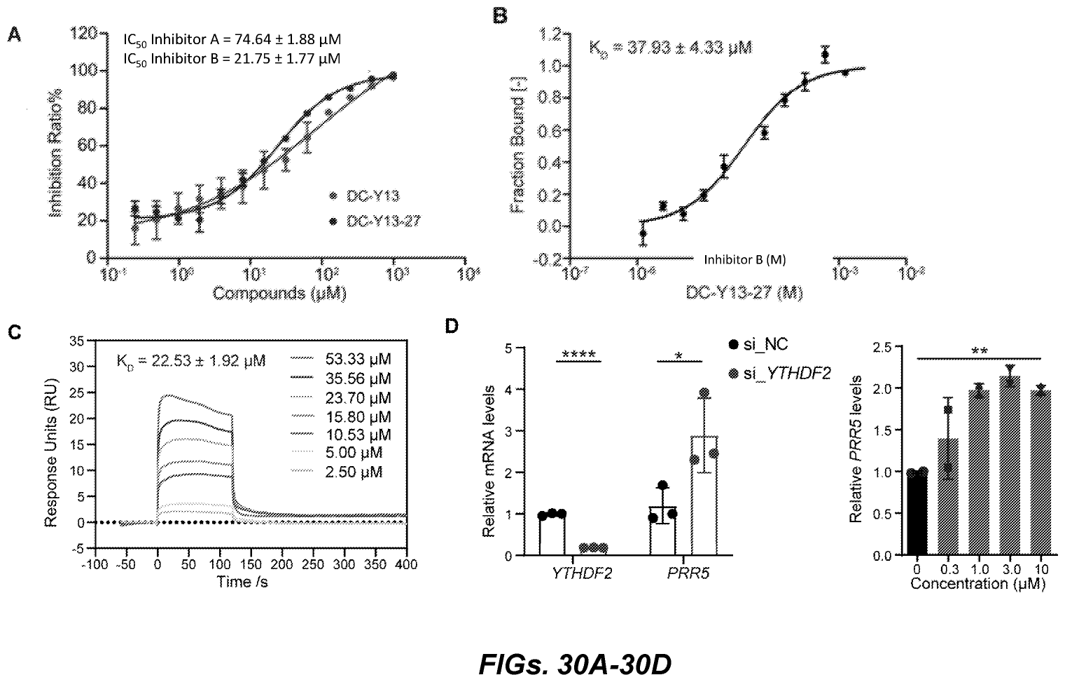

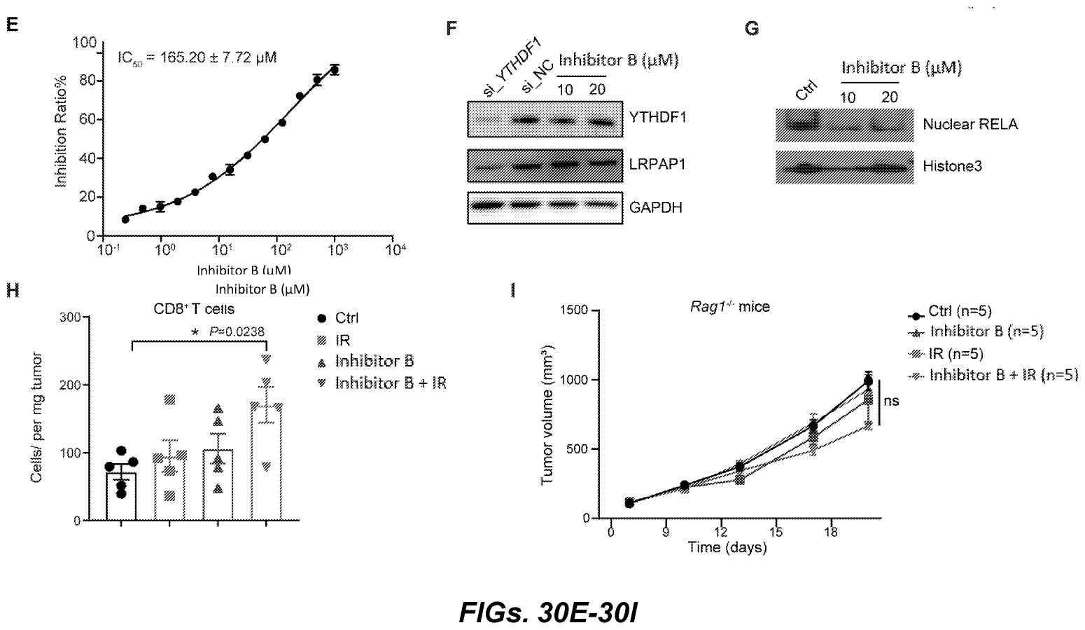

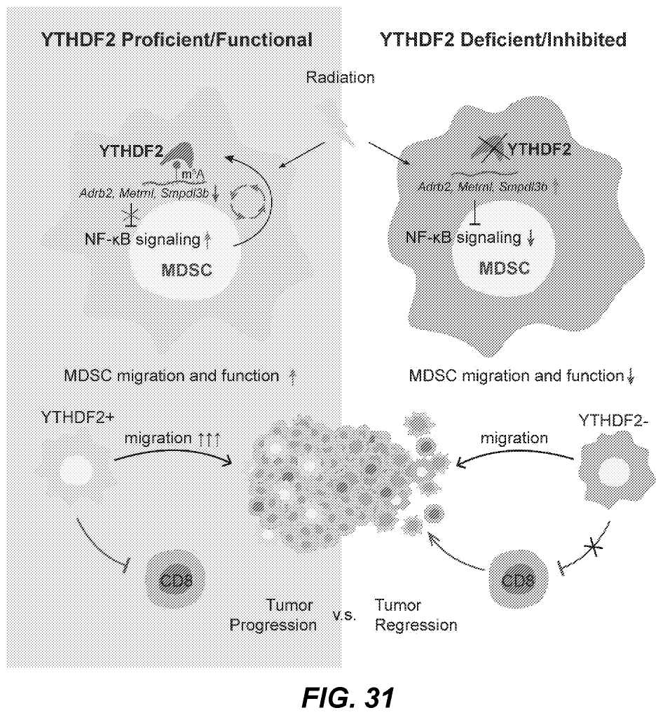

To demonstrate that enhanced efficacy of RT by YTHDF2 deficiency can be translated into a clinically relevant strategy, the inventors screened an in-house compound library with fluorescence polarization based high-throughput screening assays and found a small molecule, Inhibitor A, as an inhibitor of YTHDF2. Inhibitor A inhibits YTHDF2 binding to m⁶A-containing RNA with an IC₅₀ of 74.6±1.9 μM measured via AlphaScreen assay (FIG. 30A). Inhibitor B had a measured IC₅₀ of 21.8±1.8 μM (FIG. 30A). To further assess the capacity of Inhibitor B binding to YTHDF2, the inventors conducted a microscale thermophoresis assay and confirmed that Inhibitor B binds to YTHDF2 with a binding constant (KD) of 37.9±4.3 μM (FIG. 30B). The inventors further confirmed the specific binding using surface plasmon resonance (SPR) assay (FIG. 30C). Using the mRNA level of a direct binding target of YTHDF2 (PRR5) 72 as readout, the inventors further validated that Inhibitor B increased transcript level of YTHDF2 target at a similar level as that in YTHDF2 knockdown (FIG. 30D). The inventors next explored the inhibitory activity of Inhibitor B against YTHDF1, another member of the YT521-B homology (YTH) domain-containing proteins family to evaluate its selectivity. Inhibitor B obstructs the interaction between YTHDF1 and m⁶A with an IC₅₀ of 165.2±7.7 μM, as shown in FIG. 30E. Biologically, the inhibitor did not inhibit the expression of LRPAP1, a reported YTHDF1 target⁷³ (FIG. 30F), suggesting that Inhibitor B exhibits a preference for inhibiting YTHDF2 binding to m⁶A-modified RNA.

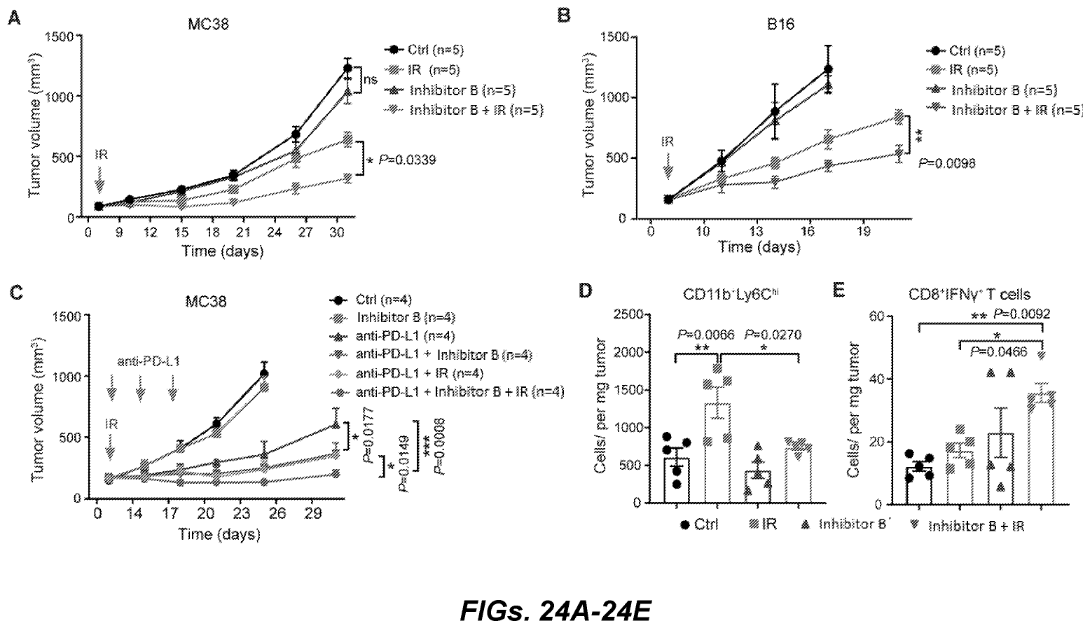

To investigate whether inhibition of YTHDF2 improves the response to IR at a similar level as Ythdf2 genetic deletion, the inventors first confirmed that the inhibitor was able to inhibit NF-κB activation (FIG. 30G). Tumor-bearing mice were treated daily with the proof-of-principle compound Inhibitor B starting on the day of IR treatment. Consistent with results obtained in Ythdf2-cKO, Inhibitor B treatment alone did not inhibit tumor growth in either the MC38 or B16 murine models. However, when combined with IR treatment, Inhibitor B treatment significantly enhanced tumor growth inhibition of IR compared with IR alone (FIG. 24A-24B). The inventors also investigated whether Inhibitor B can further increase efficacy of IR and anti-PD-L1 treatment using the MC38 model. Compared with any single treatment group, combination treatment with Inhibitor B and anti-PD-L1 resulted in significantly slower growth of MC38 tumors, and the triple therapy of Inhibitor B, IR, and anti-PD-L1 gave rise to the most robust antitumor effects (FIG. 24C).

To interrogate the underlying immunological mechanisms, the inventors profiled the tumor-infiltrating immune cells in MC38 tumors following Inhibitor B treatment by flow cytometry. The inventors observed that IR failed to increase infiltration of CD11b⁺Ly6C^{hi} cell in tumors with Inhibitor B treatment, whereas a 2-fold increase was observed following IR alone (FIG. 24D). This is consistent with the aforementioned findings in irradiated Ythdf2-cKO mice. Given the critical roles of MDSCs in determining T-cell infiltration and function, the inventors examined the CD8⁺ T-cell populations in tumors five days after IR and Inhibitor B treatment. The total numbers of both CD8⁺ T cells and IFNγ⁺CD8⁺ T cells were increased in tumors receiving the combination treatment (FIGS. 23E and 30H). This was also evidenced by the abolished antitumor efficacy of Inhibitor B plus IR in Rag1 knockout mice (FIG. 30I). The results demonstrate that the antitumor effect of the combination treatment with YTHDF2 inhibition and IR relies on adaptive immunity and is similar to that observed in Ythdf2 deleted mice.

### Example 19: Implications of Examples Described Herein

Here the inventors report that the genetic and proof-of-principle pharmacologic blockade of the m⁶A reader YTHDF2 improves local radiotherapy and combined radio-immunotherapy effects by reshaping the MDSC compartment, and inhibiting MDSC migration and suppressive functions. YTHDF2 was rapidly induced via IR-activated NF-κB/RELA, suggesting that YTHDF2 may play a critical role in the response to radiation-induced stress. Our study delineates a previously unknown link between IR stress and RNA m⁶A modification. YTHDF2 triggers degradation of transcripts encoding the negative regulators of IκBα, leading to enhanced NF-κB signaling, resulting in a positive feedback loop to sustain YTHDF2 expression. The IR-YTHDF2-NF-κB circuit in MDSCs represents a previously unrecognized mechanism of extrinsic radioresistance.

Through the use of single cell RNA-sequencing, the inventors are able to describe the murine colon tumor (MC38)-infiltrating immune cell atlas in the context of IR and also provide a reference map of differentiating and mature myeloid transcriptional states in the context of ablative IR (20 Gy). Based on the trajectory and functional gene enrichment analysis, myeloid cells differentiate/mature into distinct subpopulations as a response to radiation stress and these likely exert different functions compared with myeloid cells in a steady state. Following IR, Ythdf2 knockout affects the differentiation in both tumors and blood and thereby confers a unique mMDSC landscape. Considered together, the data suggest that the IR-YTHDF2 axis plays a critical role in regulating mMDSC differentiation. The inventors acknowledge that without in-depth lineage studies, the inventors cannot confirm the changes in development/differentiation trajectory of mMDSC in blood and tumor in relation to YTHDF2 status and radiation. Elucidation of the exact function of myeloid subpopulations and the molecular bases of heterogeneity varied in different cancer types responding to different treatments would require future investigations.

Our finding that NF-κB signaling in MDSCs is controlled in a positive feedback loop by YTHDF2, provides a fresh link between RNA m⁶A modification (YTHDF2 reader protein) and NF-κB signaling in specific immune cell populations. The RNA-seq and m⁶A target analyses provide the clues regarding the roles of YTHDF2 and NF-κB signaling in the regulation of MDSC migration and suppressive functions. The three YTHDF2 direct targets identified in this study serve as negative regulators of NF-κB signaling and may play additional roles in related biological processes.^{65,74,75} The inventors cannot rule out the possibility that these three proteins may target additional signaling involved in the regulation of MDSC suppressive functions. The inventors showed here that NF-κB plays a central role in the YTHDF2-regulated MDSC function, however, other signaling pathways, such as TNF signaling, may also contribute to the observed phenotypes. The inventors demonstrated that IL-10 was upregulated by IR in an YTHDF2-NF-κB dependent manner in MDSCs. The induced IL-10 might in turn affect NF-κB signaling since it has been shown to inhibit NF-κB activity in monocytes.⁷⁶ The inventors hypothesize that IL-10 may act in a negative feedback loop to regulate NF-κB in an YTHDF2 dependent manner, as a part of intricate network of biological responses to inflammation. The function and roles of YTHDF2 need to be further explored in other types of immune cells and/or in the context of distinct conditions.

Considering that YTHDF2 is expressed in most immune cells at different levels, the inventors speculate that YTHDF2 may affect functions of these different immune cells and YTHDF2 depletion in different immune cell types may impact host tumor immune response differently. The results provide proof-of-principle preclinical evidence that YTHDF2 inhibition with a selective small molecule inhibitor in a whole-animal setting notably improves the antitumor efficacy of radiotherapy, anti-PD-L1 immunotherapy or the combination. Therefore, the pharmacological inhibition of YTHDF2 in vivo can complement and synergize with many existing cancer therapies to overcome immunosuppression and enhance treatment efficacy and patient response rates.

Genetic YTHDF2 depletion not only enhances the local anti-tumor effects of radiation but also suppresses distant metastasis that may occur through radiation induced MDSC mobilization. The clinical importance of these observations is significant; ablation of YTHDF2 activity could be an ideal strategy of enhancing the effects of local radiotherapy as well as suppression of distant metastasis. Radiation-induced YTHDF2 expression may also explain the failure to induce a consistent abscopal effect—a rare phenomenon involving antitumor effect on distant metastasis following irradiation of a single lesion—as well as the failure to improve survival in many radiotherapy/checkpoint inhibitor trials. YTHDF2 blockade presents a potential paradigm shift in radiosensitization, in that not only are the antitumor effects of radiotherapy enhanced in treated tumors, but also that local radiation can be modified to suppress induction of distant metastasis.

### Example 20: Further Experimental Methods, Models, and Subject Details

### Cells

MC38 and B16 were purchased from ATCC and were maintained according to the method of characterization used by ATCC. LLC cells were obtained from American Type Culture Collection (CRL-1642). B16-OVA were selected as single clones with 5 μg/ml puromycin (InvivoGen) after stable infection with lentivirus-expressing OVA protein. Cells were grown in Dulbecco's modified Eagle's medium (DMEM, Gibco) containing 10% heat-inactivated fetal bovine serum (FBS, Gemini),

Penicillin (100 U/mL)/Streptomycin (100 ug/mL, Gibco), and were maintained in a humidified incubator with 5% CO₂ at 37° C.

### Mice

All mice were housed and used according to the animal experimental guidelines set by the Institute of Animal Care and Use Committee of The University of Chicago. All animals were maintained in pathogen-free conditions and cared for in accordance with the International Association for Assessment and Accreditation of Laboratory Animal Care policies and certification. Ythdf2^{flox/flox} mice were generated using CRISPR-Cas9 technology as described.⁷⁹ Lyz^{Cre} mice, Cd45.1 mice, Ccr2^{-/-} mice, Nfkb1^{-/-} mice and Rag1^{-/-} mice were purchased from The Jackson Laboratory. Male and female mice aged eight to ten weeks were used in the experiments.

### Patient Sample

Patient samples (PBMCs) were obtained from patients treated at the University of Chicago enrolled in the trials NCT02608385⁵⁶ and NCT03223155⁵⁷. Pembro-SBRT study (NCT02608385) and COSINR study (NCT03223155): The studies and amendments were approved by the University of Chicago Biological Sciences Division institutional review board (IRB15-1130 and IRB17-0547, respectively). The studies complied with all ethical regulations and all patients provided written informed consent.

### Tumor Growth and Treatment

MC38, LLC, B16 or B16-OVA tumor cells were subcutaneously (s.c.) injected in the right flank of mice. For tumor fragment model, MC38 tumors were excised and cut off into fragments, and implanted subcutaneously into recipient mice. Mice were pooled and randomly divided into different groups when the tumor reached a volume of approximately 100 mm³ (L×W×H×0.5). The mice were treated with 20 Gy of tumor-localized radiation (one dose) or sham treatment. For anti-PD-L1 treatment experiments, 200 μg of the anti-PD-L1 antibody were injected intraperitoneally twice each week for a total of four times. For YTHDF2 inhibitor treatment, 9 μg of the inhibitor were intravenously injected every day. Tumors were measured twice one week for 3-4 weeks. Animals were euthanized when the tumor volume reached 2,000 mm³ or the diameter of tumor reached 1.5 cm (according to the IACUC protocol). For CD8⁺ T cell depletion experiments, 200 μg of anti-CD8α antibody were delivered by intraperitoneal injection, start from one day before other treatments (twice a week).

### Flow Cytometry

For flow cytometric analysis, tumors, lymph nodes, spleens or blood were collected from mice. The collected tumors tissues were cut into small pieces and were digested with 1 mg/ml collagenase type I or IV (Fisher) and 200 μg/ml DNaseI (Sigma-Aldrich) at 37° C. for 60 min to generate the single-cell suspensions. Cells from spleens or lymph nodes were isolated by grinding the tissues through 70 μm filters. Samples were then filtered through a 70 μm cell strainer and washed twice with staining buffer (PBS supplemented with 2% FBS and 0.5 mM EDTA). The cells were re-suspended in staining buffer and were blocked with anti-FcR (2.4G2, BioXcell). Subsequently, the cells were stained with 200-fold diluted fluorescence-labeled antibodies for 30 min at 4° C. in the dark and then detected by flow cytometry with a BD Fortessa (BD). For intracellular staining, cells were first permeabilized using a Fixation and Permeabilization Kit (BD) and then stained with appropriate antibodies. Analysis of flow cytometry data was performed using FlowJo V10.

For intracellular staining of YTHDF2, the tumor infiltrating cells were first fixed with Fixation Buffer (BD) for 60 min on ice, and then washed twice with diluted Permeabilization Buffer (BD). Then anti-mouse YTHDF2 antibody (Abcam, ab220163) were added and incubated at 4° C. overnight, followed by adding the Alexa Flour 647 goat anti-rabbit IgG (Life technologies) and staining for 60 min.

### ELISPOT Assay

For CD8⁺ T cell functional assay, CD8⁺ T cells were isolated from MC38-OVA tumors, seven days after IR. 2-4×10⁵ CD8⁺ T cells were re-stimulated with/without 1 μg/ml SIINFEKEL. After 48-72 hr incubation, the cells were removed. Alternatively, CD11c⁺ DCs were sorted from naïve mice and co-cultured with irradiated tumor cells for 6 hr; then DCs were purified and co-cultured with isolated CD8⁺ T cells for another 48-72 hr. The cytokine spots of IFN-γ were detected with an IFN-γ ELISPOT assay kit according to product protocol. IFN-γ spots were developed according to the manufacturer's instructions (BD) and calculate by ELISPOT Reader.

### ELISA

For IL-10 ELISA assay, tumor tissues were collected three days after IR from tumor-bearing WT or Ythdf2-cKO mice and were homogenized in PBS with protease inhibitor (1:100). The concentration of IL-10 was measured with an IL-10 Mouse ELISA Kit (Abcam) in accordance with the manufacturer's instruction.

### Cytokine Detection

For IFN-γ and TNF-α detection, MC38 tumors were collected from WT or Ythdf2-cKO mice three days after IR. Tumor tissues were homogenized in PBS with protease inhibitor (1:100), and then centrifuged at 12,000 rpm for 10 min to collect the supernatant. The supernatant was used to detect the cytokines with LEGENDplex™ Mouse Inflammation Panel (13-plex) with V-bottom Plate kit (BioLegend). The samples were detected by flow cytometry with a BD Fortessa (BD). The obtained flow data was analyzed with LEGENDplex software (v8.0, BioLegend).

### BM-MDSCs Induction and Isolation

Bone marrow was obtained from wild type. WT or Ythdf2-cKO mice and was used to prepare single cell suspension. The cell suspension was called fresh bone marrow cells. The cells were cultured in RPIM-1640 medium containing 10% FBS and 20 ng/ml Recombinant Mouse GM-CSF carrier-free (BioLegend). Fresh medium supplemented with GM-CSF was added on day 3. On day 4, the bone marrow-derived MDSCs (BM-MDSCs) were obtained from fresh bone marrow cells followed with MDSCs isolation using EasySep Selection kits (STEMCELL Technologies).

### MDSC Suppression Assay

Murine MDSCs purified from tumors or bone marrow derived MDSCs were performed for the suppression assay. CD8⁺ T cells isolated from the spleen of naïve mice by using EasySep™ Mouse CD8⁺ T Cell Isolation Kit (STEMCELL) according to manufacturer's instructions and then stained with CellTrace CFSE (Invitrogen). The CD8⁺ T cells were cultured with anti-CD3/anti-CD28 beads and were co-cultured with MDSCs at a ratio of 4:1. The CD8⁺ T cells proliferation was analyzed by flow cytometry.

### Knockdown in BM-MDSCs

SiRNA targeting mouse Adrb2, Metrnl, or Smpdl3b respectively was transfected into bone marrow derived MDSCs by TransIT-TKO® Transfection Reagent (Mirus) according to manufacturer's protocol. The sequences of siRNA are mouse Adrb2: 5′-UAA CAA UCG AUA GCU UUC Utg-3′; mouse Metrn1: 5′-UUG AAA GUC ACU AAA GCG Ugg-3′; mouse Smpdl3b 5′-UUU GGA UAG GGU GUA GUU Ggg-3′. One-two days after the transfection, the cells were collected. The knockdown efficiency was detected by qPCR.

### Transwell Migration Assay

The inventors used 6-well or 24-well transwell plates with 8 μm inserts in polyethylene terephthalate track-etched membranes (Corning). The purified MDSCs from tumors or bone marrow derived cells (5.0×10⁶ cells/insert for 6-well; 1.5×10⁶ cells/insert for 24-well) in serum-free medium were added into the upper compartment of the chamber. The inserts were placed in plates with complete DMEM medium. After incubating overnight, insert membranes were washed with PBS, fixed with 70% methanol for 10 min, and stained with 0.05% crystal violet to detect the migrated cells. An inverted microscope was used for counting.

### RNA Stability Assay

MDSCs were sorted from spleen in WT or Ythdf2-cKO mice and were seeded in 24-well plates at 50% confluency. 5 μg/mL of Actinomycin D (Sigma-Aldrich) was added. After 0, 0.5, 1, 3, and 6 hours of incubation, cells were collected. The total RNA was purified by RNeasy kit with an additional DNase-I digestion step on the column. RNA quantities were determined using RT-qPCR analysis.

### Forced Expression of YTHDF2 in MDSCs

The cloned Ythdf2 cDNA with K416A, R527A, W432A, and W486A mutation, which has been proved to significantly decrease the m⁶A binding affinity⁸⁰, were synthesized and cloned into the lentiviral expression vector pLVX-ZsGreen-N1 to generate pLVX-ZsGreen-N1-YTHDF2-Mut (GenScript). The constructed vectors were packaged by co-transfection of 293X cells with two lentiviral helper plasmids pVSVG and pVPR. Virus-containing conditioned medium was harvested 48 h after transfection, filtered, and used to infect BM-MDSCs in the presence of 8 μg/mL polybrene. Infected cells were selected with 2 μg/mL puromycin.

### RIP-Seq

The tumor infiltrated CD11b⁺ myeloid cells were sorted using the EasySep Selection kits (STEMCELL Technologies) from five pooled wild-type (Ythdf2^{f/f}) mice three days after IR per technical replicate (total three technical replicates). The purified cells were washed with cold PBS and the cell pellet was re-suspended with three packed cell volume of lysis buffer (150 mM KCl, 10 mM HEPES pH 7.5, 2 mM EDTA, 0.5% NP-40, 0.5 mM dithiothreitol (DTT), 1:100 protease inhibitor cocktail, 400 U/ml RNase inhibitor), pipetted up and down several times and incubated on ice for 30 min, and treated with ultrasonic for 1 min. The lysate was centrifuged for 30 min at 1, 4000 rpm (4° C.) to clear the lysate. One-tenth volume of cell lysate was saved as input and mixed with Trizol to extract the total RNA. The rest of the cell lysate was incubated with 20 μg anti-YTHDF2 rabbit polyclonal antibody (Aviva systems biology) at 4° C. overnight with gentle rotation. 200 μL protein G beads were thrice washed with binding buffer, and incubated with cell lysate-antibody mixture at 4° C. for at least 4 h. Then, the protein G beads were collected with magnetic stand, thrice washed with binding buffer, and mixed with Trizol for RNA extraction and saved as IP. Subsequently, the RNA library for sequencing was constructed using SMARTer® Stranded Total RNA-Seq Kit v2—Pico Input Mammalian (Takara Bio).

### RIP-qPCR Analysis

RIP for YTHDF2 was performed using 20 μg anti-YTHDF2 rabbit polyclonal antibody (Aviva systems biology), as described above. After IP, RNA was isolated from Input and IP fractions using phenol/chloroform extraction. cDNA was prepared with the Applied Biosystems™ High-Capacity cDNA Reverse Transcription Kit (Thermo). SYBR-green-based qPCR was performed using QuantiStudio3 (ABI).

m6A-Seq

Total RNA was isolated from tumor infiltrated CD11b⁺ myeloid cells and followed by two rounds of ploy (A) selection to get mRNA. CD11b⁺ myeloid cells were sorted from five pooled Ythdf2^{f/f} mice three days after IR per technical replicate (total three technical replicates). The 100 ng mRNA was used for m⁶A immunoprecipitation (m⁶A-IP) with the EpiMark N⁶-methyladenosine enrichment kit (NEB E1610S) according to the manufacturer's protocol. The library was constructed using SMARTer® Stranded Total RNA-Seq Kit v2—Pico Input Mammalian (TaKaRa Bio) and the sequencing was performed at the University of Chicago Genomics Facility on an Illumina NovaSEQ machine in pair-read mode with 100 bp per read.

Single Cell RNA-Seq (scRNA) Analysis

Regarding the CD45⁺ immune cells scRNA-seq, single-cell suspensions were obtained from four pooled MC38 tumors in WT mice with or without IR (20 Gy) four days after IR. Samples were stained using Zombie Red™ dye (for live cells) for 30 min and then stained for 20 min using an antibody against mouse CD45. Zombine Red⁻CD45⁺ single cells were sorted for library construction of scRNA-seq. Regarding the mMDSCs scRNA-seq, single-cell suspensions were obtained from pooled MC38 tumors in five WT or Ythdf2-cKO mice with or without IR (20 Gy) respectively three days after IR. Samples were stained using Zombie Red™ dye for 30 min and then stained for 20 min using an antibody against oligo-conjugated antibodies mouse CD45, CD11b, Ly6C respectively (TotalSeq™-B). Zombie Red⁻CD45⁺CD11b⁺Ly6C^{hi} single cells were sorted for library construction of scRNA-seq. The library construction was performed at the University of Chicago Genomics Facility, using Chromium Next GEM Single Cell 3′ GEM, Library & Gel Bead Kit v3.1 (Cat: 1000128) purchased from 10× Genomics according to the protocols provided by manufactures. The aimed target cell recovery for each library was 8,000 and the libraries were sequenced on an Illumina HiSeq X Ten platform at the University of Chicago Genomics Facility.

Raw scRNA-seq data were processed using 10× Genomics Cell Ranger (v6.0.1), including demultiplexing Illumina base call files (BCL) into FASTQ files (with “cellranger mkfastq” function), aligning sequencing reads in FASTQ files to the mouse reference genome (mm10, GENCODE vM23/Ensembl 98 released on Jul. 7, 2020, from 10× Genomics) and counting the unique molecular identifier (UMI) (with “cellranger count” function). As a results, the inventors generated the digital gene expression matrix with the number of UMIs for each gene in each cell.

Low-quality cells were discarded if (1) the number of expressed genes was smaller than 200; (2) the proportion of mitochondrial gene expression were larger than 25%. The inventors further identified and removed potential doublets by using DoubletFinder (v2.0.3) assuming 6% doublet formation rate.⁸¹ For scRNA-seq of mMDSC, as the inventors applied Cell Hashing method using a series of oligo-tagged antibodies against ubiquitously expressed surface proteins with different barcodes to uniquely label cells from distinct samples, the inventors demultiplex cells into different samples based on HTO enrichment by using HTODemux method in Seurat package (v4.0.6).⁸²

The processed whole gene expression matrix was then fed to Seurat (v4.0.6) for downstream analyses.⁸² Briefly, only genes expressed in more than 3 cells were kept, and the UMI count matrix was normalized by using ‘NormalizeData’ function. Later, 2,000 highly variable genes were identified by using the ‘FindVariableFeatures’ function with the ‘vst’ method, and ‘ScaleData’ function was applied to scale and center the gene expression matrix. Clustering analyses were performed using the first 40 principal components for constructing the shared nearest neighbor (SNN) graph by using ‘FindNeighbors’ function, and then Louvain clustering algorithm was used to group the cells into different clusters. Next, the inventors applied scClassify (v1.2.0)⁸³ for cell type classification based on cell types hierarchies constructed from reference datasets (E-MTAB-8832, CD45⁺ immune cells sorted from MC38 tumor-bearing C57BL/6 mice).⁸⁴

### Bulk RNA-Seg Analysis

Raw reads were trimmed with Trimmomatic-0.39⁸⁵, then aligned to mouse genome and transcriptome (mm10, version M19, 2018 Aug. 30) using HISAT (version 2.1.0)⁸⁶ with ‘-rna-strandness RF’ parameters. Annotation files (version M19, 2018 Aug. 30, in gtf format for mouse) were downloaded from GENCODE database (https://www.gencodegenes.org/). For mRNA m⁶A-seq, mapped reads were separated by strands with samtools (version 1.9)⁸⁷ and m⁶A peaks on each strand were called using MACS (version 2)⁸⁸ with parameter ‘-nomodel, --keep-dup 5, -g 2.052e9, --tsize 114-extsize 150’ separately. Significant peaks with q<0.01 identified by MACS2 were considered. Peaks identified in at least three biological replicates were merged using bedtools (v.2.26.0)⁸⁷ and were used in the following analysis. Reads, from input of m⁶A-seq or YTHFDF2 RIP-seq, on each GENCODE annotated gene were counted using HTSeq⁸⁹ and then differentially expressed genes were called using DESeq2 package in R⁹⁰ requiring at least 10 read counts in at least three samples with adjusted p-value<0.05. YTHDF2 target genes were identified as differentially up-regulated genes comparing YTHDF2 IP sample with the corresponding Input samples. Functional enrichment analysis was performed with DAVID⁹¹.

### TCGA Data Analysis

TCGA data was acquired and analyzed in part using the Xena Platform.⁹² For survival analysis an MDSC signature score based on expression of ARG1, CD14, CD44, CD40, S100A8, SELPLG, STAT6, TFRC, TGFB2, STAT3, CD274, ITGA3, SLA, and KDR was applied to the TCGA data. For the Low Grade Glioma (LGG) and Glioblastoma (GBM) cohorts, gene expression was divided into the highest and lowest thirds. Samples in the lowest and highest groups received a score of −1 and 1, respectively. Scores were then added for each sample and samples with a final score within the highest or lowest third was given a ‘High’ or ‘Low’ MDSC signature score, respectively. For all cohorts samples were filtered by ‘Primary Tumor’ and for patients receiving radiation therapy. Survival data from these groups were then compared and significance calculated using the ggsurvplot function in R.

### Chromatin Immunoprecipitation (ChIP) Assay

ChIP assays were conducted with a Magna ChIP™ A/G Chromatin Immunoprecipitation Kit (Sigma/Millipore). Briefly, 5-10×10⁶ BM-MDSCs were fixed with a final concentration of 1% formaldehyde, cross-linked, and sonicated. The anti-RELA antibody (10 μg/mL, CST), or IgG control antibody was added to sonicated lysates and incubated overnight at 4° C., then incubated with Protein A/G beads mixture (1:1 at ratio) for another >7 h at 4° C. Chromatin DNA was eluted, reverse cross-linked, and recovered using a QIAquick Extraction Kit (Qiagen). Input DNA and immunoprecipitated DNA were analyzed by quantitative PCR using the Ythdf2 promoter DNA-specific primers.

### Protein Expression and Purification

YTHDF2 (aa 380-559) and YTHDF1 (aa 361-559) were first cloned into modified pET28a-TEV vector. The plasmid was transformed into E. coli BL21 (DE3) cells and the proteins were induced with 1 mM Isopropyl-D-thiogalactopyranoside (IPTG) for 16 hours at 20° C. The cells were collected and resuspended in the lysis buffer containing 20 mM Tris (pH 7.4), 150 mM NaCl, 0.05% (v/v)-mercaptoethanol and 5% (v/v) glycerol. YTHDF2 (aa 380-579) and YTHDF1 (aa 361-559) were then purified through Ni-NTA chromatography (HisTrap FF, GE Healthcare), followed by the purifications including a cation exchange column and a Superdex 75 10/300 column. The purified proteins were stored at −80° C. in the buffer containing 20 mM Hepes (pH 7.4) and 200 mM NaCl.

### Fluorescence Polarization (FP)

The high throughput screening (HTS) of the laboratory's in-house compound library was performed at the final concentration of 80 μM. Diluted compounds were first incubated with 1.25 μM YTHDF2 (aa 380-579) for half an hour at 25° C. in the binding buffer containing 20 mM Hepes (pH 7.4), 50 mM NaCl, 5% (v/v) glycerol and 0.01% (v/v) tween 20. And 30 nM fluorescently-labeled m6A-containing mRNA (5′-FAM-UUCUUCUGUGG (m6A) CUGUG-3′) was then added and incubated for another one hour at 4° C. before testing via Envision Readers (PerkinElmer). The same amount of DMSO was used as the negative control, unlabeled m⁶A-containing mRNA with the same sequence was used as the positive control and the 5′-FAM-labeled m⁶A-containing mRNA was utilized to ascertain the gain factor.

### AlphaScreen

The compound Inhibitor A and Inhibitor B were first diluted from 1 mM to concentrations as indicated using double dilution method, respectively. Then His-tagged YTHDF2 (aa 380-579) or His-tagged YTHDF1 (aa 361-559) was added to the diluted compounds at the final concentration of 80 nM. The same amount of DMSO and the unlabeled m⁶A-containing mRNA were separately served as the negative control and the positive control, respectively. The samples were incubated in the binding buffer containing 20 mM Hepes (pH 7.4), 150 mM NaCl, 0.01% (v/v) TritonX-100 and 1 mg/ml BSA for half an hour at 25° C. before biotinylated m⁶A-containing mRNA (5′-biotin-UUCUUCUGUGG (m⁶A) CUGUG-3′) was added at the final concentration of 10 nM. Next, the mixture of anti-His acceptor beads and streptavidin donor beads were added away from light. And the samples were then incubated for another one hour at 4° C. and then measured on Envision Readers (PerkinElmer).

### Microscale Thermophoresis (MST)

The compound Inhibitor B was first diluted from 2.5 mM using a double dilution method with the MST assay buffer containing 20 mM Hepes (pH 7.4), 200 mM NaCl and 0.1% (v/v) Pluronic® F-127. Then, the compound samples were incubated with YTHDF2 (aa 380-579) at the final concentration of 2 μM for 20 minutes at 25° C. followed by 10 minutes of 13,000 rpm centrifugation at 4° C. before the detection. Next, prepared samples were loaded into the Monolith NT. Automated LabelFree Premium Capillary Chips (NanoTemper Technologies) and the experiments were performed using the label-free method on the Monolith NT. Automated instrument (NanoTemper Technologies). The binding constant (KD) of Inhibitor B and YTHDF2 (aa 380-579) was acquired by analyzing data using the MO. Affinity Analysis Software v2.3 (NanoTemper Technologies).

### Method-Surface Plasmon Resonance Assay

Surface plasmon resonance assay was performed to test the binding affinity of YTHDF2 protein and Inhibitor B on a Biacore T200 instrument (GE Healthcare) with HBS buffer (20 mM HEPES pH7.4, 200 mM NaCl, 0.08% (v/v) DMSO) at 25° C. The protein was immobilized on a CM5 chip (GE Healthcare) using a standard amine-coupling procedure in 10 mM sodium acetate (pH 4.0). The chip was then equilibrated in HBS buffer. The compound was serially dissolved with HBS buffer. For each cycle, compound solution was injected for 120 s, followed by a 300 s delay for dissociation. The KD values were determined by Biacore T200 evaluation software (GE Healthcare).

### Quantification And Statistical Analysis

To estimate the statistical significance of differences between two groups, the inventors used a paired or un-paired Student's t-tests to calculate two-tailed P values. One-way analysis of variance (ANOVA) or two-way ANOVA with multiple comparison test was performed when more than two groups were compared. Survival analysis was performed using Kaplan-Meier curves and evaluated with log-rank Mantel-Cox tests. Error bars indicate the standard error of the mean (SEM) unless otherwise noted. P values are labeled in the figures. P values were denoted as follows: *P<0.05. **P<0.01. ***P<0.001, ****P<0.0001. Statistical analyses were performed by using GraphPad Prism (version 8.0).

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barker H E, Paget J T, Khan A A, Harrington K J. The tumour microenvironment after radiotherapy: mechanisms of resistance and recurrence. Nat Rev Cancer. 2015 July; 15(7): 409-25. doi: 10.1038/nrc3958. Erratum in: Nat Rev Cancer. 2015 August; 15(8): 509.
Brach M A, Hass R, Sherman M L, Gunji H, Weichselbaum R. Kufe D. Ionizing radiation induces expression and binding activity of the nuclear factor kappa B. J Clin Invest. 1991 August; 88(2): 691-5.
Califano J A. Khan Z, Noonan K A, Rudraraju L, Zhang Z. Wang H, Goodman S, Gourin C G, Ha P K, Fakhry C, Saunders J, Levine M, Tang M, Neuner G, Richmon J D, Blanco R. Agrawal N, Koch W M, Marur S, Weed D T, Serafini P. Borrello I. Tadalafil augments tumor specific immunity in patients with head and neck squamous cell carcinoma. Clin Cancer Res. 2015 Jan. 1; 21(1): 30-8.
Chen M, Qiao G, Hylander B L, Mohammadpour H, Wang X Y, Subjeck J R. Singh A K, Repasky E A. Adrenergic stress constrains the development of anti-tumor immunity and abscopal responses following local radiation. Nat Commun. 2020 Apr. 14; 11(1): 1821.
Cheng Q J, Ohta S. Sheu K M, Spreafico R, Adelaja A. Taylor B. Hoffmann A. NF-κB dynamics determine the stimulus specificity of epigenomic reprogramming in macrophages. Science. 2021 Jun. 18; 372(6548): 1349-1353.
Condamine T, Mastio J, Gabrilovich D I. Transcriptional regulation of myeloid-derived suppressor cells. J Leukoc Biol. 2015 December; 98(6): 913-22.
Dixit D, Prager B C, Gimple R C, Poh H X, Wang Y, Wu Q. Qiu Z, Kidwell R L, Kim L J Y, Xie Q. Vitting-Seerup K, Bhargava S, Dong Z, Jiang L, Zhu Z, Hamerlik P, Jaffrey S R, Zhao J C, Wang X, Rich J N. The RNA m6A Reader YTHDF2 Maintains Oncogene Expression and Is a Targetable Dependency in Glioblastoma Stem Cells. Cancer Discov. 2021 February; 11(2): 480-499.
Engblom C, Pfirschke C, Pittet M J. The role of myeloid cells in cancer therapies. Nat Rev Cancer. 2016 July; 16(7): 447-62.
Erstad D J, Cusack J C Jr. Targeting the NF-κB pathway in cancer therapy. Surg Oncol Clin N Am. 2013 October; 22(4): 705-46.
Feng S, Cheng X, Zhang L, Lu X, Chaudhary S, Teng R, Frederickson C, Champion M M, Zhao R, Cheng L, Gong Y, Deng H, Lu X. Myeloid-derived suppressor cells inhibit T cell activation through nitrating LCK in mouse cancers. Proc Natl Acad Sci USA. 2018 Oct. 2; 115(40): 10094-10099.
Frye M, Harada B T, Behm M, He C. RNA modifications modulate gene expression during development. Science. 2018 Sep. 28; 361(6409): 1346-1349.
Gabrilovich D I, Nagaraj S. Myeloid-derived suppressor cells as regulators of the immune system. Nat Rev Immunol. 2009 March; 9(3): 162-74.
Gentles A J, Newman A M, Liu C L, Bratman S V, Feng W, Kim D, Nair V S, Xu Y, Khuong A, Hoang C D, Dichn M, West R B, Plevritis S K, Alizadeh A A. The prognostic landscape of genes and infiltrating immune cells across human cancers. Nat Med. 2015 August; 21(8): 938-945.
Gray J E, Villegas A, Daniel D, Vicente D, Murakami S, Hui R, Kurata T, Chiappori A. Lee K H, Cho B C, Planchard D, Paz-Ares L, Faivre-Finn C, Vansteenkiste J F, Spigel D R. Wadsworth C, Taboada M, Dennis P A, Özgüroğlu M, Antonia S J. Three-Year Overall Survival with Durvalumab after Chemoradiotherapy in Stage III NSCLC-Update from PACIFIC. J Thorac Oncol. 2020 February; 15(2): 288-293.
Halama N, Zoernig I, Berthel A, Kahlert C, Klupp F, Suarez-Carmona M, Suetterlin T, Brand K. Krauss J, Lasitschka F, Lerchl T, Luckner-Minden C, Ulrich A, Koch M, Weitz J, Schneider M, Buechler M W, Zitvogel L, Herrmann T, Benner A, Kunz C, Luecke S, Springfeld C. Grabe N, Falk C S, Jaeger D. Tumoral Immune Cell Exploitation in Colorectal Cancer Metastases Can Be Targeted Effectively by Anti-CCR5 Therapy in Cancer Patients. Cancer Cell. 2016 Apr. 11; 29(4): 587-601.
Han D, Liu J, Chen C, Dong L, Liu Y. Chang R, Huang X, Liu Y, Wang J. Dougherty U, Bissonnette M B, Shen B, Weichselbaum R R, Xu M M, He C. Anti-tumour immunity controlled through mRNA m6A methylation and YTHDF1 in dendritic cells. Nature. 2019 February; 566(7743): 270-274.
Han T, Guo M, Gan M, Yu B, Tian X, Wang J B. TRIM59 regulates autophagy through modulating both the transcription and the ubiquitination of BECN1. Autophagy. 2018; 14(12): 2035-2048.
Heinz L X, Baumann C L, Koberlin M S, Snijder B, Gawish R. Shui G, Sharif O, Aspalter I M, Müller A C, Kandasamy R K, Breitwieser F P, Pichlmair A, Bruckner M, Rebsamen M, Blüml S, Karonitsch T, Fauster A, Colinge J, Bennett K L, Knapp S, Wenk M R, SupertiFurga G. The Lipid-Modifying Enzyme SMPDL3B Negatively Regulates Innate Immunity. Cell Rep. 2015 Jun. 30; 11(12): 1919-28.
Hesser C R, Karijolich J, Dominissini D, He C. Glaunsinger B A. N6-methyladenosine modification and the YTHDF2 reader protein play cell type specific roles in lytic viral gene expression during Kaposi's sarcoma-associated herpesvirus infection. PLOS Pathog. 2018 Apr. 16; 14(4): e1006995.
Hou J, Zhang H, Liu J, Zhao Z, Wang J, Lu Z. Hu B. Zhou J, Zhao Z, Feng M, Zhang H, Shen B, Huang X, Sun B. Smyth M J, He C, Xia Q. YTHDF2 reduction fuels inflammation and vascular abnormalization in hepatocellular carcinoma. Mol Cancer. 2019 Nov. 18; 18(1): 163.
Hou Y. Liang H, Rao E, Zheng W. Huang X, Deng L, Zhang Y, Yu X, Xu M, Mauceri H. Arina A, Weichselbaum R R, Fu Y X. Non-canonical NF-κB Antagonizes STING Sensor-Mediated DNA Sensing in Radiotherapy. Immunity. 2018 Sep. 18; 49(3): 490-503.c4.
Iclozan C, Antonia S, Chiappori A, Chen D T, Gabrilovich D. Therapeutic regulation of myeloid-derived suppressor cells and immune response to cancer vaccine in patients with extensive stage small cell lung cancer. Cancer Immunol Immunother. 2013 May; 62(5): 909-18.
Luke J J, Lemons J M, Karrison T G, Pitroda S P, Melotek J M, Zha Y, Al-Hallaq H A, Arina A, Khodarev N N, Janisch L, Chang P, Patel J D, Fleming G F, Moroney J, Sharma M R, White J R, Ratain M J, Gajewski T F, Weichselbaum R R, Chmura S J. Safety and Clinical Activity of Pembrolizumab and Multisite Stereotactic Body Radiotherapy in Patients With Advanced Solid Tumors. J Clin Oncol 2018 Jun. 1; 36(16): 1611-1618
Jiang P. Gu S, Pan D, Fu J, Sahu A, Hu X, Li Z, Traugh N, Bu X, Li B, Liu J, Freeman G J, Brown M A, Wucherpfennig K W, Liu X S. Signatures of T cell dysfunction and exclusion predict cancer immunotherapy response. Nat Med. 2018 October; 24(10): 1550-1558.
Jung T W, Pyun D H, Kim T J, Lec H J, Park E S, Abd El-Aty A M, Hwang E J, Shin Y K. Jeong J H. Meteorin-like protein (METRNL)/IL-41 improves LPS-induced inflammatory responses via AMPK or PPARδ-mediated signaling pathways. Adv Med Sci. 2021 March; 66(1): 155-161.
Karakasheva T A, Waldron T J, Eruslanov E, Kim S B, Lee J S, O'Brien S, Hicks P D, Basu D, Singhal S, Malavasi F. Rustgi A K. CD38-Expressing Myeloid-Derived Suppressor Cells Promote Tumor Growth in a Murine Model of Esophageal Cancer. Cancer Res. 2015 Oct. 1; 75(19): 4074-85.
Kelly R J, Ajani J A, Kuzdzal J, Zander T, Van Cutsem E, Piessen G, Mendez G, Feliciano J, Motoyama S, Lièvre A, Uronis H, Elimova E, Grootscholten C. Geboes K, Zafar S, Snow S, Ko A H, Feeney K, Schenker M, Kocon P, Zhang J, Zhu L, Lei M, Singh P, Kondo K, Cleary J M, Mochler M; CheckMate 577 Investigators. Adjuvant Nivolumab in Resected Esophageal or Gastroesophageal Junction Cancer. N Engl J Med. 2021 Apr. 1; 384(13): 1191-1203.
Ko J S, Zea A H, Rini B I, Ireland J L, Elson P, Cohen P. Golshayan A, Rayman P A, Wood L, Garcia J, Dreicer R, Bukowski R. Finke J H. Sunitinib mediates reversal of myeloid-derived suppressor cell accumulation in renal cell carcinoma patients. Clin Cancer Res. 2009 Mar. 15; 15(6): 2148-57.
Kumar V, Patel S, Tcyganov E, Gabrilovich D I. The Nature of Myeloid-Derived Suppressor Cells in the Tumor Microenvironment. Trends Immunol. 2016 March; 37(3): 208-220.
Kwon E D, Drake C G, Scher H I, Fizazi K, Bossi A, van den Eertwegh A J, Krainer M, Houede N, Santos R, Mahammedi H, Ng S, Maio M, Franke F A, Sundar S, Agarwal N, Bergman A M, Ciuleanu T E, Korbenfeld E, Sengeløv L, Hansen S, Logothetis C, Beer™, McHenry M B, Gagnier P, Liu D, Gerritsen W R; CA184-43 Investigators. Ipilimumab versus placebo after radiotherapy in patients with metastatic castration-resistant prostate cancer that had progressed after docetaxel chemotherapy (CA 184-43): a multicentre, randomised, double-blind, phase 3 trial. Lancet Oncol. 2014 June; 15(7): 700-12.
Law A M K, Valdes-Mora F, Gallego-Ortega D. Myeloid-Derived Suppressor Cells as a Therapeutic Target for Cancer. Cells. 2020 Feb. 27; 9(3): 561.
Lee N Y, Ferris R L, Psyrri A, Haddad R I, Tahara M, Bourhis J, Harrington K, Chang P M, Lin J C, Razaq M A, Teixeira M M, Lovey J, Chamois J, Rueda A, Hu C, Dunn L A, Dvorkin M V, De Beukelaer S, Pavlov D, Thurm H, Cohen E. Avelumab plus standard-of-care chemoradiotherapy versus chemoradiotherapy alone in patients with locally advanced squamous cell carcinoma of the head and neck: a randomised, double-blind, placebo-controlled, multicentre, phase 3 trial. Lancet Oncol. 2021 April; 22(4): 450-462.
Lei Q, Wang D. Sun K, Wang L, Zhang Y. Resistance Mechanisms of Anti-PD1/PDL1 Therapy in Solid Tumors. Front Cell Dev Biol. 2020 Jul. 21; 8:672.
Liang H, Deng L, Hou Y, Meng X, Huang X, Rao E, Zheng W, Mauceri H, Mack M, Xu M, Fu Y X, Weichselbaum R R. Host STING-dependent MDSC mobilization drives extrinsic radiation resistance. Nat Commun. 2017 Nov. 23; 8(1): 1736.
Liauw S L, Connell P P, Weichselbaum R R. New paradigms and future challenges in radiation oncology: an update of biological targets and technology. Sci Transl Med. 2013 Feb. 20; 5(173): 173sr2.
Limagne E, Richard C, Thibaudin M, Fumet J D, Truntzer C, Lagrange A, Favier L, Coudert B, Ghiringhelli F. Tim-3/galectin-9 pathway and mMDSC control primary and secondary resistances to PD-1 blockade in lung cancer patients. Oncoimmunology. 2019 Jan. 22; 8(4): c1564505.
Liu C, Yang Z, Li R, Wu Y, Chi M, Gao S, Sun X, Meng X, Wang B. Potential roles of N6-methyladenosine (m6A) in immune cells. J Transl Med. 2021 Jun. 8; 19(1): 251.
Luo J L, Kamata H, Karin M. IKK/NF-kappaB signaling: balancing life and death—a new approach to cancer therapy. J Clin Invest. 2005 October; 115(10): 2625-32.
Mori N, Yamada Y, Ikeda S, Yamasaki Y, Tsukasaki K, Tanaka Y. Tomonaga M, Yamamoto N. Fujii M. Bay 11-7082 inhibits transcription factor NF-kappaB and induces apoptosis of HTLV-I-infected T-cell lines and primary adult T-cell leukemia cells. Blood. 2002 Sep. 1; 100(5): 1828-34.
Paris J, Morgan M, Campos J, Spencer G J, Shmakova A, Ivanova I, Mapperley C, Lawson H, Wotherspoon D A, Sepulveda C, Vukovic M. Allen L, Sarapuu A, Tavosanis A, Guitart A V. Villacreces A, Much C, Choe J, Azar A, van de Lagemaat L N, Vernimmen D, Nehme A, Mazurier F, Somervaille TCP, Gregory R I, O'Carroll D, Kranc K R. Targeting the RNA m6A Reader YTHDF2 Selectively Compromises Cancer Stem Cells in Acute Myeloid Leukemia. Cell Stem Cell. 2019 Jul. 3; 25(1): 137-148.c6.
Roundtree I A. Evans M E, Pan T. He C. Dynamic RNA Modifications in Gene Expression Regulation. Cell. 2017 Jun. 15; 169(7): 1187-1200.
Saraiva M. Christensen J R, Tsytsykova A V, Goldfeld A E, Ley S C, Kioussis D, O'Garra
A. Identification of a macrophage-specific chromatin signature in the IL-10 locus. J Immunol. 2005 Jul. 15; 175(2): 1041-6.
Shi H, Wang X, Lu Z, Zhao B S, Ma H, Hsu P J, Liu C. He C. YTHDF3 facilitates translation and decay of N6-methyladenosine-modified RNA. Cell Res. 2017 March; 27(3): 315-328.
Shields J D, Kourtis I C, Tomei A A, Roberts J M, Swartz M A. Induction of lymphoidlike stroma and immune escape by tumors that express the chemokine CCL21. Science. 2010 May 7; 328(5979): 749-52.
Si W. Liang H, Bugno J, Xu Q, Ding X. Yang K, Fu Y, Weichselbaum R R, Zhao X, Wang. L. Lactobacillus rhamnosus G G induces cGAS/STING-dependent type I interferon and improves response to immune checkpoint blockade. Gut. 2021 Mar. 8: gutjnl-2020-323426.
Sun H, Gong S, Carmody R J, Hilliard A, Li L, Sun J, Kong L, Xu L, Hilliard B, Hu S. Shen H, Yang X, Chen Y H. TIPE2, a negative regulator of innate and adaptive immunity that maintains immune homeostasis. Cell. 2008 May 2; 133(3): 415-26.
Takenaka M C, Araujo L P, Maricato J T, Nascimento V M, Guereschi M G, Rezende R M, Quintana F J, Basso A S. Norepinephrine Controls Effector T Cell Differentiation through B2-Adrenergic Receptor-Mediated Inhibition of NF-κB and AP-1 in Dendritic Cells. J Immunol. 2016 Jan. 15; 196(2): 637-44.
Taniguchi K, Karin M. NF-κB, inflammation, immunity and cancer: coming of age. Nat Rev Immunol. 2018 May; 18(5): 309-324.
Theelen W S M E, Peulen H M U, Lalezari F, van der Noort V, de Vries J F, Aerts J G J V, Dumoulin D W, Bahce I, Niemeijer A N, de Langen A J, Monkhorst K, Baas P. Effect of Pembrolizumab After Stereotactic Body Radiotherapy vs Pembrolizumab Alone on Tumor Response in Patients With Advanced Non-Small Cell Lung Cancer: Results of the PEMBRO-R T Phase 2 Randomized Clinical Trial. JAMA Oncol. 2019 Sep. 1; 5(9): 1276-1282.
Turchan W T, Pitroda S P, Weichselbaum R R. Radiotherapy and Immunotherapy Combinations in the Treatment of Patients with Metastatic Disease: Current Status and Future Focus. Clin Cancer Res. 2021 Jun. 17.
Wang X, He C. Dynamic RNA modifications in posttranscriptional regulation. Mol Cell. 2014 Oct. 2; 56(1): 5-12.
Wang X, Zhao B S, Roundtree I A, Lu Z, Han D, Ma H, Weng X, Chen K, Shi H, He C. N(6)-methyladenosine Modulates Messenger RNA Translation Efficiency. Cell. 2015 Jun. 4; 161(6): 1388-99.
Wang X, Lu Z, Gomez A, Hon G C, Yue Y, Han D, Fu Y, Parisien M, Dai Q, Jia G. Ren B, Pan T, He C. N6-methyladenosine-dependent regulation of messenger RNA stability. Nature. 2014 Jan. 2; 505(7481): 117-20.
Weichselbaum R R, Liang H, Deng L, Fu Y X. Radiotherapy and immunotherapy: a beneficial liaison? Nat Rev Clin Oncol. 2017 June; 14(6): 365-379.
Xie H, Li J, Ying Y, Yan H, Jin K, Ma X, He L, Xu X, Liu B, Wang X, Zheng X, Xie L. METTL3/YTHDF2 m6 A axis promotes tumorigenesis by degrading SETD7 and KLF4 mRNAs in bladder cancer. J Cell Mol Med. 2020 April; 24(7): 4092-4104.
Zhao B S, Wang X, Beadell A V, Lu Z, Shi H, Kuuspalu A, Ho R K, He C. m6A-dependent maternal mRNA clearance facilitates zebrafish maternal-to-zygotic transition. Nature. 2017 Feb. 23; 542(7642): 475-478.
Zheng S L, Li Z Y, Song J, Liu J M, Miao C Y. Metrnl: a secreted protein with new emerging functions. Acta Pharmacol Sin. 2016 May; 37(5): 571-9.

What is claimed is:

1. A method for treating cancer in a subject, the method comprising administering to the subject a therapeutically effective amount of:
(a) a YTHDF2 inhibitor; and
(b) a cancer therapy.

2. A method of treating cancer in a subject, the method comprising inhibiting NF-κB signaling in the subject by administering to the subject a therapeutically effective amount of:
(a) a YTHDF2 inhibitor; and
(b) a cancer therapy.

3. A method of treating cancer in a subject, the method comprising decreasing myeloid-derived suppressor cell trafficking and function in the subject by administering to the subject a therapeutically effective amount of:
(a) a YTHDF2 inhibitor; and
(b) a cancer therapy.

4. A method of increasing the efficacy of a cancer therapy in a subject, the method comprising administering to the subject a therapeutically effective amount of:
(a) a YTHDF2 inhibitor; and
(b) the cancer therapy.

5. The method of any one of claims 1-4, wherein the cancer therapy comprises immunotherapy.

6. The method of claim 5, wherein the immunotherapy comprises checkpoint blockade therapy.

7. The method of any one of claims 1-6, wherein the cancer therapy comprises radiotherapy.

8. The method of any one of claims 1-7, wherein the YTHDF2 inhibitor is an oligonucleotide targeting YTHDF2 mRNA.

9. The method of claim 8, wherein the oligonucleotide is a YTHDF2-targeting siRNA, shRNA, or antisense oligonucleotide.

10. The method of claim 8 or claim 9, wherein the oligonucleotide is an inhibitor of interaction of YTHDF2 protein with m⁶A-containing mRNA.

11. The method of any one of claims 1-7, wherein the YTHDF2 inhibitor is a small molecule inhibitor of YTHDF2.

12. The method of any one of claims 1-11, wherein the YTHDF2 inhibitor and the cancer therapy are administered substantially simultaneously.

13. The method of any one of claims 1-11, wherein the YTHDF2 inhibitor and the cancer therapy are administered sequentially.

14. The method of claim 13, wherein the YTHDF2 inhibitor is administered before the cancer therapy.

15. The method of claim 13, wherein the YTHDF2 inhibitor is administered after the cancer therapy.

16. The method of any one of claims 1-15, wherein the YTHDF2 inhibitor and the cancer therapy are administered via the same route of administration.

17. The method of any one of claims 1-15, wherein the YTHDF2 inhibitor and the cancer therapy are administered via different routes of administration.

18. The method of any one of claims 1-17, further comprising administering to the subject an additional cancer therapy.

19. The method of claim 18, wherein the additional cancer therapy comprises immunotherapy.

20. The method of claim 19, wherein the immunotherapy comprises checkpoint blockade therapy.

21. The method of claim 18, wherein the additional cancer therapy comprises radiotherapy.

22. The method of any one of claims 1-21, wherein the cancer is glioma, sarcoma, liver, lung, colon, or melanoma.

23. The method of any one of claims 1-22, wherein the cancer is metastatic and/or a solid tumor.

24. The method of any one of claims 1-23, wherein the subject was previously treated for the cancer.

25. The method of claim 24, wherein the subject was determined to be resistant to the previous treatment.

26. The method of claim 24 or claim 25, wherein the previous treatment comprised radiotherapy or immunotherapy.

27. The method of any one of claims 1-26, wherein administration of the YTHDF2 inhibitor decreases suppression of an immune response to the cancer therapy.

28. The method of any one of claims 1-27, wherein administration of the YTHDF2 inhibitor inhibits NF-κB signaling in myeloid-derived suppressor cells in the subject.

29. The method of any one of claims 1-28, wherein administration of the YTHDF2 inhibitor decreases myeloid-derived suppressor cell trafficking and function in the subject.

30. The method of any one of claims 1-29, wherein the subject was determined to have a suppressed immune response to the cancer therapy.

31. The method of claim 30, wherein the suppressed immune system response is relative to a standard.

32. The method of claim 31, wherein the standard is a measured immune response from an individual that was responsive to the cancer therapy.

33. The method of any one of claims 1-29, wherein the subject was determined to be at risk of a suppressed immune response.

34. The method of claim 33, wherein the suppressed immune system response is relative to a standard.

35. The method of claim 34, wherein the standard is a measured immune response from an individual that was responsive to the cancer therapy.

36. A method of inhibiting NF-κB signaling in myeloid-derived suppressor cells in a subject, the method comprising administering to the subject a therapeutically effective amount of a YTHDF2 inhibitor.

37. A method of decreasing myeloid-derived suppressor cell trafficking and function in a subject, the method comprising administering to the subject a therapeutically effective amount of a YTHDF2 inhibitor.

38. The method of claim 36 or claim 37, wherein the YTHDF2 inhibitor is an oligonucleotide targeting YTHDF2 mRNA.

39. The method of any one of claims 36-38, wherein the oligonucleotide is a YTHDF2-targeting siRNA, shRNA, or antisense oligonucleotide.

40. The method of claim 39, wherein the oligonucleotide is an inhibitor of interaction of YTHDF2 protein with m⁶A-containing mRNA.

41. The method of any one of claims 36-37, wherein the YTHDF2 inhibitor is a small molecule inhibitor of YTHDF2.

42. The method of any one of claims 36-41, further comprising administering to the subject a cancer therapy.

43. The method of claim 42, wherein the cancer therapy comprises immunotherapy.

44. The method of claim 43, wherein the immunotherapy comprises checkpoint blockade therapy.

45. The method of any one of claims 42-44, wherein the cancer therapy comprises radiotherapy.

46. The method of any one of claims 42-45, wherein the cancer therapy comprises an therapeutically effective amount of the cancer therapy.

47. The method of any one of claims 36-45, wherein administration of the YTHDF2 inhibitor decreases suppression of an immune response to the cancer therapy.

48. A pharmaceutical composition comprising:
(a) a YTHDF2 inhibitor; and
(b) a cancer therapeutic.

49. The pharmaceutical composition of claim 48, wherein the cancer therapeutic is an immunotherapeutic.

50. The pharmaceutical composition of claim 49, wherein the immunotherapeutic is an immune checkpoint inhibitor.

51. The pharmaceutical composition of any one of claims 48-50, wherein the YTHDF2 inhibitor is an oligonucleotide targeting YTHDF2 mRNA.

52. The pharmaceutical composition of claim 51, wherein the oligonucleotide is a YTHDF2-targeting siRNA, shRNA, or antisense oligonucleotide.

53. The pharmaceutical composition of claim 51 or claim 52, wherein the oligonucleotide is an inhibitor of interaction of YTHDF2 protein with m⁶A-containing mRNA.

54. The pharmaceutical composition of any one of claims 48-53, wherein the YTHDF2 inhibitor is a small molecule inhibitor of YTHDF2.

55. The pharmaceutical composition of any one of claims 48-54, further comprising a pharmaceutically acceptable carrier.

56. The pharmaceutical composition of any one of claims 48-55, wherein the pharmaceutical composition is formulated for injection, oral administration, intraperitoneal administration, subcutaneous administration, topical administration, intradermal administration, inhalation, intrapulmonary administration, rectal administration, vaginal administration, sublingual administration, intramuscular administration, intravenous administration, intraarterial administration, intrathecal administration, or intralymphatic administration.

57. A method of detecting YTHDF2 in a cancer patient, the method comprising measuring a level of a YTHDF2 gene product in peripheral blood mononuclear cells (PBMCs) from at least one biological sample from the cancer patient, wherein at least one of the biological samples is taken from the cancer patient:
(a) after the cancer patient has received radiotherapy; and/or
(b) during a dosing regimen of an immunotherapy to the patient and/or after the cancer patient has received the immunotherapy.

58. The method of claim 57, wherein the cancer patient has, is suspected of having, or is diagnosed with having lung cancer.

59. The method of claim 57 or 58, wherein measuring the level of the YTHDF2 gene product comprises measuring YTHDF2 protein levels.

60. The method of any one of claims 57-59, wherein measuring the level of the YTHDF2 gene product comprises measuring YTHDF2 RNA levels.

61. The method of any one of claims 57-60, wherein the PBMCs comprise myeloid-derived suppressor cells.

62. The method of any one of claims 57-61, wherein the PBMCs are enriched for myeloid-derived suppressor cells.

63. The method of any one of claims 57-62, wherein non-myeloid-derived suppressor cells are removed from the PBMCs.

64. The method of any one of claims 57-63, wherein the immunotherapy comprises a checkpoint blockade therapy.

65. The method of claim 64, wherein the checkpoint blockade therapy comprises pembrolizumab.

66. The method of claim 64 or 65, wherein the checkpoint blockade therapy comprises ipilimumab.

67. The method of any one of claims 57-66, further comprising comparing the level of the YTHDF2 gene product to a standard.

68. The method of claim 67, wherein the standard is a level of YTHDF2 present in PBMCs taken from a patient that responded to radiotherapy and/or immunotherapy.

69. The method of any one of claims 57-68, further comprising providing an additional therapy to the cancer patient based on the level of the YTHDF2 gene product.

70. The method of claim 69, wherein the additional therapy comprises a YTHDF2 inhibitor.

71. The method of claim 69 or 70, wherein the additional therapy comprises a radiotherapy and/or immunotherapy.

72. The method of claim 71, wherein the immunotherapy comprises a checkpoint blockade therapy.

73. The method of claim 72, wherein the checkpoint blockade therapy comprises pembrolizumab and/or ipilimumab.

74. A method of determining a likelihood of a cancer patient's responsiveness to a radiotherapy and/or immunotherapy, the method comprising measuring a level of a YTHDF2 gene product in peripheral blood mononuclear cells (PBMCs) taken from at least one biological sample from the cancer patient.

75. The method of claim 74, wherein the measuring occurs before an administration of the radiotherapy and/or immunotherapy.

76. The method of claim 74 or 75, wherein the measuring occurs after an administration of the radiotherapy and/or immunotherapy.

77. The method of any one of claims 74-76, wherein the cancer patient has, is suspected of having, or is diagnosed with having lung cancer.

78. The method of any one of claims 74-77, wherein the PBMCs comprise myeloid-derived suppressor cells.

79. The method of any one of claims 74-78, wherein the PBMCs are enriched for myeloid-derived suppressor cells.

80. The method of any one of claims 74-79, wherein non-myeloid-derived suppressor cells are removed from the PBMCs.

81. The method of any one of claims 74-80, wherein the immunotherapy comprises a checkpoint blockade therapy.

82. The method of claim 81, wherein the checkpoint blockade therapy comprises pembrolizumab.

83. The method of claim 81 or 82, wherein the checkpoint blockade therapy comprises ipilimumab.

84. The method of any one of claims 74-83, wherein measuring the level of the YTHDF2 gene product comprises measuring YTHDF2 protein levels.

85. The method of any one of claims 74-84, wherein measuring the level of the YTHDF2 gene product comprises measuring YTHDF2 RNA levels.

86. The method of any one of claims 74-85, further comprising comparing the level of the YTHDF2 gene product to a standard.

87. The method of claim 86, wherein the standard is a level of a YTHDF2 gene product present in PBMCs taken from a patient that responded to radiotherapy and/or immunotherapy.

88. The method of any one of claims 74-87, further comprising providing an additional therapy to the cancer patient based on the level of the YTHDF2 gene product.

89. The method of claim 88, wherein the additional therapy comprises a YTHDF2 inhibitor.

90. The method of claim 88 or 89, wherein the additional therapy comprises a radiotherapy and/or immunotherapy.

91. The method of claim 90, wherein the immunotherapy comprises a checkpoint blockade therapy.

92. The method of claim 91, wherein the checkpoint blockade therapy comprises pembrolizumab and/or ipilimumab.
